(19) United States
(12) Patent Application Publication
(10) Pub. No.: US 2025/0066842 A1
(43) Pub. Date: Feb. 27, 2025

(54) SPLIT-LUCIFERASE REPORTER SYSTEMS, COLOR-CODED BEAD MULTIPLEX CRISPR SYSTEMS, AND METHODS OF USE THEREOF IN CRISPR-CAS BASED DIAGNOSTICS

(71) Applicants: MASSACHUSETTS INSTITUTE OF TECHNOLOGY, Cambridge, MA (US); PRESIDENT AND FELLOWS OF HARVARD COLLEGE, Cambridge, MA (US); THE TRUSTEES OF PRINCETON UNIVERSITY, Princeton, NJ (US)

(72) Inventors: Pardis Sabeti, Cambridge, MA (US); Cameron Myhrvold, Princeton, NJ (US); Sameed Siddiqui, Cambridge, MA (US)

(21) Appl. No.: 18/814,007

(22) Filed: Aug. 23, 2024

Related U.S. Application Data
(60) Provisional application No. 63/534,741, filed on Aug. 25, 2023.

(51) Int. Cl. C12Q 1/682; C12N 9/02; C12N 9/22; C12N 15/11

(52) U.S. Cl. CPC C12Q 1/682; C12N 9/0069; C12N 9/22; C12N 15/11; C12Y 113/12013; C12N 2310/20; C12N 2320/10

(57) ABSTRACT

Cas-cleavable reporter systems, CRISPR-Cas systems thereof, and methods of use thereof in CRISPR-Cas based diagnostics. Cas-cleavable reporter systems may generate a luminescent, fluorescent, or other detectable signal upon Cas-collateral cleavage of one or more reporter system components. CRISPR-Cas systems may comprise a Cas protein having collateral cleavage activity, guide molecules, and the Cas-cleavable reporter system. Cas-cleavable reporter systems may be a split luciferase reporter system, at least one element of which is bound to beads. For multiplexing, CRISPR-Cas systems may comprise a Cas-cleavable quenched reporter system, optionally a Cas-cleavable quenched fluorescent reporter system, a Cas protein having collateral cleavage activity, and target-specific guide molecules attached to color-coded beads. CRISPR-Cas systems may further comprise amplification reagents. Methods may apply said CRISPR-Cas systems to flow cell, well-plate, or other devices, and may measure detectable signals by microscopic, plate reader, or other methods.

### CROSS-REFERENCE TO RELATED APPLICATIONS

This application claims the benefit of U.S. Provisional Application No. 63/534,741, filed on Aug. 25, 2023. The entire contents of the above-identified application are hereby fully incorporated herein by reference.

### STATEMENT REGARDING FEDERALLY SPONSORED RESEARCH

This invention was made with government support under Grant No. AC00006 awarded by the Defense Advanced Research Projects Agency (DARPA), and Grant No. 75D30122C15113 awarded by the Centers for Disease Control. The government has certain rights in the invention.

### REFERENCE TO AN ELECTRONIC SEQUENCE LISTING

The contents of the electronic sequence listing (“BROD-5915US_ST26.xml; Size is 97,505 bytes and it was created on Aug. 23, 2024) is herein incorporated by reference in its entirety.

### TECHNICAL FIELD

The subject matter disclosed herein is generally directed to split-luciferase reporter systems, color-coded bead multiplex CRISPR systems, and methods of use thereof in CRISPR-Cas based diagnostics.

### BACKGROUND

The COVID-19 pandemic has highlighted the need for rapidly deployable diagnostic technologies able to respond to new pathogens and emergent variants anywhere in the world (Liu et al. 2021). However, no current technology uniquely meets the sensitivity, specificity, deployability, speed, and multiplexing needs required for a broad and robust response to infectious disease outbreaks. Quantitative polymerase chain replication (qPCR), widely considered a gold standard due to its sensitivity and specificity, cannot be deployed readily, and remains limited in multiplexing ability (Eckbo et al. 2021; Center for Devices and Radiological Health n.d.; Knox and Beddoe 2021). Next generation sequencing (NGS) is similarly sensitive, specific, and able to detect many pathogens and variants; however, it is expensive, has a long turnaround time, and requires significant technical expertise to deploy for viral surveillance (Goodwin, McPherson, and McCombie 2016). On the other hand, antigen capture tests are readily deployable and affordable, but are less sensitive and specific than nucleic acid tests and are not rapidly adaptable for new pathogens (Arizti-Sanz et al. 2020; Chu et al. 2022).

CRISPR-Cas based systems offer an alternative approach that is well-poised to address pathogen diagnostic needs. Specifically, CRISPR effectors Cas12 and Cas13 exhibit collateral cleavage activity upon recognition of their target DNA or RNA, respectively, enabling these enzymes to act as target-specific sensors (Chen et al. 2018; Liu et al. 2021; Gootenberg et al. 2017; East-Seletsky et al. 2016). Since their introduction, there has been substantial work in developing CCRISPR-Cas based diagnostic systems, with assays for diverse pathogens such as influenza, Zika virus, and SARS-CoV-2 developed across different combinations of CRISPR effectors, imaging tools, and reaction platforms (Pardee et al. 2016; Liu et al. 2021; Park et al. 2021; Fozouni et al. 2021). Much of this effort has been limited to fluorescence and lateral flow strip readouts, leaving room for orthogonal advances in reporter design and reaction barcoding to improve deployability, sensitivity, and multiplexing capability (Myhrvold et al. 2018; Barnes et al. 2020; Welch et al. 2022; Arizti-Sanz et al. 2020; Ackerman et al. 2020; Arizti-Sanz et al. 2022; Liu et al. 2021).

Citation or identification of any document in this application is not an admission that such a document is available as prior art to the present invention.

### SUMMARY

In an aspect, the present invention provides a split luciferase reporter system. In an example embodiment, the split luciferase reporter system comprises: a first reporter protein subunit coupled to first bead or a masking agent via a first oligonucleotide linker capable of being cleaved by Cas nuclease activity, optionally Cas collateral nuclease activity; a second reporter protein subunit optionally coupled to a second bead via a second linker, optionally a second oligonucleotide linker, wherein the first and second reporter protein subunits can complex to generate a detectable luminescent signal, and wherein the masking agent or the bead prevents generation of the detectable luminescent signal. In an example embodiment, the first oligonucleotide linker and/or the second linker is selected from the group consisting of a DNA sequence, an RNA sequence, or any combination thereof. In an example embodiment, the first oligonucleotide linker and/or the second linker comprises at least 5, at least 6, at least 7, at least 8, at least 9, or at least 10 nucleotides. In an example embodiment, the first oligonucleotide linker and/or the second linker is selected from an oligonucleotide of Table 1 or a derivative thereof. In an example embodiment, the split luciferase reporter system further comprises a reacting reagent. In an example embodiment, the reacting reagent is furimazine, an analogue thereof, or any combination thereof. In an example embodiment, the first protein subunit is HiBiT and the second protein subunit is LgBiT.

In an example embodiment, the first bead and/or the second bead are selected from the group consisting of bovine serum albumin (BSA) coated beads, streptavidin coated beads, amine-functionalized beads, thiol-functionalized beads, agarose beads, polystyrene beads, magnetic beads, hydrophilic beads, and any combination thereof, and wherein, optionally, the beads are nanobeads or microbeads. In an example embodiment, the first oligonucleotide linker and/or the second linker connects to a corresponding protein subunit, a corresponding bead, and/or the masking construct, using: a click chemistry connection, an amine coupling connection, a thiol coupling connection, or any combination thereof, a Strain-Promoted Azide-Alkyne Click Chemistry (SPAAC) reaction connection, a maleimide-thiol reaction connection, an amine-N-hydroxysuccinamide reaction connection, or any combination thereof, a biotin-streptavidin connection; an antibody-coupling connection; and/or a HaloTag-HaloLigand connection.

In an example embodiment, the masking agent is a luminescence inactivator, wherein the first protein subunit and the luminescence inactivator are in a catalytically inactive complex which does not generate a luminescent signal upon exposure to a reacting agent, and wherein the catalytically inactive complex is disrupted upon Cas-cleavage of oligonucleotide linker. In an example embodiment, the first protein subunit is LgBiT and the second protein subunit is HiBiT. In an example embodiment, the luminescence inactivator is a catalytically inactive third protein or peptide subunit, optionally DrkBiT. In an example embodiment, the first oligonucleotide linker covalently connects the first protein subunit and the luminescence inactivator; or wherein the luminescence inactivator is connected to a 5′ oligonucleotide and the first protein subunit is connected to a 3′ oligonucleotide or vice versa, wherein the 5′ and the 3′ oligonucleotides are capable of hybridizing with the Cas-cleavable oligonucleotide linker at positions 5′ and 3′, respectively, of the Cas-cleavable oligonucleotide sequence, such that first protein or peptide subunit and the luminescence inactivator form a catalytically inactive bound complex upon hybridization to the Cas-cleavable oligonucleotide linker. In an example embodiment, the luminescence inactivator is connected to the 5′ or the 3′ oligonucleotide using a click chemistry connection; and/or wherein the first protein subunit is connected to the 5′ or the 3′ oligonucleotide using a HaloLigand connection. In an example embodiment, the split luciferase reporter system further comprises a luminescence inactivator that is not connected to the first protein subunit.

In an aspect, the present invention provides a detection system for detecting one or more target oligonucleotides in a sample. In an example embodiment, the method comprises: a split luciferase reporter system of the present invention; and a CRISPR-Cas system comprising; a Cas having collateral activity; and one or more guide molecules capable of forming a complex with the Cas and directing sequence-specific binding to one or more corresponding target oligonucleotide sequences, wherein, upon activation of the Cas, the Cas collateral activity cleaves first and optionally the second oligonucleotide linker, thereby generating a detectable luminescent signal indicating a presence of one or more of the target oligonucleotide sequences in the sample. In an example embodiment, the Cas has collateral DNA cleavage activity, collateral RNA cleavage activity, or any combination thereof. In an example embodiment, the Cas having collateral cleavage activity is a Type V Cas, a Type VI Cas, or any combination thereof.

In an example embodiment, the detection system further comprises nucleic acid amplification reagents. In an example embodiment, the nucleic acid amplification reagents comprise nucleic acid sequence-based amplification (NASBA) reagents, recombinase polymerase amplification (RPA) reagents, loop-mediated isothermal amplification (LAMP) reagents, strand displacement amplification (SDA) reagents, helicase-dependent amplification (HDA) reagents, nicking enzyme amplification reaction (NEAR) reagents, PCR reagents, multiple displacement amplification (MDA) reagents, rolling circle amplification (RCA) reagents, ligase chain reaction (LCR) reagents, or ramification amplification method (RAM) reagents.

In an aspect, the present invention provides a detection method for detecting one or more target oligonucleotides in a sample. In an example embodiment, the detection method comprises: contacting the sample with the split-luciferase reporter CRISPR-Cas detection system of the present invention, wherein the generation of the detectable luminescent signal indicates the presence of one or more of the target oligonucleotide sequences in the sample. In an example embodiment, the detection method further comprises detecting the detectable luminescent signal. In an example embodiment, the detecting occurs for a period of time from 25 minutes to 3 hours. In an example embodiment, the sample is applied to a lateral flow immunochromatographic assay prior to or after the contacting occurs.

In an aspect, the present invention provides a color-coded bead multiplex CRISPR-Cas detection system for detecting one or more target oligonucleotides in a sample. In an example embodiment, the system comprises; a Cas having collateral cleavage activity; a set of guide molecules capable of forming a complex with the Cas and directing sequencing specific binding to a set of target oligonucleotide sequences, wherein each guide molecule is bound to a color-coded bead specifically identifying said guide molecule, and thereby the target oligonucleotide sequence specific for said guide molecule, and one or more oligonucleotide-based detection constructs comprising a non-target sequence susceptible to the Cas collateral cleavage activity, wherein, upon activation of the Cas by the binding of CRISPR-Cas complex to one or more of the target sequences, the Cas collateral activity cleaves the non-target sequence, thereby generating a detectable signal indicating a presence of one or more of the target oligonucleotide sequences in the sample.

In an example embodiment, the color-coded beads are selected from the group consisting of bovine serum albumin (BSA) coated beads, streptavidin coated beads, agarose beads, polystyrene beads, magnetic beads, hydrophilic beads, and any combination thereof, and wherein, optionally, the beads are nanobeads or microbeads. In an example embodiment, the guide molecules are bound to the beads by a biotin-streptavidin connection. In an example embodiment, the oligonucleotide-based detection constructs comprise an oligonucleotide to which a detectable ligand and a masking component are attached. In an example embodiment, the detectable ligand is a fluorophore and the masking component is a quencher molecule.

In an example embodiment, the non-target sequence is selected from the group consisting of a DNA sequence, an RNA sequence, or any combination thereof. In an example embodiment, the Cas has collateral DNA cleavage activity, collateral RNA cleavage activity, or any combination thereof. In an example embodiment, the Cas having collateral cleavage activity is a Cas12, a Cas13, or any combination thereof.

In an example embodiment, the detection CRISPR system further comprises nucleic acid amplification reagents. In an example embodiment, the nucleic acid amplification reagents comprise nucleic acid sequence-based amplification (NASBA) reagents, recombinase polymerase amplification (RPA) reagents, loop-mediated isothermal amplification (LAMP) reagents, strand displacement amplification (SDA) reagents, helicase-dependent amplification (HDA) reagents, nicking enzyme amplification reaction (NEAR) reagents, PCR reagents, multiple displacement amplification (MDA) reagents, rolling circle amplification (RCA) reagents, ligase chain reaction (LCR) reagents, or ramification amplification method (RAM) reagents.

In an example embodiment, the color-coded bead multiplex CRISPR-Cas detection system further comprises a pool of droplets comprising a sample and the color-coded bead multiplex CRISPR-Cas detection system, wherein each droplet comprises a single one of the color-coded beads, such that, detection of the detectable signal and identifying the color of the color-coded bead within one of the droplets indicates a presence in the sample of the target oligonucleotide sequence corresponding to the guide molecule identified by the color-coded bead in said droplet. In an example embodiment, the pool of droplets is an oil-in-water emulsion, wherein, optionally, the oil further comprises a surfactant. In an example embodiment, the oil is a fluorous oil, and wherein the surfactant is a fluorosurfactant.

In an aspect, the present invention provides a color-coded bead multiplex CRISPR-Cas detection method for detecting a target sequence in a sample. In an example embodiment, the color-coded bead CRISPR-Cas detection method comprises: contacting a sample with a color-coded bead multiplex CRISPR-Cas detection system of the present invention, thereby forming a pre-mixture; mixing the pre-mixture with a solvent immiscible with the pre-mixture, thereby forming a pool of droplets comprising the sample and the detection CRISPR system; detecting each droplet generating a detectable signal and comprising a single one of the color-coded beads; and identifying the color-code of the single bead within said droplet, thereby indicating a presence in the sample of the target sequence corresponding to the guide molecule identified by the single color-coded bead in said droplet. In an example embodiment, the pool of droplets is an oil-in-water emulsion, wherein, optionally, the oil further comprises a surfactant. In an example embodiment, the oil is a fluorous oil, and wherein the surfactant is a fluorinated surfactant. In an example embodiment, prior to the detecting steps, the method further comprises loading the pool of droplets onto a flow cell, and wherein the detecting steps are performed with a microscope; or prior to the detecting steps, the method further comprises loading the pool of droplets onto a well plate, and wherein the detecting steps are performed with a plate reader.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Bead-based strategy to couple Cas-13 activity with a split NanoLuciferase-based readout. (FIG. 1A) Schematic of bead-based Luciferase reporter (bbLuc) assay. (FIG. 1B) Attachment of HiBiT and LgBiT to beads using Cas-cleavable RNA-based linker. (FIG. 1C) Comparison of Cas13-based and RNase A-based cleavage of HiBiT-linked beads. (FIG. 1D). Limit of detection of bbLuc assay. (FIG. 1E) bbLuc assay sensitivity as a function of HiBiT peptide bead surface density. (FIG. 1F) Comparison of bbLuc assay and Cas-13-based assay using fluorescent reporter.

FIG. 2—(FIG. 2A) Comparison of amplification-free bbLuc assay vs. bbLuc-SHINE assay vs. Cas13-based assay with fluorescent reporter vs. SHINE (Streamlined Highlighting of Infections to Navigate Epidemics) assay with fluorescent reporter (FIG. 2B) bbLuc Linker design variations to reduce detection inhibition by recombinase polymerization amplification (RPA) components. (FIG. 2C) Effect of alternative bbLuc linker designs on bbLuc assay sensitivity. (FIG. 2D) Limit of detection (LOD) of optimized bbLuc assay. (FIGS. 2E-2F) Comparison of bbLuc and fluorescent SHINE assays to a gold-standard RT-qPCR on RNA extracted from 63 clinical swabs from suspected COVID-19 patients.

FIG. 3—Equipment-free bead-based droplet generation for multiplexed fluorescent Cas13 detection. (FIG. 3A) Schematic of bead-based CARMEN (Combinatorial Arrayed Reactions for Multiplexed Evaluation of Nucleic acids) (bbCARMEN) assay using biotinylated crRNAs attached to color-coded, streptavidin-coated beads. (FIGS. 3B-3E) Flow cell assay for bbCARMEN assay SARS-Cov-2 and RNase P in patient samples using fluorescence microscopy and automated image analysis to track both the color-coded crRNA beads and the signal from Cas13 activity.

FIG. 4—(FIG. 4A) Evaluation of bbCARMEN for multiplexed detection of 9-target Respiratory Virus Panel (RVP). (FIGS. 4B-4C) Determination of LOD and off-target detection of bbCARMEN. (FIG. 4D) Validation of bbCARMEN for detection of SARS-CoV-2. (FIG. 4E) Comparison of bbCARMEN vs multiplexed CARMEN (mCARMEN) assays for SARS-CoV-2.

FIG. 5—Automated readout of bbCARMEN. (FIG. 5A) bbCARMEN adapted for a standard well plate and a lab plate reader. (FIGS. 5B-5D) Concordance of assay results between microscopy-based and plate reader-based assays.

FIG. 6—(FIG. 6A) Fluorescence vs (FIG. 6B) Split Luciferase Luminescent System. (FIG. 6C) Luminescence is up to 1000× more sensitive than fluorescence.

FIG. 7—Optimization of LgBiT Concentration (Bead Surface Density).

FIG. 8—Effect of Polyethylene Glycol (PEG) Buffers.

FIG. 9—Interference in Detection by RPA pellet components.

FIG. 10—Interference in Detection by Single-Stranded Binding (SSB) Protein and Recombinases in RPA.

FIG. 11—Comparison of Alternative bbLuc Linkers.

FIG. 12—Identification of Top Performing Alternative bbLuc Linkers.

FIG. 13—Effect of Modifying Furimazine Concentration.

FIG. 14—Effect of Modifying RPA Primer Concentration.

FIG. 15—Effect of Modifying Magnesium Concentration.

FIG. 16—Comparison of CARMEN with and without beads. (FIG. 16A) Schematic bbCARMEN vs mCARMEN assay. (FIGS. 16B-16D) Flow cell assay for bbCARMEN and CARMEN assays using fluorescence microscopy and automated image analysis to track both the color-coded crRNA beads and the signal from Cas13 activity.

FIG. 17—Evaluation of bbCARMEN using 5′ end or 3′ end biotinylated crRNAs. Background signal reduction with 3′ biotinylated crRNA-bead pools compared to 5′ pools. (FIG. 17A) Fluorescence across amplified SARSCoV-2 dilution series from 104-100 copies/uL at 0, 30, and 60 min post-reaction initiation. Top: 5′ biotinylated crRNA-bead pools; Bottom: 3′ biotinylated crRNA-bead pools; Green: SARS-CoV-2; Gray: NTC. Bar at median fluorescence. (FIG. 17B) Fluorescence kinetics of SARS-CoV-2 at 100 copies/uL from 3′ and 5′ biotinylated crRNA-bead pools. Green: SARS-CoV-2; Black: NTC; Closed points: 3′ biotinylated crRNA; Open points: 5′ biotinylated crRNA. (FIG. 17C) Heatmap of median SARS-CoV-2 and NTC fluorescence at 60 min post-reaction initiation from a. Asterisk (*) represents positive signal detected above threshold.

FIG. 18—Comparison of alternative fluorescent reporters for bbCARMEN. Dual SARS-CoV-2 and RNase P fluorescence imaging over time (FIG. 18A) Schematic of bbCARMEN flow cell imaging setup, showing up to 60 minutes post-reaction initiation. (FIG. 18B) Fluorescent images at 0, 30, and 60 min post-reaction in 4 different fluorescent channels. Synthetic SARS-CoV-2 RNA at 106 copies/uL was spiked into RNase P. Blue: AF647; Red: AF594; Yellow: AF564; Green: FAM. AF647: Semrock LF635-B; AF594: Semrock 3FF03-575/25-25 and FF01-615/24-25; AF546: Semrock SpGold-B; FAM: Semrock GFP-1828A. Scale bar: 500 um.

FIG. 19—Flow cell assay using bbCARMEN for multiplexed detection of 9-target Respiratory Virus Panel (RVP). bbCARMEN setup and image analysis for bead classification with RVP. (FIG. 19A) schematic of bbCARMEN reaction setup and image analysis pipeline that: 1) identifies beads within droplets by drawing a circle around a single bead in droplet, 2) draws an additional circle surrounding the identified bead for FAM signal analysis, and 3) bead color-codes are characterized and classified. (FIG. 19B) image of bead identification. (FIG. 19C) ternary plot characterizing the 12 color codes that make up the RVP bead pool.

0041 FIG. 20—Flow cell assay using bbCARMEN for multiplexed detection of 9-target Respiratory Virus Panel (RVP). Characterization of SARS-CoV-2 FAM fluorescence signal across all bead and lane replicates. (FIG. 20A) Schematic of fluorescence values derived from signals within the donuts formed around a bead within a droplet. (FIG. 20B) FAM fluorescence within donuts of each color-coded crRNA bead population that make up RVP at 0, 30, and 60 min post-reaction initiation. Bar at median fluorescence. Thresholds shown as dashed lines at each time point calculated as 3× the standard deviation of the NTC. c-e, Tukey box and whiskers plot of fluorescence values across lane replicates at 0, 30, and 60 min post-reaction_initiation. Outliers represented as single points. (FIG. 20C) NTC fluorescence. (FIG. 20D) SARS-CoV-2 fluorescence. (FIG. 20E) RNase P fluorescence.

FIG. 21—Determining LOD for 9-target Respiratory Virus Panel (RVP). RVP limit of detection evaluation on bbCARMEN. (FIG. 21A) Schematic of LOD testing with bbCARMEN. (FIG. 21B) Fluorescence of SARS-CoV-2, 2.5 copies/uL, and NTC for each of the 20 technical replicates. Individual points represent signal from a single droplet with a bar at median fluorescence. Green: SARS-CoV-2; Black: NTC. (FIG. 21C) Median fluorescence (n=20) at the LOD for each of the 9 viruses on RVP as established by spiking synthetic RNA into negative control RNA. Green: virus; Gray: RNase P; Dashed line: Threshold derived from NTC. (FIG. 21D) Comparison of RVP LODs across the CARMEN technologies. Green: bbCARMEN RVP assay; Teal: mCARMEN used w/bbCARMEN RVP crRNA; Blue: mCARMEN used w/mCARMEN RVP crRNA.

FIG. 22—Comparison of bbCARMEN to Comparator Assays. (FIG. 22C) bbCARMEN determination of percent positive results for SARS-CoV-2 over time. Comparison of bbCARMEN detection results to results collected from comparator assays. (FIG. 22A) Schematic of patient sample testing, with 79 patient samples tested, including 45 SARS-CoV-2 samples (30 positive, 15 negative) and 34 HRSV samples (30 positive, 4 negative). (FIG. 22B) Concordance of bbCARMEN and bbCARMEN for 79 patient samples. (FIG. 22C) Scatter plot of scaled normalized fluorescent values compared to viral Ct values detected by RT-qPCR at 0 min, 30 min, and 60 min timepoints for the positive SARS-CoV-2 samples.

FIG. 23—Concordance of assay results between microscopy-based and plate reader-based assays. Comparison of bbCARMEN fluorescent results from two readout platforms. (FIG. 23A) Heatmaps at 60 min post-reaction initiation. (FIG. 23B) Tukey box and whisker plots at 1e3 copies/uL 60 min post-reaction initiation; dashed line: 3 std dev above the median NTC. (FIG. 23C) Kinetic curves of runs with 1e4 and 1e3 copies of SARS-CoV-2 on the microscope and plate reader-based platforms, including scrambled crRNA NTC values for each input sample.

FIG. 24—Split-luciferase systems from NanoLuc® Binary Technology (Promega).

FIG. 25—Version 0 (V0) HiBiT beads—Coupling to streptavidin beads via HaloLigand-HaloTag.

FIG. 26—(FIG. 26A) V0 HiBiT bead—Luminescence activity. (FIG. 26B) V0 HiBiT beads cleaved by Cas13 or RNase A.

FIG. 27—V0 HiBiT beads—Effect of changing HiBiT linker length on Cas13 cleavage.

FIG. 28—V0 LgBiT beads—(FIG. 28A) Dynabeads with Protein G. (FIG. 28B) One-bead assay using LgBiT.

FIG. 29—Full assay of V0 HiBiT beads and LgBiT beads.

FIG. 30—Optimization of assay of V0 HiBiT beads and LgBiT beads.

FIG. 31—V1 HiBiT beads: Effect of click-chemistry coupling of HiBiT peptide to linker on Cas13 cleavage.

FIG. 32—Comparison of bbLuc assay and Cas-13-based assay using fluorescent reporter.

FIG. 33—Comparison of bbLuc assay and Cas-13-based assay using fluorescent reporter.

FIG. 34—Version Zero (V0) Luminescent Molecular Beacon Assay.

FIG. 35—(FIGS. 35A-35B) Effect of pre-incubation of Luminescent Molecular Beacon with Free DrkBiT.

FIG. 36—Version One (V1) Luminescent Molecular Beacons: RNase A-based cleavage is greater than background NTC.

FIG. 37—(FIGS. 37A-37B) V1 Luminescent Molecular Beacons: RNase A-based cleavage is greater than background NTC—Continued.

FIG. 38—(FIGS. 38A-38B) V1 Luminescent Molecular Beacons: Cas13-based cleavage is greater than background NTC.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment”, “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide Cas-cleavable reporter systems and methods of use thereof in CRISPR-Cas based diagnostics. In an aspect, the reporter systems are split-luciferase reporter systems or bead-based multiplex CRISPR-Cas detection systems. Embodiments disclosed herein further provide kits comprising components of said systems and devices for performing said methods.

CRISPR-Cas based diagnostics have emerged as a promising tool for fast, accurate, and portable pathogen detection. There has been rapid progress in areas such as pre-amplification processes and CRISPR-related enzymes, but the development of reporter systems and reaction platforms has lagged behind. Applicant has developed new bead-based techniques that can help fill both gaps. First, Applicant has developed a novel bead-based split-luciferase reporter system with improved sensitivity compared to standard fluorescence-based reporter design in CRISPR diagnostics. Second, Applicant has developed a highly deployable, bead-based platform capable of detecting nine distinct viral targets in parallelized, droplet-based reactions. Applicant has demonstrated the enhanced performance of both approaches on synthetic and clinical samples. Together, these systems represent new modalities in CRISPR diagnostics with increased sensitivity, speed, multiplexing, and deployability.

New bead-based systems have led to recent advances in protein detection due to their ability to compartmentalize reaction components and may serve as a basis to advance CRISPR-Cas based nucleic acid detection. For example, AlphaLISA implements a two-bead chemiluminescent reporter system that enables highly-sensitive wash-free antigen detection (Ullman et al. 1994; Bielefeld-Sevigny 2009). The two beads in the system have different surface chemistries and are coupled with complementary antibodies of a sandwich ELISA; when an antigen is bound, the complementary beads come together and emit light as a reporter for antigen detection. This suggests the possibility of a highly sensitive split luciferase reporter for Cas13 diagnostics which separates split luciferase components onto different beads prior to Cas13 target detection.

Applicant first considered how bead-based readouts could improve sensitivity in point-of-need CRISPR-Cas based diagnostic assays, such as in Streamlined Highlighting of Infections to Navigate Epidemics (SHINE), a point-of-need CRISPR diagnostic platform which increases sensitivity by coupling isothermal amplification with Cas13 detection (Arizti-Sanz et al. 2022, 2020). These assays have traditionally used fluorescence-based reporters, primarily consisting of a fluorescein (FAM) dye linked by a short oligonucleotide sequence to a quencher (Arizti-Sanz et al. 2020; Chen et al. 2018; Liu et al. 2021; Myhrvold et al. 2018). While these assays have performed well, fluorescence-based technologies are known to have high background signal and low sensitivity compared to bioluminescence technologies (Arizti-Sanz et al. 2020; Tung et al. 2016).

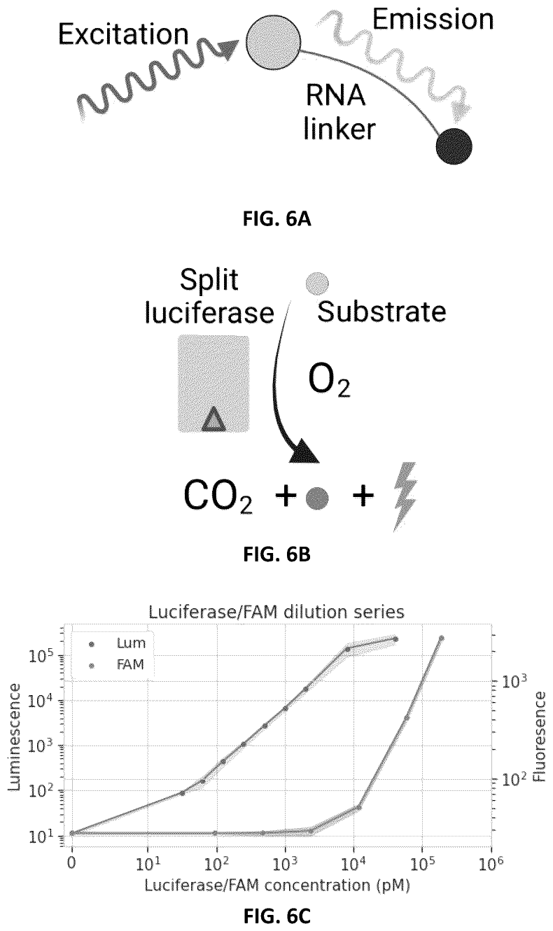

A bead-based luminescent split reporter system which links nucleic acid detection with NanoLuciferase (NanoLuc) complementation could provide an attractive alternative to fluorescent reporters, enabling rapid attomolar detection with a high dynamic range (Dixon et al. 2016; Schwinn et al. 2018; Fan and Wood 2007) (FIG. 24). A two-bead system with a large protein subunit (LgBiT) and a smaller peptide subunit (HiBiT) each coupled to separate bead type may serve as a basis for a Cas13 cleavage reporter if at least one of the protein subunits is coupled via a Cas13-cleavable RNA linker. By virtue of being coupled to separate beads, LgBiT and HiBiT can be largely separated from each other and kept catalytically inactive. In the presence of the target, Cas13 collateral cleavage of the bead RNA linkers could reverse this separation and allow the formation of complemented NanoLuc. Therefore, for point-of-need use, a new split-luciferase-based reporter system could be well-poised to improve sensitivity in amplification-free conditions while simultaneously removing the requirement of a light source as in fluorescence-based systems (FIG. 6).

Applicant next considered how a bead-based system could improve multiplexed diagnostic testing at point of care. Applicant previously developed the CRISPR-Cas based Combinatorial Arrayed Reactions for Multiplexed Evaluation of Nucleic acids (CARMEN) and microfluidic CARMEN (mCARMEN) (Welch et al. 2022; Ackerman et al. 2020) and demonstrated their unprecedented multiplexing and sensitivity across samples and pathogens. These platforms, however, require high technical expertise and costly equipment to achieve sample or patient barcoding, restricting their deployability in resource-limited settings (Ackerman et al. 2020; Welch et al. 2022). This constraint leaves an opportunity to replace barcoding with a less resource-intensive, bead-based approach.

A color-coded bead-based approach that couples beads to distinct crRNAs could be used to create a localized separation of crRNA, enabling an assay with multiplexed nucleic acid targets. Given previous work in microparticle-based dropletization, such color-coded beads could allow equipment-free nanoliter droplet generation (Clark et al. 2023), with each droplet containing Cas13 detection master mix and approximately one color-coded crRNA bead. With crRNA-specific detection reactions occurring in parallel across different droplets, this could in turn enable a highly parallelized reaction which could be imaged in a fluorescent microscope or plate reader to determine bead target-specific detection. Thus, for point-of-care use, a bead-based, low-cost platform capable of parallelized, dropletized detection of multiple targets may enable a sensitive, robust, and highly multiplexed solution for resource-limited settings.

Here, Applicant has explored the applicability of novel bead-based approaches to increasing sensitivity, multiplexing, and deployability in CRISPR-diagnostics. Applicant has designed a bead-based split luciferase reporter (bbLuc) and examined this readout modality in an amplification-free reaction and in the SHINE diagnostic platform.

### Split-Luciferase Reporter Systems

In an aspect, the present invention provides split luciferase reporter systems. In an example embodiment, a split-luciferase reporter system is sensitive to the cleavage of programmable nucleases, such as Cas nucleases. In an example embodiment, a split luciferase reporter system comprises a first reporter protein subunit coupled to a masking agent or a bead via an oligonucleotide linker, and a second reporter protein subunit, which may optionally be connected to a second bead via a linker, which may be an oligonucleotide-based linker or a non-oligonucleotide-based linker. The masking agent and/or bead(s) isolate the first and second reporter protein subunits from one another preventing generation of a detectable signal. Upon cleavage of the oligonucleotide linker, for example, by Cas collateral cleavage activity, the first and second reporter protein subunits can complex to generate a detectable signal. In one embodiment, the complexed first and second reporter protein subunits may only exhibit a detectable signal in the presence of a reacting agent. Accordingly, the system may further comprise a reacting agent as needed.

The first and second reporter protein subunits may be protein subunits of a split-luciferase reporter system. In an example embodiment, the split-luciferase reporter system comprises a large protein subunit and a small protein subunit. In an example embodiment, the large protein subunit is a LgBiT protein of a Nanoluc® Binary Technology (NanoBiT®) system obtained, for example, from Promega, and/or the small protein subunit is a SmBiT protein of a Nanoluc® Binary Technology (NanoBiT®) system obtained, for example, from PROMEGA, and/or the small protein is a HiBiT protein obtained, for example, from PROMEGA. (Dixon et al. 2016; Schwinn et al. 2018; Fan and Wood 2007). In an example embodiment, the first protein subunit is a HiBiT protein and the second protein subunit is a LgBiT protein.

### Oligonucleotide Linker

The oligonucleotide linker may comprise any oligonucleotide sequence that is cleavable by Cas nuclease activity, more preferably by Cas collateral nuclease activity. The oligonucleotide linker may be selected from the group consisting of a DNA sequence, an RNA sequence, or any combination thereof. The oligonucleotide linker may be between 5-100, 5-50, 5-45, 5-40, 5-35, 5-30, 5-25, 5-20, 5-10, 10-15, 15-20, 15-25, 25-30, 30-35, 35-40, 40-45, 45-50, 50-55, 55-60, 60-65, 65-70, 70-75, 75-80, 80-85, 85-90, or 95-100 nucleotides in length. In an example embodiment, the oligonucleotide linker is selected from an oligonucleotide of Table 1, or a derivative thereof.

### Beads

In an embodiment, the individual discrete volume is a bead. Sets of guide molecules can be comprised on a bead, and are referred to herein as a detection bead. Beads used in the present invention may comprise a hydrogel bead or a magnetic bead or a magnetic core. In an aspect of the embodiment, the bead is a polymer, for example, hydroxylated methacrylic polymer, a hydroxylated poly(methyl methacrylate), a polystyrene polymer, a polypropylene polymer, a polyethylene polymer agarose, or cellulose. The bead may have a porosity, throughout the bead, or in a portion of the bead. In another aspect of the embodiment, the bead has a shape that is circular, square, star, or generally rounded.

In particular embodiments, the bead has an average particle size or diameter between about 0.5 microns to 100 microns, about 1 micron to 100 microns, about 2 micron to 100 microns, about 3 microns to about 100 microns, about 5 micron to 90 microns, about 10 microns to about 90 microns, about 10 microns to about 80 microns, about 10 microns to about 70 microns, about 10 microns to about 60 microns, about 10 microns to about 50 microns, about 10 microns to about 40 microns, about 10 microns to 30 microns, about 10 microns to about 20 microns, about 20 microns to about 30 microns, about 20 microns to about 40 microns, about 20 microns to about 50 microns, about 20 microns to about 60 microns, about 20 microns to about 70 microns, about 20 microns to about 80 microns, about 20 microns to about 100 microns.

In a particular embodiment, the reporter bead and the detection bead are sized differently and are utilized with an individual discrete volume to hold only one of each bead type, e.g. one reporter bead and one detection bead. In an aspect, the beads can be sequentially flowed onto the microfluidic device. In an aspect, the beads are distributed to wells arrayed on a microfluidic device.

### Masking Agent

The masking agent may be a catalytically inactive third protein or peptide subunit. In an example embodiment, the luminescence inactivator is a DrkBiT protein or peptide obtained, for example, from PROMEGA. In an example embodiment, the split-luciferase reporter system further comprises a luminescence inactivator that is not connected to the first protein or peptide subunit.

### Linker Chemistry

In an example embodiment, the oligonucleotide linker connects to the corresponding protein subunit to the masking agent or bead using a click chemistry connection, an amine coupling connection, a thiol coupling connection, or any combination thereof. In an example the oligonucleotide linker connects to the first protein subunit using a Strain-Promoted Azide-Alkyne Click Chemistry (SPAAC) reaction connection, a maleimide-thiol reaction connection, an amine-N-hydroxysuccinamide reaction connection, or any combination thereof. In an example embodiment, oligonucleotide linker connects the corresponding protein subunit to the corresponding bead using a biotin-streptavidin connection. In an example embodiment, the oligonucleotide linker connects the corresponding protein subunit using an antibody-coupling connection or a HaloTag-HaloLigand connection. In an embodiment with two linkers connecting the first and second reporter protein subunits to a bead, the linkers may connect the reporter protein subunit to the bead using the same or different linker chemistry.

### Reacting Agent

In an example embodiment, the complex of the first and second reporter subunits exhibits luciferase activity in the presence of the reacting agent. In an example embodiment, the reacting agent is a substrate for luciferase enzymatic activity. In an example embodiment, a reacting agent is furimazine or an analogue thereof. (Dixon et al. 2016; Schwinn et al. 2018; Fan and Wood 2007). In an example embodiment, the split-luciferase reporter system further comprises a reacting agent.

### Split-Luciferase Reporter Crispr-Cas Detection Systems and Methods of Use Thereof

In an aspect, the present invention provides an oligonucleotide detection system comprising the split-luciferase reporters described above and further coupled with a CRISPR-Cas programmable nuclease for use in CRISPR-Cas detection systems and methods of use thereof as described herein.

CRISPR-Cas systems have been adapted for diagnostic purposes. For example, Cas12 and Cas13 exhibit collateral cleavage activity upon recognition of their target DNA or RNA, respectively, enabling these enzymes to act as target-specific sensors (Chen et al. 2018; Liu et al. 2021; Gootenberg et al. 2017; East-Seletsky et al. 2016). Since their introduction, there has been substantial work in developing CRISPR-based diagnostic systems, with assays for diverse pathogens such as influenza, Zika virus, and SARS-CoV-2 developed across different combinations of CRISPR effectors, imaging tools, and reaction platforms (Pardee et al. 2016; Liu et al. 2021; Park et al. 2021; Fozouni et al. 2021). Much of this effort has been limited to fluorescence and lateral flow strip readouts, leaving room for orthogonal advances in reporter design and reaction barcoding to improve deployability, sensitivity, and multiplexing capability (Myhrvold et al. 2018; Barnes et al. 2020; Welch et al. 2022; Arizti-Sanz et al. 2020; Ackerman et al. 2020; Arizti-Sanz et al. 2022; Liu et al. 2021).

The CRISPR-Cas system can be configured to detect one or more target oligonucleotides in a sample via selection of a spacer sequence that recognizes and binds to a corresponding target sequence in a target polynucleotide. Upon recognition of the target oligonucleotide, the Cas nuclease will then cleave the oligonucleotide of the split-luciferase systems described herein leading to generation of a detectable luminescent signal.

In an example embodiment, any CRISPR-Cas system of the present invention can be adapted for use in split-luciferase reporter CRISPR-Cas detection systems of the present invention. In an example embodiment, the CRISPR system comprises a Cas having collateral activity for the Cas-cleavable oligonucleotide linker(s) of said split-luciferase reporter system, and one or more guide molecules capable of hybridizing with one or more corresponding target sequences, and designed to form a CRISPR-Cas complex with the Cas, wherein, upon activation of the Cas by the binding of CRISPR-Cas complex to one or more of the target sequences, the Cas collateral activity cleaves the Cas-cleavable oligonucleotide linker(s), thereby generating a detectable luminescent signal indicating a presence of one or more of the target sequences in the sample. In an example embodiment, the Cas has collateral DNA cleavage activity, collateral RNA cleavage activity, or any combination thereof. In an example embodiment, the Cas having collateral cleavage activity is a Cas12, a Cas13, or any combination thereof.

### CRISPR-Cas Systems Overview

In general, a CRISPR-Cas or CRISPR-Cas system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein. Some CRISPR-Cas systems have collateral activity that is triggered by target recognition.

In some embodiments, the CRISPR-Cas system of the present composition can be a Class 1 system having collateral activity. The CRISPR-Cas system of the present composition can be a Class 2 system having collateral activity. The class 2 system may be a Type V or a Type VI CRISPR-Cas system.

### Type V

In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasY(Cas12d), CasX (Cas12e), Cas14, and/or Cas(D, homologues thereof, functional variants thereof, or modified versions thereof.

Cas12's non-specific cleavage can be leveraged to cleave reporters upon target recognition, allowing for the design of sensitive and specific diagnostics using Cas12, including single nucleotide variants, detection based on rRNA sequences, screening for drug resistance, monitoring microbe outbreaks, genetic perturbations, and screening of environmental samples, as described, for example, in Broughton et al. 2020. CRISPR-Cas12-based detection of SARS-CoV-2. Nat. Biotech. 38:870-874, https://doi.org/10.1038/s41587-020-0513-4; Leung et al. 2021. CRISPR-Cas12-based nucleic acids detection systems. Methods.; S1046-2023(21)00063-3.doi: 10.1016/j.ymeth.2021.02.018; Mahas et al., Viruses. 2021. 13:466, https://doi.org/10.3390/v13030466; Ali et al., 2020. iSCAN: An RT-LAMP-coupled CRISPR-Cas12 module for rapid, sensitive detection of SARS-CoV-2Vir. Res. 288:198129. https://doi.org/10.1016/j.virusres.2020.198129; Ramachandran et al., 2020. Electric field-driven microfluidics for rapid CRISPR-Cas basedCRISPR-Cas based diagnostics and its application to detection of SARS-CoV-2. PNAS Nov. 24, 2020 117 (47) 29518-29525; Mukama et al., An ultrasensitive and specific point-of care CRISPR-Cas12 based lateral flow biosensor for the rapid detection of nucleic acids. Biosens Bioelectron. 2020 Jul. 1; 159:112143. doi: 10.1016/j.bios.2020.112143; Chen et al., 2018. CRISPR-Cas12a target binding unleashes indiscriminate single-stranded DNase activity. Science. April 27; 360(6387):436-439. doi: 10.1126/science.aar6245; Kellner et al., 2019. Nat Protoc. 2019 October; 14(10):2986-3012. doi: 10.1038/s41596-019-0210-2; Broughton et al., 2020. Rapid Detection of 2019 Novel Coronavirus SARS-CoV-2 Using a CRISPR-based DETECTR Lateral Flow Assay. 2020. medRxiv. March 27; 2020.03.06.20032334. doi: 10.1101/2020.03.06.20032334; Wu et al. 2021. CRISPR-Cas12-Based Rapid Authentication of Halal Food. J Agric Food Chem. 2021 Aug. 26. doi: 10.1021/acs.jafc.1c03078; Long et al. 2021. CRISPR/Cas12-Based Ultra-Sensitive and Specific Point-of-Care Detection of HBV. Int J Mol Sci. 2021 May 3; 22(9):4842. doi: 10.3390/ijms22094842; Curti et al., Viruses. 2021 Mar. 5; 13(3):420. doi: 10.3390/v13030420; Li et al., Cell Discovery (2018)4:20. DOI 10.1038/s41421-018-0028-z; Lucia et al. 2020. An ultrasensitive, rapid, and portable coronavirus SARS-Cov-2 sequence detection method based on CRISPR-Cas12. bioRxiv preprint doi: https/doi.org/10.1101/2020.02.29.971127; MammothBiosciences. 2020. Broughton et al., available at https://mammoth.bio/wp-content/uploads/2020/04/200423-A-protocol-for-rapid-detection-of-SARS-CoV-2-using-CRISPR-diagnostics_3.pdf; East-Seletsky et al., Nat. 538:270, doi:10.1038/nature19802; International Pat. Pub. WO2019/233358; WO2019/011022; U.S. Pat. Nos. 10,337,051; 10,449,4664, 10,253,365; US 2020/0299768; US 2020/0399697; US 2019/0241954; the disclosure of each can be adapted for use with the present invention in view of the description provided herein and each of which is incorporated herein by reference in its entirety.

In some embodiments, the CRISPR-Cas system includes a Cas12b. In some embodiments, the Cas12b is an Alicyclobacillus acidoterrestris Cas12b (AacCas12b) or orthologue thereof. In some embodiments, the Cas 12b is an Cas12b from Alicyclobacillus acidiphilus (AapCas12b).

### Type VI

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d, homologues thereof, functional variants thereof, or modified versions thereof.

Type VI Cas's non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference; WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR-Cas based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26, 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6, Science. 2018 Apr. 27; 360(6387):439-444; Gootenberg J S, et al., Nucleic acid detection with CRISPR-Cas13a/C2c2, Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimizing deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, Zhang et al., “Two HPEN domains dictate CRISPR RNA maturation and target cleavage in Cas13d.” Nat. Comm. 10:2544 (2019), Patchsung et al., 2020. Nat. Biomed. Eng. 4:1140-1149; Aquino-Jarquin, G. Drug Discov. Today. 2021. 26(8):2025-2035; Fozouni et al., 2020. Amplification-free detection of SARS-CoV-2 with CRISPR-Cas13a and mobile phone microscopy. Cell. 184:323-333; Lotfi and Rezaei. 2020. CRISPR/Cas13: A potential therapeutic option of COVID-19 Biomedicine & Pharmacotherapy. 131:110738; Khan et al. 2020. CRISPR-Cas13 enzymology rapidly detects SARS-CoV-2 fragments in a clinical setting. medRxiv doi: https://doi.org/10.1101/2020.12.17.20228593; Schermer et al., Rapid SARS-CoV-2 testing in primary material based on a novel multiplex RT-LAMP assay. PLoS One. https://doi.org/10.1371/journal.pone.0238612; Joung et al., “Detection of SARS-CoV-2 with SHERLOCK One-Pot TestingN Engl J Med 2020; 383:1492-1494” DOI: 10.1056/NEJMc2026172; Joung et al., “Point-of-care testing for COVID-19 using SHERLOCK diagnostics” medRxiv. Preprint. 2020 May 8. doi: 10.1101/2020.05.04.20091231; WO 2017/218573; US 20200010878; US 20200010879; US 20190177775; US 20180208977; US 20180208976; US 20190177775; U.S. Provisional Application Ser. No. 62/351,172; the disclosure of each can be adapted for use with the present invention in view of the description provided herein and each of which is incorporated herein by reference in its entirety.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide molecule, e.g., RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

As used herein, the term “guide RNA” or “single guide RNA,” “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the gRNA and a CRISPR effector protein to the target nucleic acid sequence. In general, a gRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence. As used herein the term “capable of forming a complex with the CRISPR effector protein” refers to the gRNA having a structure that allows specific binding by the CRISPR effector protein to the gRNA such that a complex is formed that is capable of binding to a target RNA in a sequence specific manner and that can exert a function on said target RNA. Structural components of the gRNA may include direct repeats and a guide sequence (or spacer). The sequence specific binding to the target RNA is mediated by a part of the gRNA, the “guide sequence”, being complementary to the target RNA. In embodiments of the invention the term guide RNA, i.e. RNA capable of guiding Cas to a target locus, is used as in foregoing cited documents such as WO 2014/093622 (PCT/US2013/074667). As used herein the term “wherein the guide sequence is capable of hybridizing” refers to a subsection of the gRNA having sufficient complementarity to the target sequence to hybridize thereto and to mediate binding of a CRISPR complex to the target RNA. In general, a guide sequence is any polynucleotide sequence having sufficient complementarity with a target polynucleotide sequence to hybridize with the target sequence and direct sequence-specific binding of a CRISPR complex to the target sequence.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29 Jun. 2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

### Amplification Reagents

In an example embodiment, the detection systems described herein may further comprises nucleic acid amplification reagents. In an example embodiment, the nucleic acid amplification reagents comprise nucleic acid sequence-based amplification (NASBA) reagents, recombinase polymerase amplification (RPA) reagents, loop-mediated isothermal amplification (LAMP) reagents, strand displacement amplification (SDA) reagents, helicase-dependent amplification (HDA) reagents, nicking enzyme amplification reaction (NEAR) reagents, PCR reagents, multiple displacement amplification (MDA) reagents, rolling circle amplification (RCA) reagents, ligase chain reaction (LCR) reagents, or ramification amplification method (RAM) reagents.

### Methods of Use of Split-Luciferase Reporter Systems and Crispr-Cas Detection Systems Thereof

In an aspect, the present invention provides methods of detecting one or more target sequences in a sample. In an example embodiment, a method comprises: contacting a sample with any split-luciferase reporter CRISPR-Cas detection system of the present invention, wherein the generation of the detectable luminescent signal indicates the presence of one or more of the target sequences in the sample. In an example embodiment, the method further comprises detecting the detectable luminescent signal. In an example embodiment, the detecting step occurs for a period of time from 25 minutes to 3 hours. In an example embodiment, the sample is applied to a lateral flow immunochromatographic assay prior to or after the contacting occurs.

### Color-Coded Bead Multiplex Crispr-Cas Detection Systems

In another aspect, the present invention provides an improved multiplexed diagnostic system with increased sensitivity and deployability in resource constrained point-of-car-settings. The system comprises a CRISPR-Cas system liked those previously described above. A set of guide molecules is used to detect the presence of multiple targets in a sample in a single assay. Each guide molecule is covalently attached to a specific color-coded bead which identifies the guide molecule, and the corresponding target oligonucleotide to which the guide molecule is directed, by a specific color. The system also further comprises a detection construct that is configured to generate a detectable signal in the present of Cas collateral cleavage activity.

### Attachment of Guide Molecules to Color-Coded Beads

The systems and kits used in the methods described herein comprise one or more sets of guide molecules of the present invention. The sets of guide molecules can comprise multiple guide molecules, e.g., a panel of guide molecules, designed to bind to a target molecule. In an aspect, each set of guide molecules comprise guide molecules capable of binding one or more target sequences of a target molecule and designed to form a complex with a Cas protein. The guide molecules are distributed to individual discrete volumes, thereby spatially segregating each set of guide molecules. The individual discrete volumes are as defined elsewhere herein, and can comprise beads or wells in exemplary embodiments. Accordingly, the guide molecules can be provided on a bead, or in a well. The guide molecules may further comprise a first binding partner of a binding partner pair on the 5′ or the 3′ end. The binding partner may also be attached by hybridization of a complementary DNA sequence comprising a first binding partner of a partner pair to part of the spacer sequence of the guide molecule. The complementary DNA may comprise about 10 to about 20 nucleotides, preferably about 17 to 19 or about 18 nucleotides. The DNA sequence can comprise the first binding partner on the 3′ or the 5′ end of the DNA sequence. In an aspect, the guide is biotinylated, e.g., the first binding partner pair is biotin. The second binding partner of the binding partner pair is streptavidin and may be functionalized on a surface of the individual discrete volume, e.g., well or bead. In an aspect the binding partner pairs can contain a split luciferase (See, e.g., Yoshimura et al., Chem Rec. 14, 492-501 (2014) doi: 10.1002/tcr.201402001) or split lacZ reporter (See, e.g., Broome et al., Mol. Pharm. 2010 Feb. 1: 7(1):60-74; doi:10.1021/mp900188e), for example, with a first portion of the partner on the guide molecule and the second binding partner portion on the individual discrete volume.

### Beads

The beads used in the color-coded multiplex embodiments, may be the same of the same material and size as previously described above for use in the split-luciferase reporter embodiments, but with the additional feature of being color coded with various coloring dyes. Processes for coloring beads of the size and scale described herein are known in the art. In the present context, the bead may comprise guide molecules on any surface of the bead, including disposed within a porosity of the bead. In an embodiment, multiple sets of detection beads are provided, where each bead in a given set comprising guide molecules configured to detect a particular target molecule, and each different set of beads configured to detect a different target molecule such that detection of multiple target molecules is screened at once.

The bead can be further comprised or encapsulated in a droplet. In an embodiment, the detection bead is encapsulated in a first droplet and the reporter bead is encapsulated in a second droplet that can be subsequently merged into the same droplet.

The bead may be functionalized to permit covalent attachment of the guide molecules. Two beads of varying sizes and materials can be used; for example, a larger detection bead comprising guide molecules, and a smaller reporter bead comprising the reporter construct. Functionalization on the bead may comprise reactive groups that permit covalent attachment to the desired construct or guide molecule, and/or a label.

In one embodiment, the detection bead may be comprised in a droplet. In one example embodiment, a bead is provided and emulsified into droplets. The pooled beads can be emulsified into droplets via microfluidics or by shaking.

### Lyophilized Guides and Reagents

The guide molecules of the embodiments disclosed herein can be prepared in freeze-dried format for convenient distribution and point-of-care (POC) applications. Optionally one or more of Cas protein, reporter molecules, and detection reagents can also be freeze dried, or lyophilized, according to certain embodiments.

Each discrete volume may comprise a set of guide molecules (e.g., guide RNAs) for a different target molecule. These guide molecules (e.g., guide RNAs) may be specific for a target molecule but each guide molecule (e.g., guide RNA) in the set may vary, creating a panel of guide molecules (e.g., guide RNAs) for a target molecule. In certain embodiments, the CRISPR effector protein is bound or associated to each discrete volume. The Cas protein, the detection reagents, reporter molecules or a combination thereof can also be lyophilized. Exemplary methods of freeze-drying are described in International Patent Publication WO 2018/107129 at [0470], [0492], incorporated herein by reference. In an aspect, the lyophilized reagents are provided in a pre-decoded microwell array. In this manner, a sample can be provided, optionally pre-amplified, to the individual discrete volume. In particular instances, the sample is provided with one or more of the detection reagents, reporter molecules, or other components of the system, if not previously lyophilized and provided as part of the individual discrete volume. In a preferred embodiment, arrayed and encoded reagent sets are stored in the individual discrete volume. The encoded reagent sets may be provided in solution, on a bead, and/or lyophilized.

### Detection Constructs

In certain example embodiments, the composition further comprises a detection construct. The detection construct can be capable of producing one or more detectable signals. In certain example embodiments, the detection construct comprises an RNA or DNA oligonucleotide and further comprises a first molecule on a first end and a molecule on a second end. The detection construct can exist in an unmodified state and when modified by an activated effector of a CRISPR system, the detection construct can produce one or more detectable signals to indicate the presence of a target. In some embodiments, one or more of the detectable signals can be an assay control.

### Methods of Using Color-Coded Bead Multiplex Crispr-Cas Detection Systems

In an aspect, the present invention provides a color-coded bead multiplex CRISPR-Cas detection method for detecting a target sequence in a sample. In an example embodiment, the color-coded bead multiplex CRISPR-Cas detection method comprises: contacting a sample with any color-coded bead multiplex CRISPR-Cas detection system of the present invention, thereby forming a pre-mixture; mixing the pre-mixture with a solvent immiscible with the pre-mixture, thereby forming a pool of droplets comprising the sample and the detection CRISPR-Cas system; detecting each droplet generating a detectable signal and comprising a single one of the color-coded beads; and identifying the color-code of the single bead within said droplet, thereby indicating a presence in the sample of the target sequence corresponding to the guide molecule identified by the single color-coded bead in said droplet.

In an example embodiment, the pool of droplets is an oil-in-water emulsion. In an example embodiment, the oil is a fluorous oil. In an example embodiment, the oil further comprises a surfactant. In an example embodiment, the surfactant is a fluorinated surfactant. In an example embodiment, a color-coded bead multiplex CRISPR-Cas detection method further comprises, prior to the detecting steps, loading the droplet mixture onto a flow cell, and wherein the detecting steps are performed with a microscope. In an example embodiment, a color-coded bead multiplex CRISPR-Cas detection method further comprises, prior to the detecting steps, loading the droplet mixture onto a well plate, and wherein the detecting steps are performed with a plate reader.

### Kits

Any of the compounds, compositions, formulations, particles, cells, devices, and combinations thereof, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, dipsticks, substrates, bottles, and the like. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, devices, described herein or a combination thereof contained therein, safety information regarding the content of the compounds, compositions, formulations, particles, devices, and cells described herein or a combination thereof contained therein, information regarding the dosages, working amounts, indications for use, and/or recommended treatment regimen(s) for the compound(s) formulations, devices, and combinations thereof contained therein. In some embodiments, the instructions can provide directions for sample collection, sample preparation, and/or use of the compounds, compositions, formulations, particles, devices and cells described herein or a combination thereof. In some embodiments, the instructions can be specific to the target(s) being detected by a CRISPR effector detection system. In some embodiments, the instructions are specific to detecting a viral target, such as a viral polynucleotide. Exemplary virus that can be detected by the kits described herein are described elsewhere herein. In some embodiments, the viral target is SARS-CoV-2.

### Methods and Assays

Also described and provided herein are methods for detecting target nucleic acids in a sample. Such methods employ one or more of the CRISPR-Cas nucleic acid detection systems described herein, compositions described herein, and/or devices described herein. In general, the method includes optional amplification of one or more target sequences in a sample followed by detection of one or more amplified target sequences by a CRISPR-Cas collateral activity nucleic acid detection system and assay described herein. The target sequences can be present in a sample. In some embodiments, the sample is processed prior to amplification. Such processing can include lysis of one or more cells or virus or viral like particles present in the sample to release target nucleic acids. In some embodiments, the method does not require or include extraction of the nucleic acids from the sample prior to amplification and/or target detection. In some embodiments, the sample preparation (e.g., lysis) and amplification occur in the same reaction vessel or location. In some embodiments, the sample preparation (e.g., lysis), target amplification, and CRISPR-Cas based nucleic acid detection occur in the same reaction vessel or location. In some embodiments, the reaction vessel or location contains the sample preparation, amplification, and/or CRISPR-Cas detection compositions and/or systems. In these embodiments, the sample can be added to the vessel and processing, amplification and detection can occur in the same vessel with no requirement to remove or add reagents to the vessel prior to obtaining a result. In some embodiments, the reagents, compositions, and systems are included in a vessel in a dehydrated (e.g., freeze dried, lyophilized, etc.) form and can be reconstituted when ready to use. In some embodiments, the processing (e.g., lysis, amplification, and/or CRISPR-Cas nucleic acid detection) can be performed at ambient or at about body temperature.

In some embodiments, the method can employ a Cas13 or Cas12 CRISPR-Cas system for target nucleic acid detection. See e.g., Jong et al. N Engl J Med. 2020. 383(15):1492-1494; Broughton, et al. CRISPR-Cas12-based detection of SARS-CoV-2. Nat Biotechnol (2020), doi:10.1038/s41587-020-0513-4 (DETECTR detection); Gootenberg et al., Science. 2018 Apr. 27; 360(6387):439-444. doi: 10.1126/science.aaq0179 (multiplexing lateral flow platform for point-of-care diagnostics); and Chen, et al., Science. 2018 Apr. 27; 360(6387):436-439. doi: 10.1126/science.aar6245 (Cas12 detection), each of which is incorporated by reference. Similarly, data from field deployable technologies can be utilized in accordance with the present invention. See, Myrhvold et al., Science 27 Apr. 2018: 360:6387, pp. 444-448; doi:10.1126/science.aas8836 (field deployable viral diagnostics), which are incorporated herein by herein by reference. Point-of-care testing is a preferred data source and may include population-scale diagnostics. See, e.g. Joung et al., Point-of-care testing for COVID-19 using SHERLOCK diagnostics” doi: 10.1101/2020.05.04.20091231; Schmid-Burgk, et al., “LAMP-Seq: Population-Scale COVID-19 Diagnostics Using Combinatorial Barcoding,” doi: 10.1101/2020.04.06.025635, each of which is incorporated herein by reference.

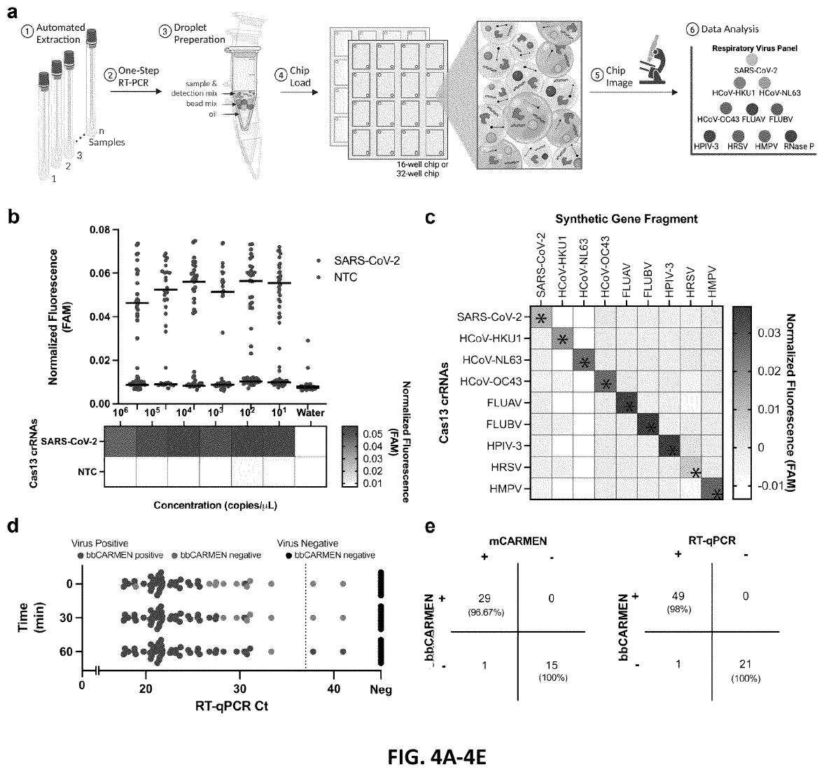

Certain example embodiments disclosed herein provide are based on low-cost CRISPR-Cas based diagnostic that enables single-molecule detection of DNA or RNA with single-nucleotide specificity (Gootenberg, 2018; Gootenberg, et al, Science. 2017 Apr. 28; 356(6336):438-442 (2017); Myhrvold, et al., Science 360, 444-448 (2018)). Nucleic acid detection with SHERLOCK relies on the collateral activity of Type VI and Type V Cas proteins, such as Cas13 and Cas12, which unleashes promiscuous cleavage of reporters upon target detection (Gooteneberg et al., 2018) (Abudayyeh, et al., Science. 353(6299)(2016); East-Seletsky et al. Nature 538:270-273 (2016); Smargon et al. Mol Cell 65(4):618-630 (2017)). Certain embodiments disclosed herein, are capable of single-molecule detection in less than an hour and can be used for multiplexed target detection when using CRISPR enzymes with orthogonal cleavage preference, such as Cas13a from Leptotrichia wadei (LwaCas13a), Cas13b from Capnocytophaga canimorsus Cc5 (CcaCas13b), and Cas12a from Acidaminococcus sp. BV3L6 (AsCas12a); Alicyclobacillus acidiphilus (Aap) Cas 12b and Brevibacillus sp. SYSU G02855 (BrCas12b); (Gootenberg, 2018; Myhrvold et al. Science 360(6387):444-448 (2018); Gootenberg, 2017; Chen et al. Science 360(6387):436-439 (2018); Li et al. Cell Rep 25(12):3262-3272 (2018); Li et al. Nat Protoc 13(5):899-914 (2018)). Guide molecules used herein are designed using a model for high activity-based Cas guide selection for coronavirus would facilitate design of optimal diagnostic assays, especially in applications requiring high-activity guides like lateral flow detection, and enable guide RNA design for in vivo RNA targeting applications with Cas13 has also been detailed in U.S. Provisional Applications 62/818,702 filed Mar. 14, 2019, now PCT/US20/22795 and 62/890,555, filed Aug. 22, 2019, now PCT/US20/22795, both entitled CRISPR Effector System Based Multiplex Diagnostics, incorporated herein by reference in their entirety, and, in particular, Examples 1-4, Tables 1-8 and FIG. 4A of U.S. Provisional Application 62/890,555.

The low cost and adaptability of the assay platform described herein lends itself to a number of applications including (i) general viral RNA/DNA quantitation, (ii) rapid, multiplexed RNA/DNA expression detection, and (iii) sensitive detection of target nucleic acids in both clinical and environmental samples. Additionally, the systems disclosed herein may be adapted for detection of transcripts within biological settings, such as cells. Given the highly specific nature of the CRISPR effectors described herein, it may be possible to track allelic specific expression of transcripts or disease-associated mutations and/or the presence of microorganisms in live cells.

In certain example embodiments, a single guide RNA specific to a single target is placed in separate volumes. Each volume may then receive a different sample or aliquot of the same sample. In certain example embodiments, multiple guide RNA each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, U, or T. Accordingly, guide RNAs that are all, or comprise a substantial portion, of a single nucleotide may be generated, each with a different fluorophore. In this way up to four different targets may be screened in a single individual discrete volume.

Generally, the CRISPR effector system detection method can be composed of two parts: 1) sample preparation and 2) CRISPR effector system detection of one or more targets present in the sample. The CRISPR effector system detection portion of the method can include a transcription step followed by CRISPR-effector system mediated detection of a target. In some embodiments, the CRISPR effector system detection portion of the method can also include target amplification and/or signal amplification/enrichment. These steps are described in greater detail below and elsewhere herein. In some embodiments, one or more of the steps within each of the portions of the method are performed in the same reaction vessel, reaction area/location, and/or device. In some embodiments all of the steps of the method are performed in the same reaction vessel, same reaction vessel, reaction area/location, and/or device.

In some embodiments, the CRISPR effector systems and methods herein are capable of detecting down to at least attomolar concentrations of target molecules, such as viral polynucleotides. In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL). In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL) using a fluorescent or colorimetric readout.

In some embodiments, the polynucleotides are released from cells in the sample and the CRISRP-effector system detection can occur on the released polynucleotides without extracting the sample polynucleotides from other components in the sample. This can allow for the sample preparation and CRISRP-effector detection reaction to be performed in the same reaction vessel. 0128 In some embodiments, one or more or all of the steps included in the CRISPR-effector system detection reaction can occur at about 22-55 degrees C. (including any target and/or signal amplification). In some embodiments, one or more or all of the steps included in the CRISPR-effector system detection reaction can occur at about 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, to/or 55 degrees C., 22, 23, 24, 25, 26, 27, 28, 29, 30, 31, 32, 33, 34, 35, 36, to/or 37 degrees C., about 22, 23, 24, 25, 26, 27, 28, 29, 30, 31, 32, or/to 33 degrees C., about 22, 23, 24, 25, 26, or/to 27 degrees C., or about 22, 23, 24, to/or 25 degrees C. In some embodiments, one or more or all of the steps included in the CRISPR-effector system detection reaction can occur at about room temperature (about 22-25 degrees C.).

In some embodiments, the CRISPR-effector system detection reaction can occur as a two-step reaction in which amplification of target(s) and target detection via the CRISPR effector system occur in separate reactions. In some embodiments, The CRISPR-effector system detection reaction (including any target and/or signal amplification) can occur as a single, one-pot reaction. In some embodiments where the CRISPR-effector system detection reaction is a one-pot reaction, target amplification is achieved using LAMP or RPA. In some embodiments where the CRISRP-effector system detection reaction is a one-pot reaction the CRISPR-effector system includes a Cas 12 (such as a Cas12b) or a Cas13 (such as a cas13a). In some embodiments where the CRISRP-effector system detection reaction is a one-pot reaction and target amplification is achieved using LAMP, the CRISPR-effector system includes a Cas12, such as a Cas12b. In some embodiments where the CRISRP-effector system detection reaction is a one-pot reaction and target amplification is achieved using RPA, the CRISPR-effector system includes a Cas13, such as a Cas13a. In some embodiments, sample preparation and a single, one-pot CRISPR effector system can occur in the same reaction vessel, thus eliminating the need to move potentially hazardous samples from one reaction vessel to another.

In some embodiments, the total time to perform the CRISPR-effector system detection method (from sample preparation to detection) can be greater than 0 hours but less than about 4, 3.5, 3, 2.5, 2, 1.5, 1, or 0.5 hours. In some embodiments, the total time to perform the CRISPR-effector system detection method (from sample preparation to detection) can occur within about 20 to 120 minutes, such as within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, to/or 120 minutes. In some embodiments, the total time to perform the CRISPR-effector system detection method (from sample preparation to detection) can occur within about 20 to about 60 minutes, e.g. within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or/to 60 minutes. In some embodiments, the total time to perform the CRISPR-effector system detection method (from sample preparation to detection) can occur within about 20 to about 45 minutes, e.g. within about 20, 21, 22, 23, 24, 25, 26, 27, 28, 29, 30, 31, 32, 33, 34, 35, 36, 37, 38, 39, 40, 41, 42, 43, 44, and/or 45 minutes. In some embodiments, the total time to perform the CRISPR-effector system detection method (from sample preparation to detection) can occur within about 20 to about 30 minutes, e.g., within about 20, 21, 22, 23, 24, 25, 26, 27, 28, 29, and/or 30 minutes.

In some embodiments, the CRISPR-effector system detection reaction can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the CRISPR-effector system detection reaction can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the CRISPR-effector system detection reaction can occur within about 1 to about 30 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the CRISPR-effector system detection reaction can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the CRISPR-effector system detection reaction can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the CRISPR-effector system detection reaction can occur within about 1 to about 15 minutes, e.g., within about 1, 2, 3, 4, 5, 6, 7, 8, 9, 10, 11, 12, 13, to/or about 15 minutes. In some embodiments, the CRISPR-effector system detection reaction can occur within about 1 to about 10 minutes, e.g., within about 1, 2, 3, 4, 5, 6, 7, 8, 9, to/or about 10 minutes. In some embodiments, the CRISPR-effector system detection reaction can occur within about 1 to about 5 minutes, e.g., within about 1, 2, 3, 4, to/or about 5 minutes.

In some embodiments, the method includes preparation of the reagents for one or more steps, such as sample preparation, amplification, and/or CRISPR/Cas detection, for storage. Such storage preparation can include, but is not limited to lyophilizing, freeze drying, or otherwise dehydrating them. They can be prepared for storage inside of individual reaction vessels or locations within a device or other vessel. In some of these embodiments, the reagents, compositions, systems or combinations thereof are e.g., lyophilized or freeze dried inside of the reaction vessel or at the specific discreet locations on a substrate or otherwise in a device. They can be stored at a temperature ranging from ambient temperature (e.g., about 25-32 degrees C.) to about −20 or −80 degrees Celsius. In some embodiments, they are stored for 1, 2, 3, 4, 5, 6, 7, 8, 9, 10, 11, or 12 days, weeks, months or years. In some embodiments, the reagents, compositions, systems or combinations thereof are prepared and stored at about 4 degrees C. for about 1, 2, 3, 4, 5, 6, 7, 8, 9, 10, 11, or 12 days, weeks, months or years or more.

Due to the sensitivity of said systems, a number of applications that require from the rapid and sensitive detection may benefit from the embodiments disclosed herein and are contemplated to be within the scope of the invention.

In an aspect, the embodiments disclosed herein are directed to methods for detecting target nucleic acids in a sample. The methods disclosed herein can, in some embodiments, comprise steps of generating a first set of droplets, each droplet in the first set of droplets comprising at least one target molecule and an optical barcode; generating a second set of droplets, each droplet in the second set of droplets comprising a detection CRISPR system comprising an RNA or DNA targeting effector protein and one or more guide molecules (e.g., guide RNAs) designed to bind to corresponding target molecules, an oligonucleotide detection construct (e.g., an RNA-based or DNA-based detection construct) and optionally an optical barcode; combining the first set and second set of droplets into a pool of droplets and flowing the combined pool of droplets onto a microfluidic device comprising an array of microwells and at least one flow channel beneath the microwells, the microwells sized to capture at least two droplets; capturing droplets in the microwell and detecting the optical barcodes of the droplets captured in each microwell; merging the droplets captured in each microwell to formed merged droplets in each microwell, at least a subset of the merged droplets comprising a detection CRISPR system and a target sequence; initiating the detection reaction. The merged droplets are then maintained under conditions sufficient to allow binding of the one or more guide molecules (e.g., guide RNAs) to one or more target molecules. Binding of the one or more guide RNAs to a target nucleic acid in turn activates the CRISPR effector protein. Once activated, the CRISPR effector protein then deactivates the masking construct, for example, by cleaving the masking construct such that a detectable positive signal is unmasked, released, or generated. Detection and measuring a detectable signal of each merged droplet at one or more time periods can be performed, indicating the presence of target molecules when, for example the positive detectable signal is present.

In particular embodiments, the systems are highly targeted for single samples such that an optical barcode in a second set of barcodes is not needed, or is optional. In certain embodiments, advanced, improved, or more powerful preamplification methods allow omission of an optical barcode in a set of the droplets. Accordingly, optical barcodes in a set of droplets are optional, and inclusion can depend on the particular application, including sample quality, target specificity, preamplification techniques, among other variables.

### Droplets

The droplets as provided herein are typically water-in-oil microemulsions formed with an oil input channel and an aqueous input channel. The droplets can be formed by a variety of dispersion methods known in the art. In one particular embodiment, a large number of uniform droplets in oil phase can be made by microemulsion. Exemplary methods can include, for example, R-junction geometry where an aqueous phase is sheared by oil and thereby generates droplets; flow-focusing geometry where droplets are produced by shearing the aqueous stream from two directions; or co-flow geometry where an aqueous phase is ejected through a thin capillary, placed coaxially inside a bigger capillary through which oil is pumped.

The use of monodisperse aqueous droplets can be generated by a microfluidic device as a water-in-oil emulsion. In one embodiment, the droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules.

The aqueous droplets of volume ranging from 1 pL to 10 nL work as individual reactors. 104 to 105 single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length are selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled.

In one arrangement, the expansion and/or contraction of liquid reservoirs may be used to steer or sort a fluidic droplet into a channel, e.g., by causing directed movement of the liquid containing the fluidic droplet. In another, the expansion and/or contraction of the liquid reservoir may be combined with other flow-controlling devices and methods, e.g., as described herein. Non-limiting examples of devices able to cause the expansion and/or contraction of a liquid reservoir include pistons. Key elements for using microfluidic channels to process droplets include: (1) producing droplet of the correct volume, (2) producing droplets at the correct frequency and (3) bringing together a first stream of sample droplets with a second stream of sample droplets in such a way that the frequency of the first stream of sample droplets matches the frequency of the second stream of sample droplets. Preferably, bringing together a stream of sample droplets with a stream of premade library droplets in such a way that the frequency of the library droplets matches the frequency of the sample droplets. Methods for producing droplets of a uniform volume at a regular frequency are well known in the art. One method is to generate droplets using hydrodynamic focusing of a dispersed phase fluid and immiscible carrier fluid, such as disclosed in U.S. Publication No. US 2005/0172476 and International Publication No. WO 2004/002627. It is desirable for one of the species introduced at the confluence to be a pre-made library of droplets where the library contains a plurality of reaction conditions, e.g., a library may contain plurality of different compounds at a range of concentrations encapsulated as separate library elements for screening their effect on cells or enzymes, alternatively a library could be composed of a plurality of different primer pairs encapsulated as different library elements for targeted amplification of a collection of loci, alternatively a library could contain a plurality of different antibody species encapsulated as different library elements to perform a plurality of binding assays. The introduction of a library of reaction conditions onto a substrate is achieved by pushing a premade collection of library droplets out of a vial with a drive fluid. The drive fluid is a continuous fluid. The drive fluid may comprise the same substance as the carrier fluid (e.g., a fluorocarbon oil). For example, if a library consists of ten pico-liter droplets is driven into an inlet channel on a microfluidic substrate with a drive fluid at a rate of 10,000 pico-liters per second, then nominally the frequency at which the droplets are expected to enter the confluence point is 1000 per second. However, in practice droplets pack with oil between them that slowly drains. Over time the carrier fluid drains from the library droplets and the number density of the droplets (number/mL) increases. Hence, a simple fixed rate of infusion for the drive fluid does not provide a uniform rate of introduction of the droplets into the microfluidic channel in the substrate. Moreover, library-to-library variations in the mean library droplet volume result in a shift in the frequency of droplet introduction at the confluence point. Thus, the lack of uniformity of droplets that results from sample variation and oil drainage provides another problem to be solved. For example if the nominal droplet volume is expected to be 10 pico-liters in the library, but varies from 9 to 11 pico-liters from library-to-library then a 10,000 pico-liter/second infusion rate will nominally produce a range in frequencies from 900 to 1,100 droplet per second. In short, sample to sample variation in the composition of dispersed phase for droplets made on chip, a tendency for the number density of library droplets to increase over time and library-to-library variations in mean droplet volume severely limit the extent to which frequencies of droplets may be reliably matched at a confluence by simply using fixed infusion rates. In addition, these limitations also have an impact on the extent to which volumes may be reproducibly combined. Combined with typical variations in pump flow rate precision and variations in channel dimensions, systems are severely limited without a means to compensate on a run-to-run basis. The foregoing facts not only illustrate a problem to be solved, but also demonstrate a need for a method of instantaneous regulation of microfluidic control over microdroplets within a microfluidic channel.

Combinations of surfactant(s) and oils must be developed to facilitate generation, storage, and manipulation of droplets to maintain the unique chemical/biochemical/biological environment within each droplet of a diverse library. Therefore, the surfactant and oil combination should (1) stabilize droplets against uncontrolled coalescence during the drop forming process and subsequent collection and storage, (2) minimize transport of any droplet contents to the oil phase and/or between droplets, and (3) maintain chemical and biological inertness with contents of each droplet (e.g., no adsorption or reaction of encapsulated contents at the oil-water interface, and no adverse effects on biological or chemical constituents in the droplets). In addition to the requirements on the droplet library function and stability, the surfactant-in-oil solution must be coupled with the fluid physics and materials associated with the platform. Specifically, the oil solution must not swell, dissolve, or degrade the materials used to construct the microfluidic chip, and the physical properties of the oil (e.g., viscosity, boiling point, etc.) must be suited for the flow and operating conditions of the platform. Droplets formed in oil without surfactant are not stable to permit coalescence, so surfactants must be dissolved in the oil that is used as the continuous phase for the emulsion library. Surfactant molecules are amphiphilic—part of the molecule is oil soluble, and part of the molecule is water soluble. When a water-oil interface is formed at the nozzle of a microfluidic chip for example in the inlet module described herein, surfactant molecules that are dissolved in the oil phase adsorb to the interface. The hydrophilic portion of the molecule resides inside the droplet and the fluorophilic portion of the molecule decorates the exterior of the droplet. The surface tension of a droplet is reduced when the interface is populated with surfactant, so the stability of an emulsion is improved. In addition to stabilizing the droplets against coalescence, the surfactant should be inert to the contents of each droplet and the surfactant should not promote transport of encapsulated components to the oil or other droplets. A droplet library may be made up of a number of library elements that are pooled together in a single collection (see, e.g., US Patent Publication No. 2010002241).

Libraries may vary in complexity from a single library element to 1015 library elements or more. Each library element may be one or more given components at a fixed concentration. The element may be, but is not limited to, cells, organelles, virus, bacteria, yeast, beads, amino acids, proteins, polypeptides, nucleic acids, polynucleotides or small molecule chemical compounds. The element may contain an identifier such as a label. The terms “droplet library” or “droplet libraries” are also referred to herein as an “emulsion library” or “emulsion libraries.” These terms are used interchangeably throughout the specification. A cell library element may include, but is not limited to, hybridomas, B-cells, primary cells, cultured cell lines, cancer cells, stem cells, cells obtained from tissue, or any other cell type. Cellular library elements are prepared by encapsulating a number of cells from one to hundreds of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picolitre drops.” Lab Chip, 8(8): 1262-1264, 2008. The discrete nature of cells allows for libraries to be prepared in mass with a plurality of cellular variants all present in a single starting media and then that media is broken up into individual droplet capsules that contain at most one cell. These individual droplets capsules are then combined or pooled to form a library consisting of unique library elements. Cell division subsequent to, or in some embodiments following, encapsulation produces a clonal library element.

In certain embodiments, a bead based library element may contain one or more beads, of a given type and may also contain other reagents, such as antibodies, enzymes or other proteins. In the case where all library elements contain different types of beads, but the same surrounding media, the library elements may all be prepared from a single starting fluid or have a variety of starting fluids. In the case of cellular libraries prepared in mass from a collection of variants, such as genomically modified, yeast or bacteria cells, the library elements will be prepared from a variety of starting fluids. Often it is desirable to have exactly one cell per droplet with only a few droplets containing more than one cell when starting with a plurality of cells or yeast or bacteria, engineered to produce variants on a protein. In some cases, variations from Poisson statistics may be achieved to provide an enhanced loading of droplets such that there are more droplets with exactly one cell per droplet and few exceptions of empty droplets or droplets containing more than one cell. Examples of droplet libraries are collections of droplets that have different contents, ranging from beads, cells, small molecules, DNA, primers, antibodies. Smaller droplets may be in the order of femtoliter (fL) volume drops, which are especially contemplated with the droplet dispensors. The volume may range from about 5 to about 600 fL. The larger droplets range in size from roughly 0.5 micron to 500 micron in diameter, which corresponds to about 1 pico liter to 1 nano liter. However, droplets may be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges. The droplets within the emulsion libraries of the present invention may be contained within an immiscible oil which may comprise at least one fluorosurfactant. In some embodiments, the fluorosurfactant within the immiscible fluorocarbon oil may be a block copolymer consisting of one or more perfluorinated polyether (PFPE) blocks and one or more polyethylene glycol (PEG) blocks. In other embodiments, the fluorosurfactant is a triblock copolymer consisting of a PEG center block covalently bound to two PFPE blocks by amide linking groups. The presence of the fluorosurfactant (similar to uniform size of the droplets in the library) is critical to maintain the stability and integrity of the droplets and is also essential for the subsequent use of the droplets within the library for the various biological and chemical assays described herein. Fluids (e.g., aqueous fluids, immiscible oils, etc.) and other surfactants that may be utilized in the droplet libraries of the present invention are described in greater detail herein.

The present invention can accordingly involve an emulsion library which may comprise a plurality of aqueous droplets within an immiscible oil (e.g., fluorocarbon oil) which may comprise at least one fluorosurfactant, wherein each droplet is uniform in size and may comprise the same aqueous fluid and may comprise a different library element. The present invention also provides a method for forming the emulsion library which may comprise providing a single aqueous fluid which may comprise different library elements, encapsulating each library element into an aqueous droplet within an immiscible fluorocarbon oil which may comprise at least one fluorosurfactant, wherein each droplet is uniform in size and may comprise the same aqueous fluid and may comprise a different library element, and pooling the aqueous droplets within an immiscible fluorocarbon oil which may comprise at least one fluorosurfactant, thereby forming an emulsion library. For example, in one type of emulsion library, all different types of elements (e.g., cells or beads), may be pooled in a single source contained in the same medium. After the initial pooling, the cells or beads are then encapsulated in droplets to generate a library of droplets wherein each droplet with a different type of bead or cell is a different library element. The dilution of the initial solution enables the encapsulation process. In some embodiments, the droplets formed will either contain a single cell or bead or will not contain anything, i.e., be empty. In other embodiments, the droplets formed will contain multiple copies of a library element. The cells or beads being encapsulated are generally variants on the same type of cell or bead. In another example, the emulsion library may comprise a plurality of aqueous droplets within an immiscible fluorocarbon oil, wherein a single molecule may be encapsulated, such that there is a single molecule contained within a droplet for every 20-60 droplets produced (e.g., 20, 25, 30, 35, 40, 45, 50, 55, 60 droplets, or any integer in between). Single molecules may be encapsulated by diluting the solution containing the molecules to such a low concentration that the encapsulation of single molecules is enabled. Formation of these libraries may rely on limiting dilutions.

The present invention also provides an emulsion library which may comprise at least a first aqueous droplet and at least a second aqueous droplet within an oil, in one embodiment a fluorocarbon oil, which may comprise at least one surfactant, in one embodiment a fluorosurfactant, wherein the at least first and the at least second droplets are uniform in size and comprise a different aqueous fluid and a different library element. The present invention also provides a method for forming the emulsion library which may comprise providing at least a first aqueous fluid which may comprise at least a first library of elements, providing at least a second aqueous fluid which may comprise at least a second library of elements, encapsulating each element of said at least first library into at least a first aqueous droplet within an immiscible fluorocarbon oil which may comprise at least one fluorosurfactant, encapsulating each element of said at least second library into at least a second aqueous droplet within an immiscible fluorocarbon oil which may comprise at least one fluorosurfactant, wherein the at least first and the at least second droplets are uniform in size and may comprise a different aqueous fluid and a different library element, and pooling the at least first aqueous droplet and the at least second aqueous droplet within an immiscible fluorocarbon oil which may comprise at least one fluorosurfactant thereby forming an emulsion library.

One of skill in the art will recognize that methods and systems of the invention need not be limited to any particular type of sample, and methods and systems of the invention may be used with any type of organic, inorganic, or biological molecule (see, e.g, US Patent Publication No. 20120122714).

In particular embodiments the sample may include nucleic acid target molecules. Nucleic acid molecules may be synthetic or derived from naturally occurring sources. In one embodiment, nucleic acid molecules may be isolated from a biological sample containing a variety of other components, such as proteins, lipids and non-template nucleic acids. Nucleic acid target molecules may be obtained from any cellular material, obtained from an animal, plant, bacterium, fungus, or any other cellular organism. In certain embodiments, the nucleic acid target molecules may be obtained from a single cell. Biological samples for use in the present invention may include viral particles or preparations. Nucleic acid target molecules may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid target molecules may also be isolated from cultured cells, such as a primary cell culture or a cell line. The cells or tissues from which target nucleic acids are obtained may be infected with a virus or other intracellular pathogen. A sample may also be total RNA extracted from a biological specimen, a cDNA library, viral, or genomic DNA. Generally, nucleic acid may be extracted from a biological sample by a variety of techniques such as those described by Maniatis, et al., Molecular Cloning: A Laboratory Manual, Cold Spring Harbor, N.Y., pp. 280-281 (1982). Nucleic acid molecules may be single-stranded, double-stranded, or double-stranded with single-stranded regions (for example, stem- and loop-structures). Nucleic acid obtained from biological samples typically may be fragmented to produce suitable fragments for analysis. Target nucleic acids may be fragmented or sheared to desired length, using a variety of mechanical, chemical and/or enzymatic methods. DNA may be randomly sheared via sonication, e.g. Covaris method, brief exposure to a DNase, or using a mixture of one or more restriction enzymes, or a transposase or nicking enzyme. RNA may be fragmented by brief exposure to an RNase, heat plus magnesium, or by shearing. The RNA may be converted to cDNA. If fragmentation is employed, the RNA may be converted to cDNA before or after fragmentation. In one embodiment, nucleic acid from a biological sample is fragmented by sonication. In another embodiment, nucleic acid is fragmented by a hydroshear instrument. Generally, individual nucleic acid target molecules may be from about 40 bases to about 40 kb. Nucleic acid molecules may be single-stranded, double-stranded, or double-stranded with single-stranded regions (for example, stem- and loop-structures). A biological sample as described herein may be homogenized or fractionated in the presence of a detergent or surfactant. The concentration of the detergent in the buffer may be about 0.05% to about 10.0%. The concentration of the detergent may be up to an amount where the detergent remains soluble in the solution. In one embodiment, the concentration of the detergent is between 0.1% to about 2%. The detergent, particularly a mild one that is nondenaturing, may act to solubilize the sample. Detergents may be ionic or nonionic. Examples of nonionic detergents include triton, such as the Triton™ X series (Triton™ X-100 t-Oct-C6H4-(OCH2-CH2)xOH, x=9-10, Triton™ X-100R, Triton™ X-114 x=7-8), octyl glucoside, polyoxyethylene(9)dodecyl ether, digitonin, IGEPAL™ CA630 octylphenyl polyethylene glycol, n-octyl-beta-D-glucopyranoside (betaOG), n-dodecyl-beta, Tween™. 20 polyethylene glycol sorbitan monolaurate, Tween™ 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14E06), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant. Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid. Size selection of the nucleic acids may be performed to remove very short fragments or very long fragments. The nucleic acid fragments may be partitioned into fractions which may comprise a desired number of fragments using any suitable method known in the art. Suitable methods to limit the fragment size in each fragment are known in the art. In various embodiments of the invention, the fragment size is limited to between about 10 and about 100 Kb or longer. A sample in or as to the instant invention may include individual target proteins, protein complexes, proteins with translational modifications, and protein/nucleic acid complexes. Protein targets include peptides, and also include enzymes, hormones, structural components such as viral capsid proteins, and antibodies. Protein targets may be synthetic or derived from naturally-occurring sources. The invention protein targets may be isolated from biological samples containing a variety of other components including lipids, non-template nucleic acids, and nucleic acids. Protein targets may be obtained from an animal, bacterium, fungus, cellular organism, and single cells. Protein targets may be obtained directly from an organism or from a biological sample obtained from the organism, including bodily fluids such as blood, urine, cerebrospinal fluid, seminal fluid, saliva, sputum, stool and tissue. Protein targets may also be obtained from cell and tissue lysates and biochemical fractions. An individual protein is an isolated polypeptide chain. A protein complex includes two or polypeptide chains. Samples may include proteins with post translational modifications including but not limited to phosphorylation, methionine oxidation, deamidation, glycosylation, ubiquitination, carbamylation, s-carboxymethylation, acetylation, and methylation. Protein/nucleic acid complexes include cross-linked or stable protein-nucleic acid complexes. Extraction or isolation of individual proteins, protein complexes, proteins with translational modifications, and protein/nucleic acid complexes is performed using methods known in the art.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. The present invention may relate to systems and methods for manipulating droplets within a high-throughput microfluidic system. A microfluid droplet may encapsulate a differentiated cell, the cell is lysed and its mRNA is hybridized onto a capture bead containing barcoded oligo dT primers on the surface, all inside the droplet. The barcode is covalently attached to the capture bead via a flexible multi-atom linker like PEG. In a preferred embodiment, the droplets are broken by addition of a fluorosurfactant (like perfluorooctanol), washed, and collected. A reverse transcription (RT) reaction is then performed to convert each cell's mRNA into a first strand cDNA that is both uniquely barcoded and covalently linked to the mRNA capture bead. Subsequently, a universal primer via a template switching reaction is amended using conventional library preparation protocols to prepare an RNA-Seq library. Since all of the mRNA from any given cell is uniquely barcoded, a single library is sequenced and then computationally resolved to determine which mRNAs came from which cells. In this way, through a single sequencing run, tens of thousands (or more) of distinguishable transcriptomes can be simultaneously obtained. The oligonucleotide sequence may be generated on the bead surface. During these cycles, beads were removed from the synthesis column, pooled, and aliquoted into four equal portions by mass; these bead aliquots were then placed in a separate synthesis column and reacted with either dG, dC, dT, or dA phosphoramidite. In other instances, dinucleotide, trinucleotides, or oligonucleotides that are greater in length are used, in other instances, the oligo-dT tail is replaced by gene specific oligonucleotides to prime specific targets (singular or plural), random sequences of any length for the capture of all or specific RNAs. This process was repeated 12 times for a total of 412=16,777,216 unique barcode sequences. Upon completion of these cycles, 8 cycles of degenerate oligonucleotide synthesis were performed on all the beads, followed by 30 cycles of dT addition. In other embodiments, the degenerate synthesis is omitted, shortened (less than 8 cycles), or extended (more than 8 cycles); in others, the 30 cycles of dT addition are replaced with gene specific primers (single target or many targets) or a degenerate sequence. The aforementioned microfluidic system is regarded as the reagent delivery system microfluidic library printer or droplet library printing system of the present invention. Droplets are formed as sample fluid flows from droplet generator which contains lysis reagent and barcodes through microfluidic outlet channel which contains oil, towards junction. Defined volumes of loaded reagent emulsion, corresponding to defined numbers of droplets, are dispensed on-demand into the flow stream of carrier fluid. The sample fluid may typically comprise an aqueous buffer solution, such as ultrapure water (e.g., 18 mega-ohm resistivity, obtained, for example by column chromatography), 10 mM Tris HCl and 1 mM EDTA (TE) buffer, phosphate buffer saline (PBS) or acetate buffer. Any liquid or buffer that is physiologically compatible with nucleic acid molecules can be used. The carrier fluid may include one that is immiscible with the sample fluid. The carrier fluid can be a non-polar solvent, decane (e.g., tetradecane or hexadecane), fluorocarbon oil, silicone oil, an inert oil such as hydrocarbon, or another oil (for example, mineral oil). The carrier fluid may contain one or more additives, such as agents which reduce surface tensions (surfactants). Surfactants can include Tween, Span, fluorosurfactants, and other agents that are soluble in oil relative to water. In some applications, performance is improved by adding a second surfactant to the sample fluid. Surfactants can aid in controlling or optimizing droplet size, flow and uniformity, for example by reducing the shear force needed to extrude or inject droplets into an intersecting channel. This can affect droplet volume and periodicity, or the rate or frequency at which droplets break off into an intersecting channel. Furthermore, the surfactant can serve to stabilize aqueous emulsions in fluorinated oils from coalescing. Droplets may be surrounded by a surfactant which stabilizes the droplets by reducing the surface tension at the aqueous oil interface. Preferred surfactants that may be added to the carrier fluid include, but are not limited to, surfactants such as sorbitan-based carboxylic acid esters (e.g., the “Span” surfactants, Fluka Chemika), including sorbitan monolaurate (Span 20), sorbitan monopalmitate (Span 40), sorbitan monostearate (Span 60) and sorbitan monooleate (Span 80), and perfluorinated polyethers (e.g., DuPont Krytox 157 FSL, FSM, and/or FSH). Other non-limiting examples of non-ionic surfactants which may be used include polyoxyethylenated alkylphenols (for example, nonyl-, p-dodecyl-, and dinonylphenols), polyoxyethylenated straight chain alcohols, polyoxyethylenated polyoxypropylene glycols, polyoxyethylenated mercaptans, long chain carboxylic acid esters (for example, glyceryl and polyglyceryl esters of natural fatty acids, propylene glycol, sorbitol, polyoxyethylenated sorbitol esters, polyoxyethylene glycol esters, etc.) and alkanolamines (e.g., diethanolamine-fatty acid condensates and isopropanolamine-fatty acid condensates). In some cases, an apparatus for creating a single-cell sequencing library via a microfluidic system provides for volume-driven flow, wherein constant volumes are injected over time. The pressure in fluidic cannels is a function of injection rate and channel dimensions. In one embodiment, the device provides an oil/surfactant inlet; an inlet for an analyte; a filter, an inlet for mRNA capture microbeads and lysis reagent; a carrier fluid channel which connects the inlets; a resistor; a constriction for droplet pinch-off, a mixer; and an outlet for drops. In an embodiment the invention provides apparatus for creating a single-cell sequencing library via a microfluidic system, which may comprise: an oil-surfactant inlet which may comprise a filter and a carrier fluid channel, wherein said carrier fluid channel may further comprise a resistor; an inlet for an analyte which may comprise a filter and a carrier fluid channel, wherein said carrier fluid channel may further comprise a resistor; an inlet for mRNA capture microbeads and lysis reagent which may comprise a filter and a carrier fluid channel, wherein said carrier fluid channel further may comprise a resistor; said carrier fluid channels have a carrier fluid flowing therein at an adjustable or predetermined flow rate; wherein each said carrier fluid channels merge at a junction; and said junction being connected to a mixer, which contains an outlet for drops. Accordingly, an apparatus for creating a single-cell sequencing library via a microfluidic system microfluidic flow scheme for single-cell RNA-seq is envisioned. Two channels, one carrying cell suspensions, and the other carrying uniquely barcoded mRNA capture bead, lysis buffer and library preparation reagents meet at a junction and is immediately co-encapsulated in an inert carrier oil, at the rate of one cell and one bead per drop. In each drop, using the bead's barcode tagged oligonucleotides as cDNA template, each mRNA is tagged with a unique, cell-specific identifier. The invention also encompasses use of a Drop-Seq library of a mixture of mouse and human cells. The carrier fluid may be caused to flow through the outlet channel so that the surfactant in the carrier fluid coats the channel walls. The fluorosurfactant can be prepared by reacting the perfluorinated polyether DuPont Krytox 157 FSL, FSM, or FSH with aqueous ammonium hydroxide in a volatile fluorinated solvent. The solvent and residual water and ammonia can be removed with a rotary evaporator. The surfactant can then be dissolved (e.g., 2.5 wt %) in a fluorinated oil (e.g., Fluorinert (3M)), which then serves as the carrier fluid. Activation of sample fluid reservoirs to produce regent droplets is based on the concept of dynamic reagent delivery (e.g., combinatorial barcoding) via an on-demand capability. The on-demand feature may be provided by one of a variety of technical capabilities for releasing delivery droplets to a primary droplet, as described herein.

From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional patent application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional patent application filed Sep. 21, 2012, and U.S. patent application Ser. No. 15/303,874 filed Apr. 17, 2015 entitled “Systems and Methods for Droplet Tagging.” Accordingly, in or as to the invention it is envisioned that there can be the dynamic generation of molecular barcodes (e.g., DNA oligonucleotides, fluorophores, etc.) either independent from or in concert with the controlled delivery of various compounds of interest (siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with CRISPR-Cas detection system-containing droplets. An electronic record in the form of a computer log file can be kept to associate the barcode delivered with the downstream reagent(s) delivered. This methodology makes it possible to efficiently screen a large population of samples according to the methods disclosed herein. The device and techniques of the disclosed invention facilitate efforts to perform studies that require data resolution at the single cell (or single molecule) level and in a cost-effective manner. A high-throughput and high-resolution delivery of reagents to individual emulsion droplets that may contain samples of target molecules for further evaluation through the use of monodisperse aqueous droplets that are generated one by one in a microfluidic chip as a water-in-oil emulsion.

### EXAMPLES

### Example 1—Bead-Based Approaches to CRISPR Diagnostics

Bead-Based Strategy to Couple Cas-13 Activity with a Split NanoLuciferase-Based Readout

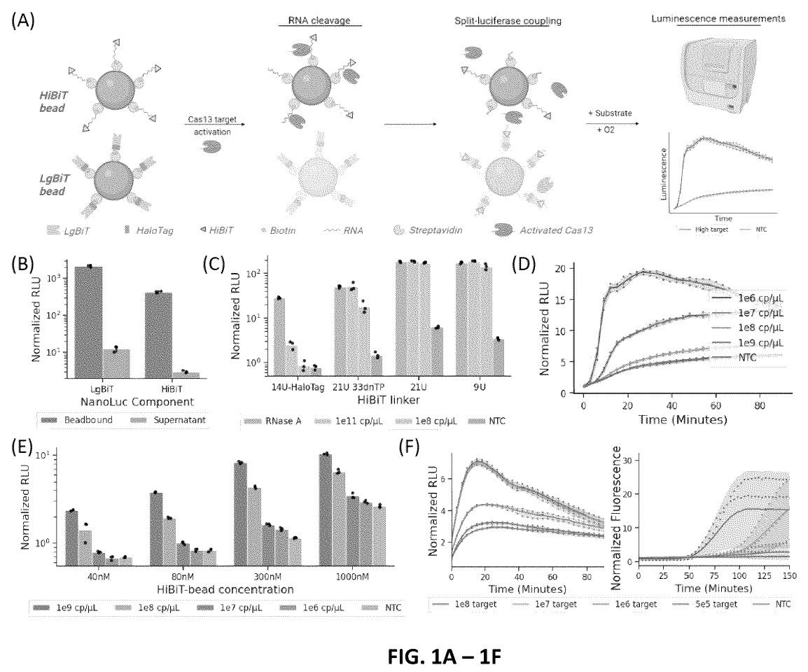

Applicant developed a bead-based Luciferase reporter (bbLuc) to couple Cas13 activity with a split Nanoluciferase-based readout (FIG. 1A). As a first step, Applicant probed whether it is possible to couple HiBiT and LgBIT to beads using a Cas13-cleavable RNA-based linker. Applicant attached HiBiT and LgBiT to biotinylated oligonucleotides using HaloLigand-HaloTag-based covalent linking, thereby enabling coupling to streptavidin coated beads (FIG. 1B) (Los et al. 2008). Notably, this coupling enabled target-mediated Cas13 cleavage of HiBiT-linked nanoparticles (FIG. 1C). However, Applicant did not observe significant Cas13 cleavage of LgBiT-linked nanoparticles, most likely due to increased steric hindrance of Cas13 cleavage in proximity with the larger LgBiT enzyme compared to the HiBiT peptide. As such, Applicant focused subsequent design efforts on cleavable HiBiT-nanoparticles.

Having demonstrated the ability to couple HiBiT to beads using a Cas13-cleavable RNA-based linker, Applicant next designed improved linkers to optimize and enable more efficient cleavage. Applicant found through a side-by-side comparison of Cas13-based and RNase A-based cleavage that the initial Cas13 cleavage of HaloTag-based linking was inefficient (FIG. 1C). Applicant hypothesized that this inefficiency may have been caused again by steric hindrance between the beads and HaloTag-HiBiT complex, reducing accessibility of Cas13 to cleavage sites. Applicant therefore increased linker length (Table 1) and changed linkage chemistry by using Strain-promoted Azide—Alkyne Click Chemistry reaction (SPAAC) to connect the HiBiT peptide to the oligonucleotide linker. This linkage enabled significantly more efficient Cas13 cleavage compared to the HaloTag-HiBiT, showing equivalent cleavage of the HiBiT linker from Cas13 and RNase A (FIG. 1C).

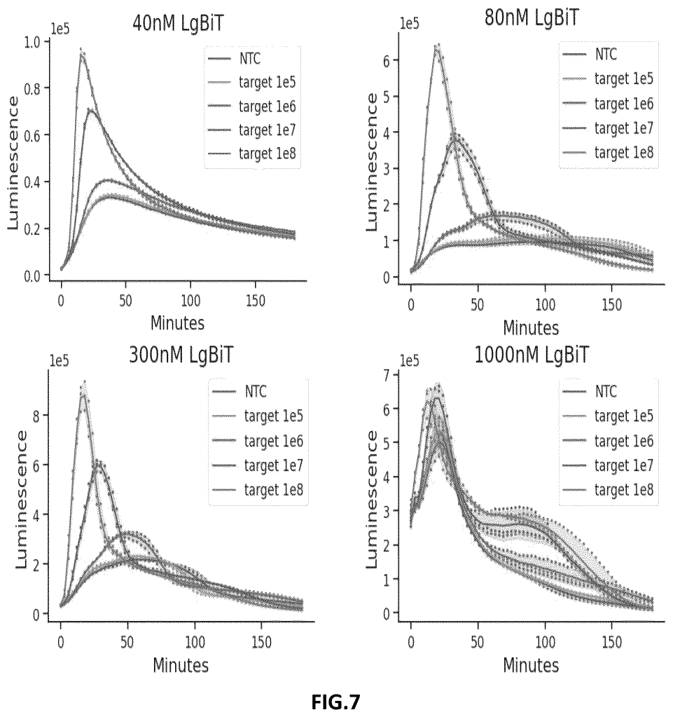

Next, Applicant characterized and further optimized the performance of an amplification-free assay with both HiBiT and LgBiT beads in solution. Using 80 nM HiBiT peptide coupled to nanoparticles in solution, Applicant was able to achieve detection down to 107 copies/uL of input RNA (FIG. 1D). While this compared favorably to a fluorescence-based amplification-free assay, Applicant observed further improvements in sensitivity when the surface density of HiBiT peptides on nanoparticles was increased to a concentration of 300 nM (FIG. 1E). HiBiT concentrations above 300 nM and LgBiT concentrations above 80 nM resulted in more inconsistency and lower sensitivity (FIG. 7), possibly due to an increased viscosity of the solution caused by a higher peptidic charge on the beads.

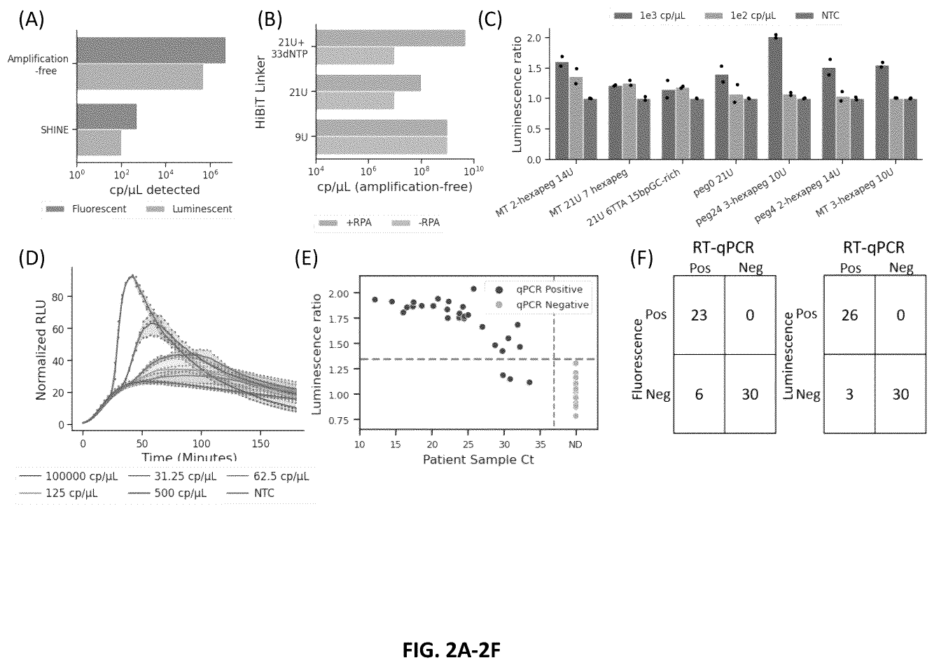

Applicant compared the performance of bbLuc to a conventional fluorescent reporter in the amplification-free assay. Applicant's luminescent reporter detected down to 5*105 copies/pL of input target compared to 5*106 copies/μL for the fluorescent reporter. Applicant was thus able to achieve a 10× increase in sensitivity using the luminescent reporter (FIGS. 1F, 2A).

Integration of Luminescent Reporter into SHINE

Having optimized bbLuc in an amplification-free setting, Applicant then assessed its performance in the diagnostic platform of SHINE. Applicant found that the luminescent reporter performed 5× better than the conventional fluorescent reporter in the SHINE setting, compared to the 10× enhancement Applicant achieved in the amplification-free setting (FIG. 2A). Applicant hypothesized that the greater enzymatic and buffer complexity of SHINE compared to amplification-free assays may have allowed for deleterious interactions between the bead complexes and reaction mixture, causing the observed 2× decrease in relative sensitivity.

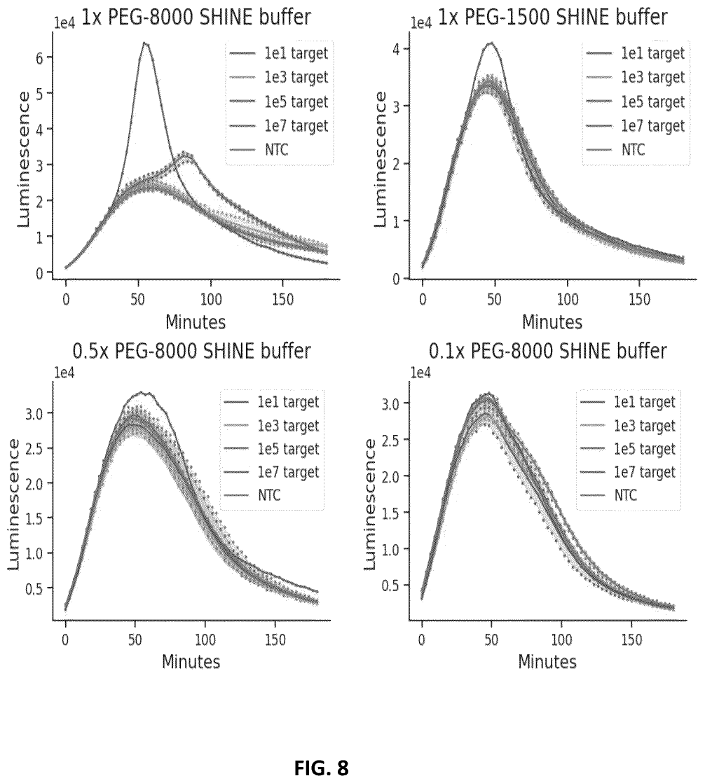

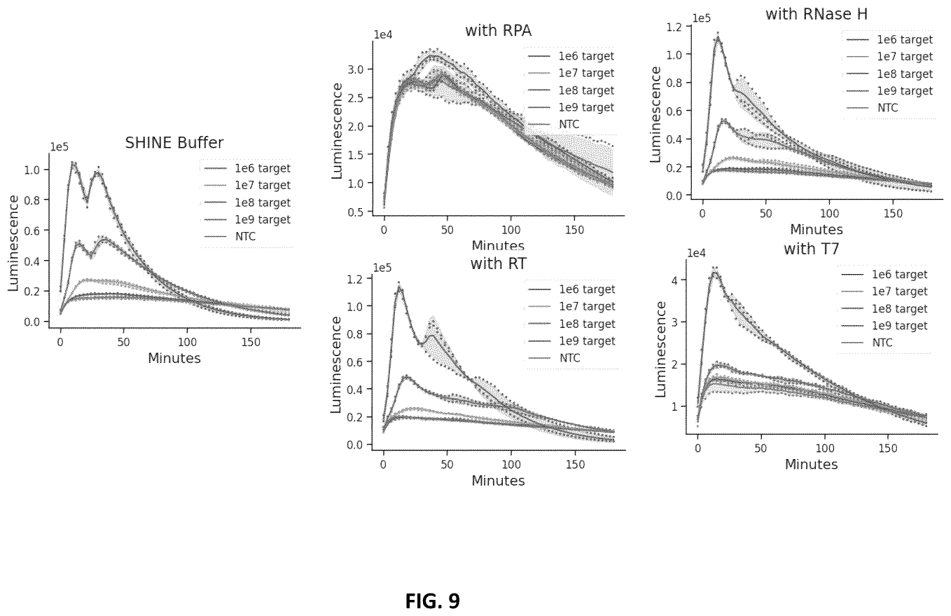

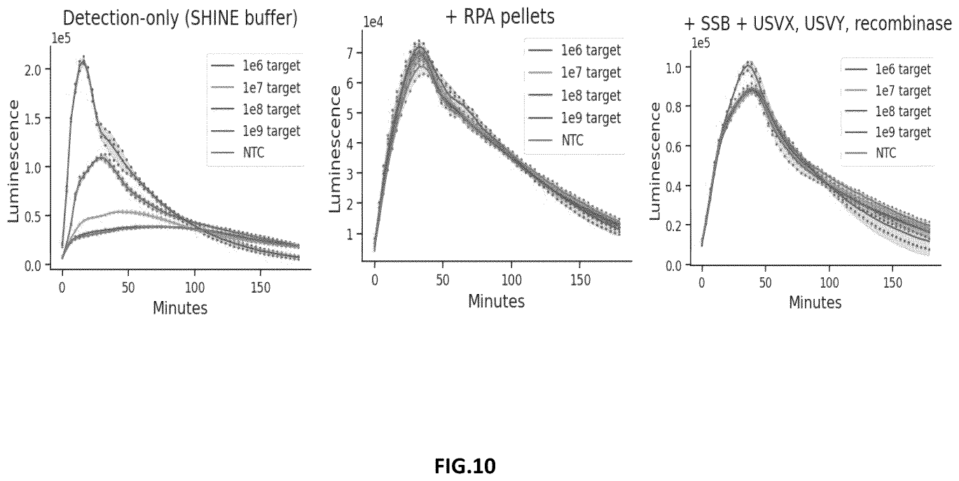

Applicant explored the potential causes of reduced sensitivity of Applicant's luminescent reporter in the SHINE context by testing different concentrations of a molecular crowding reagent and assessing if individually spiking in SHINE components to amplification-free reactions interfered with detection. Applicant first examined possible molecular crowding effects caused by bead addition to the assay by modifying polyethylene glycol (PEG) concentration and molecular weight in the SHINE reaction buffer, but found the PEG parameters in fluorescent SHINE were optimal with the luminescent system (FIG. 8). Applicant then probed possible inhibitory effects on the beads from the constituent SHINE proteins by individually spiking reaction components into separate amplification-free reactions, finding a 1000-fold reduction in performance upon addition of recombinase polymerase amplification (RPA) pellets (FIG. 9). Applicant found that this reduction in sensitivity was largely caused by single-stranded binding (SSB) protein reducing Cas13 cleavage of the beads (FIG. 10).

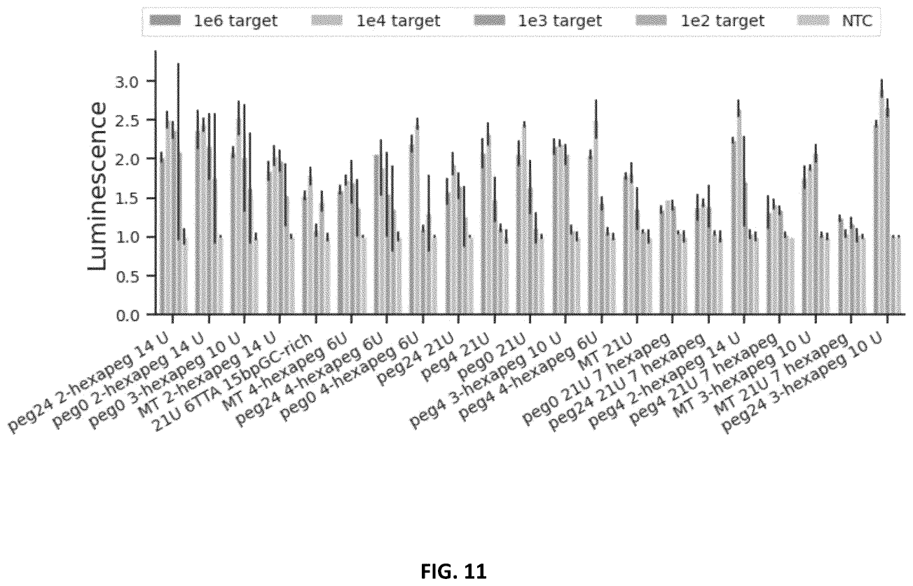

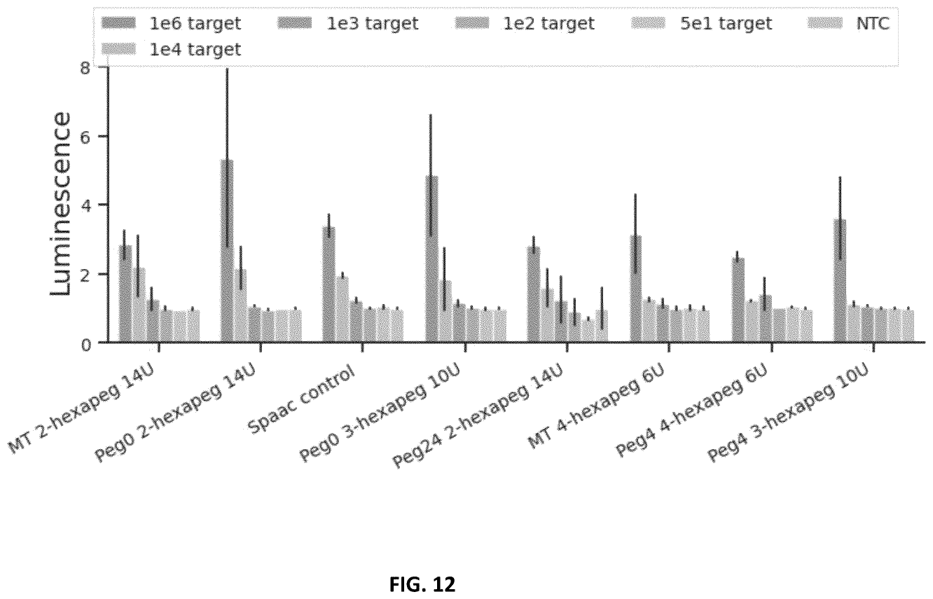

Based on the observation that the SSB used in RPA was inhibiting bead linkers, Applicant tested a series of new linkers to find designs that improved detection in SHINE. Applicant hypothesized that Applicant's original HiBiT-bead linker design, consisting of 54 nucleotides (21 uracils and 33 dNTPs) may have served as a binding site for SSB, thereby interfering with Cas13 cleavage of the linker. Applicant first tested shorter linker designs without any dNTPs, finding reduced inhibition of detection by 21U and 9U linkers in the presence of RPA components compared to the original design (FIG. 2B). Applicant went on to test 27 different linker designs (FIG. 2C, FIGS. 11-12, Table 1), finding that a 2-hexapeg 14U linker provided the highest sensitivity amongst reporters.

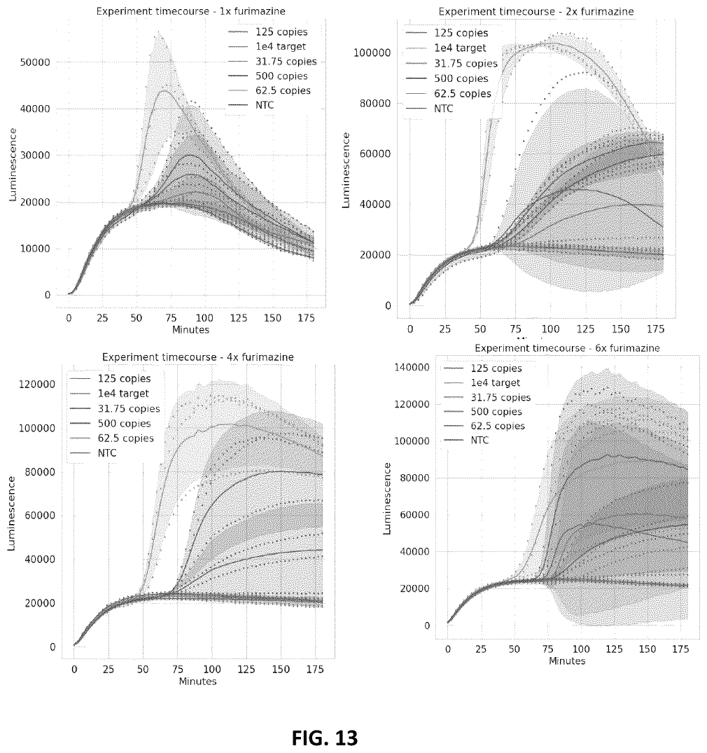

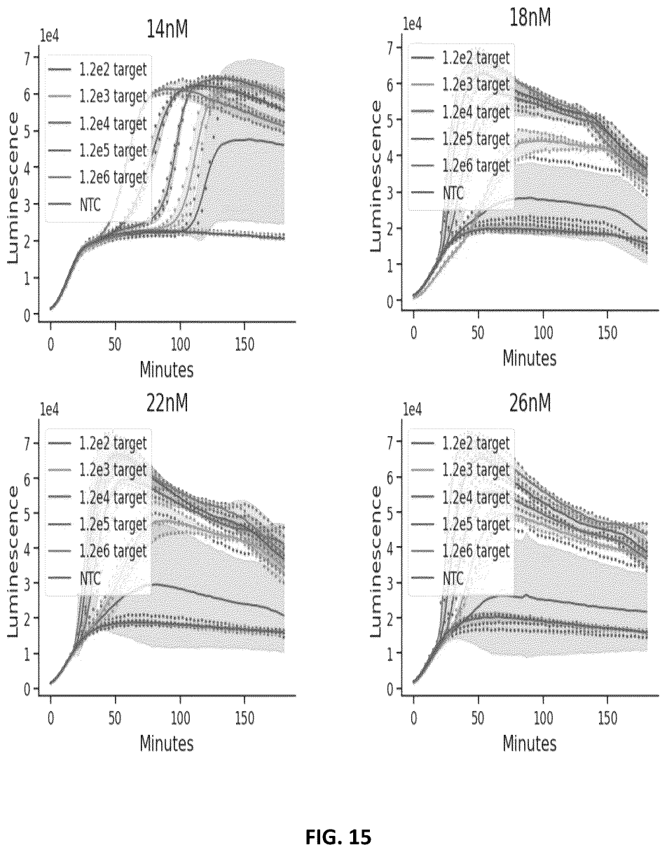

Applicant made a few final optimizations and measured the limit of detection (LOD) of the assay. Applicant varied the concentrations of furimazine, RPA primers, and magnesium acetate, determining optimal concentrations of 80 nM, 140 nM, and 14 nM, respectively (FIGS. 13-15). Applicant found an optimized assay had an LOD of 32 copies/uL of input RNA in 75 minutes (FIG. 2D), representing a 3× improvement of sensitivity following optimization.

### SARS-CoV-2 Clinical Sample Validation of Luminescent Reporters

Applicant compared the performance of bbLuc and fluorescent SHINE to a gold-standard RT-qPCR on RNA extracted from 63 clinical swabs from suspected COVID-19 patients. (FIG. 2E) Four out of 63 samples were negative for RNase P, a control gene that confirms adequate sample collection. Among the remaining 59 samples, 29 were RT-qPCR confirmed COVID-19 positive and 30 were confirmed COVID-19 negative. The remaining four were ruled inconclusive as they were negative for the Rnase P control test. bbLuc SHINE detected SARS-CoV-2 in 26 of the 29 positive samples (89.6% concurrence) compared to 23 of the 29 positive samples (79.3%) in fluorescent SHINE (FIG. 2F). Every positive sample detected by fluorescent SHINE was also detected as positive by Applicant's luminescent system, which additionally detected 3 high Ct (Ct>28.5) samples compared to the fluorescent SHINE. This demonstrates an increased sensitivity with luminescent reporters. Both fluorescent and luminescent SHINE correctly identified all 30 RT-qPCR negative samples as negative.

### Equipment-Free Bead-Based Droplet Generation for Multiplexed Fluorescent Cas13 Detection

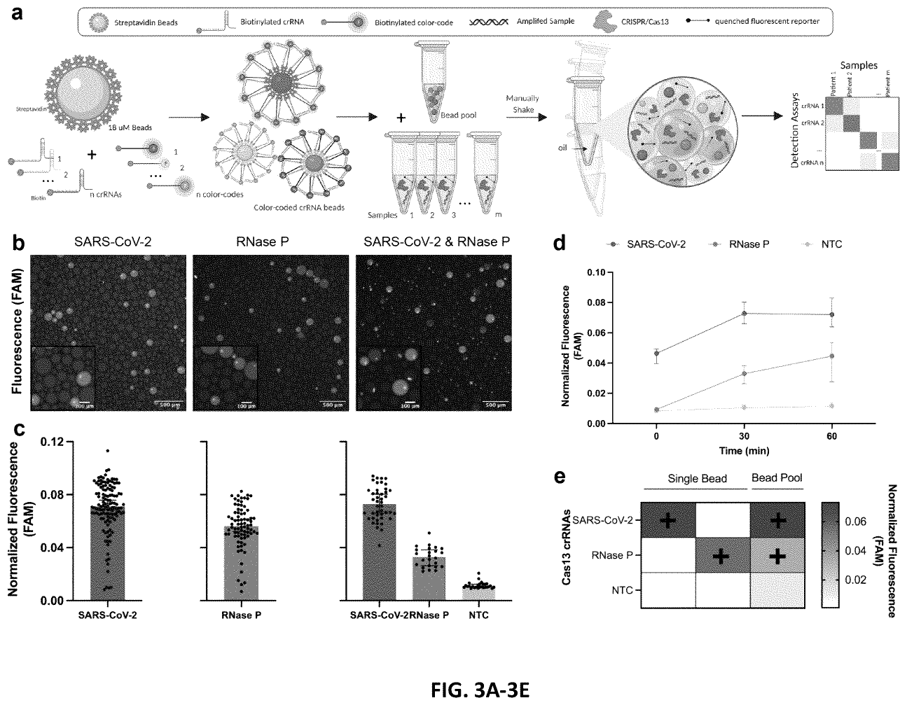

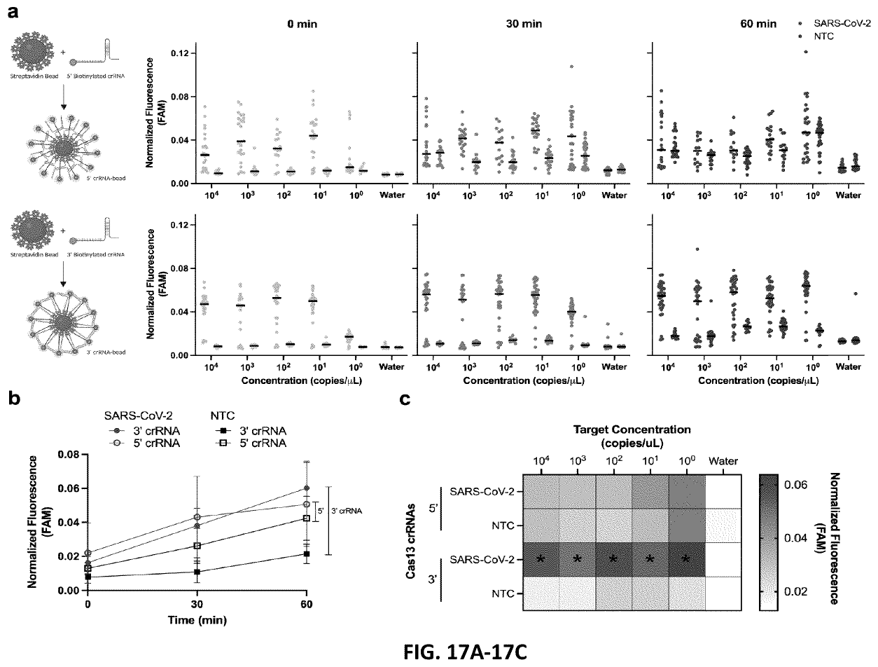

Next, Applicant developed a bead-based approach for localizing separation of reaction components to conduct discrete, multiplexed testing in the same overall solutions for point-of-care settings. To enable bead-based multiplexing of target detection, Applicant first attached target-specific biotinylated crRNAs to color-coded, streptavidin-coated beads, examining results when cRNA was attached to beads via either 3′ end biotinylation or via 5′ end biotinylation (FIG. 3A, FIG. 17A-17C). With 3′ end biotinylation, Applicant found significant Cas13 cleavage in target compared to no target control (NTC) down to 1 copy/uL of input. In comparison, with 5′ end biotinylation, Cas13 cleavage was not able to distinguish an input of 104 copies per microliter from NTC (FIG. 17D). As such, Applicant used 3′ biotinylated crRNA beads moving forward.

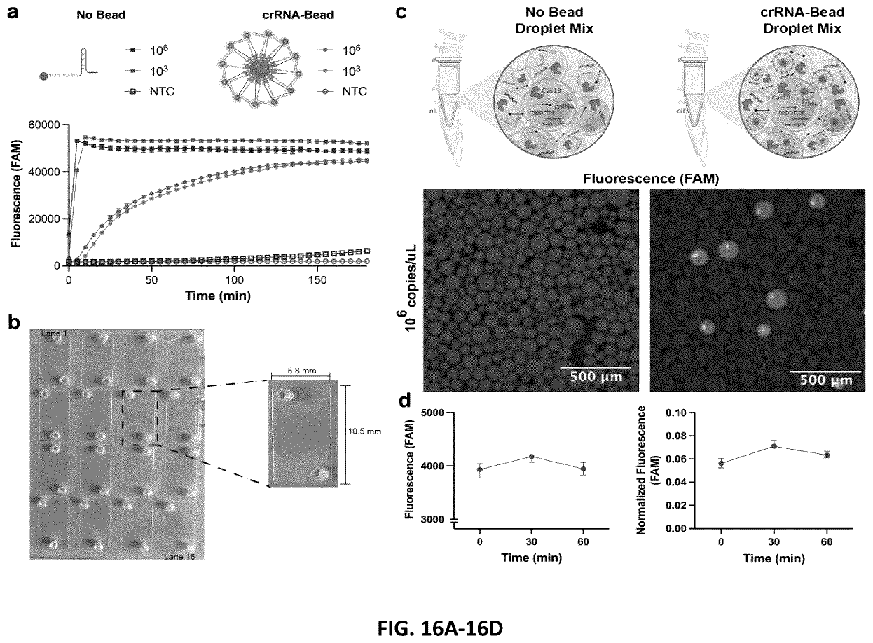

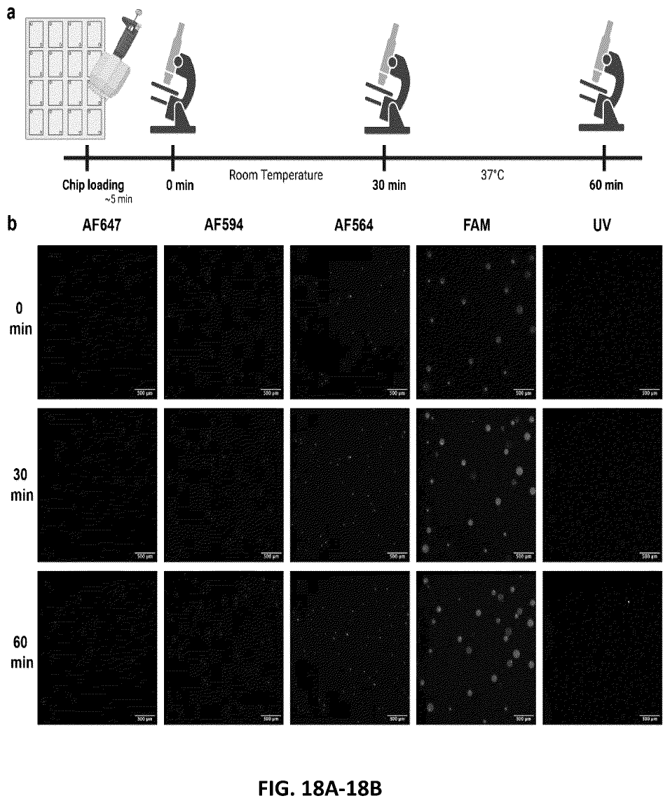

To show that Applicant's bead-based CARMEN (bbCARMEN) approach would work in the context of multiple targets, Applicant developed assays for SARS-CoV-2 and a human internal control RNAse P both individually and in combination (FIGS. 3B-3E, FIG. 18). Applicant added color-coded beads to a solution containing Cas13 and additional detection components with amplified patient samples. (FIG. 3A, FIG. 16A) For each reaction, Applicant combined the detection master mix with oil, shaking to form miniature droplets containing the master mix and one color-coded bead (FIG. 3A, FIG. 16C). Applicant then loaded samples on custom, prefabricated flow cells for readout using fluorescence microscopy and automated image analysis to track both the color-coded crRNA beads and the signal from Cas13 activity (FIG. 3B-D, FIG. 4A, FIG. 16B-16D). By 30 minutes, Applicant was successfully able to detect fluorescence intensity in droplets above background for all conditions (FIG. 3D-3E, FIG. 16C-16D, FIG. 18A-18B).

Respiratory Virus Panel Implementation on bbCARMEN

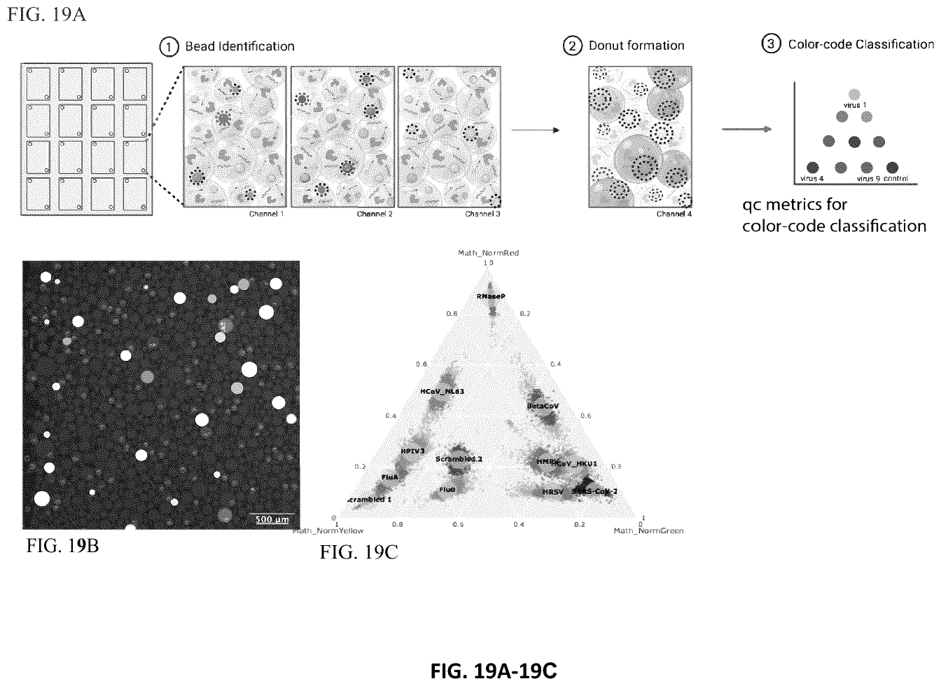

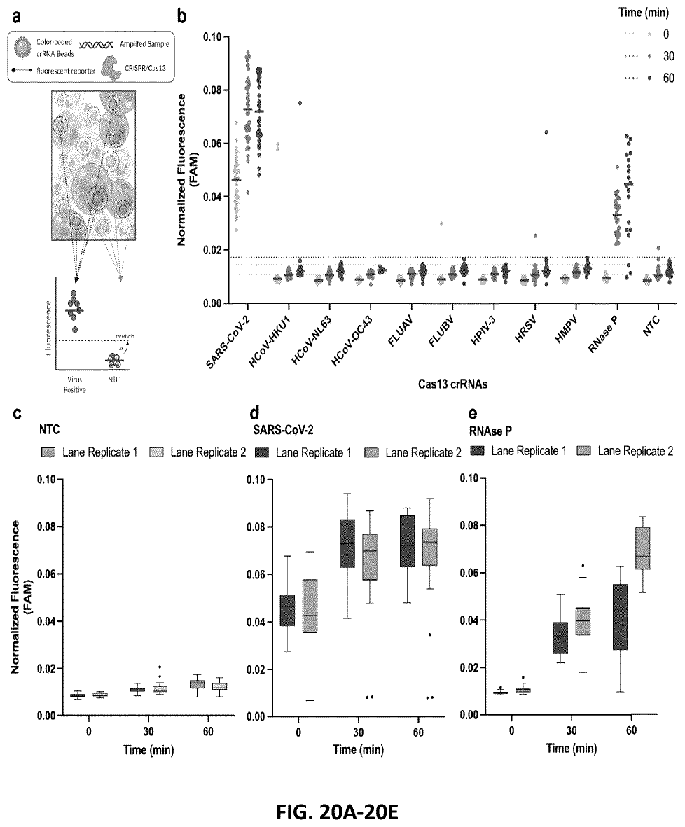

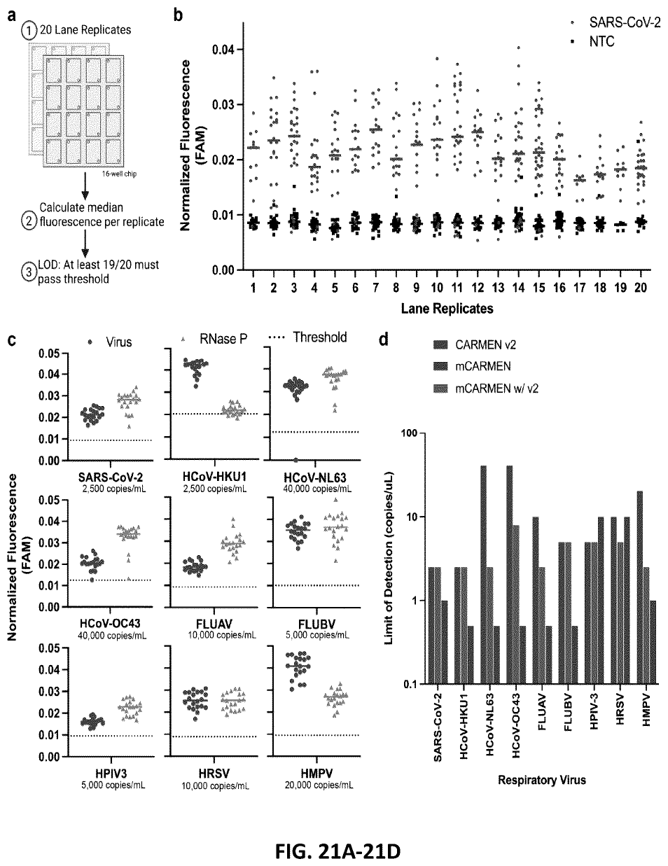

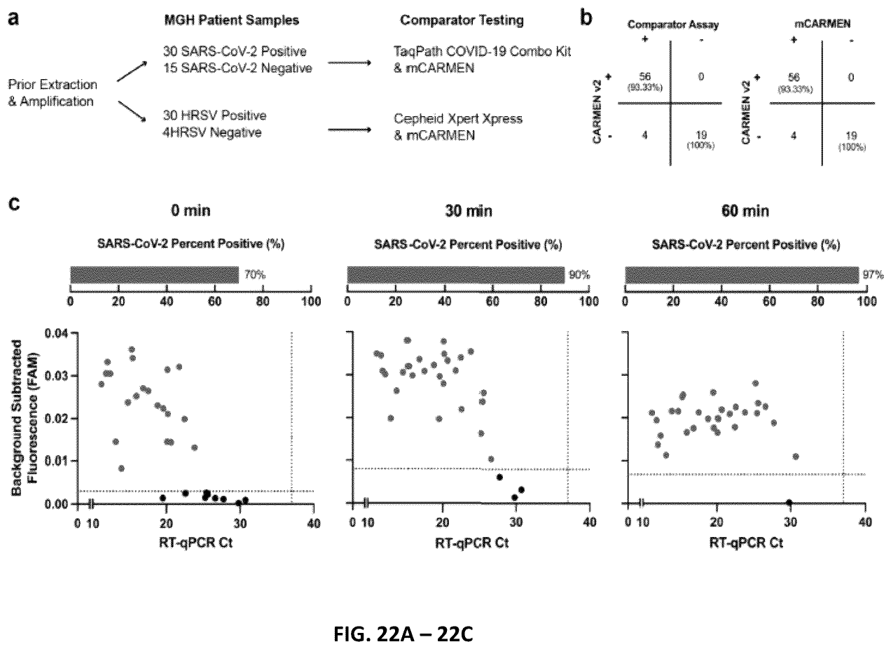

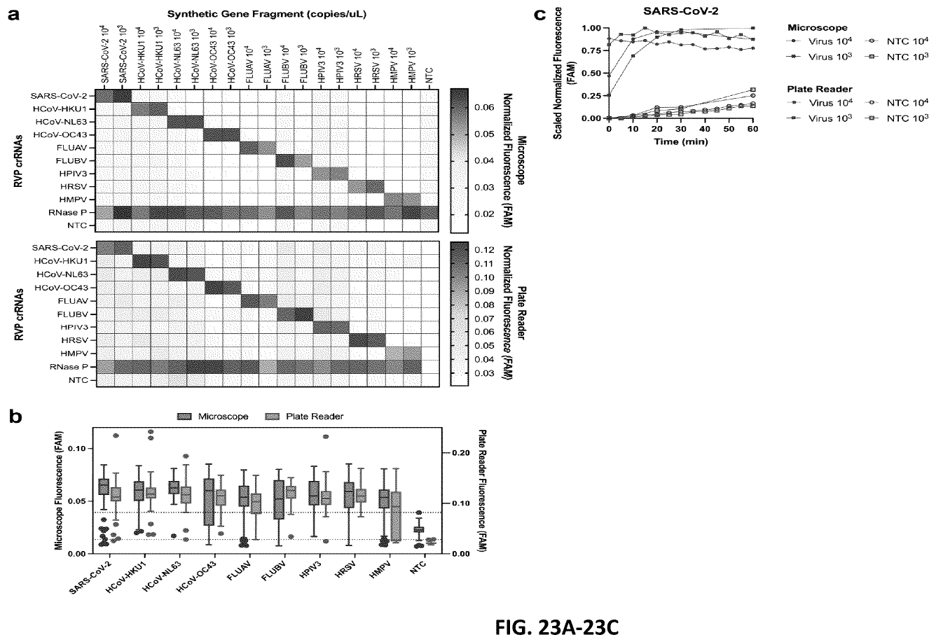

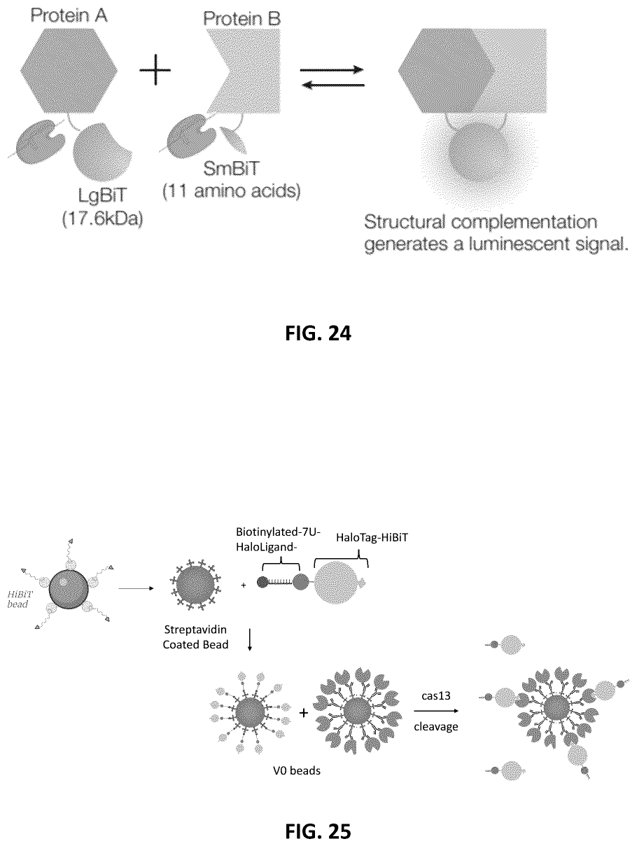

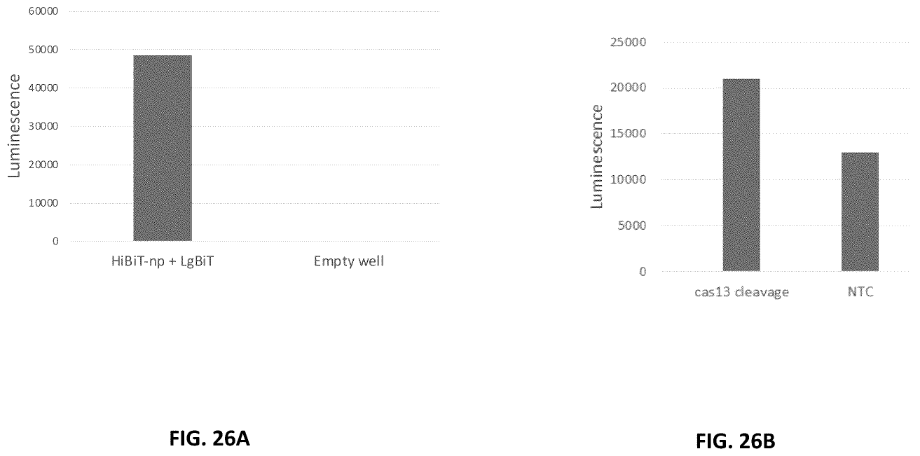

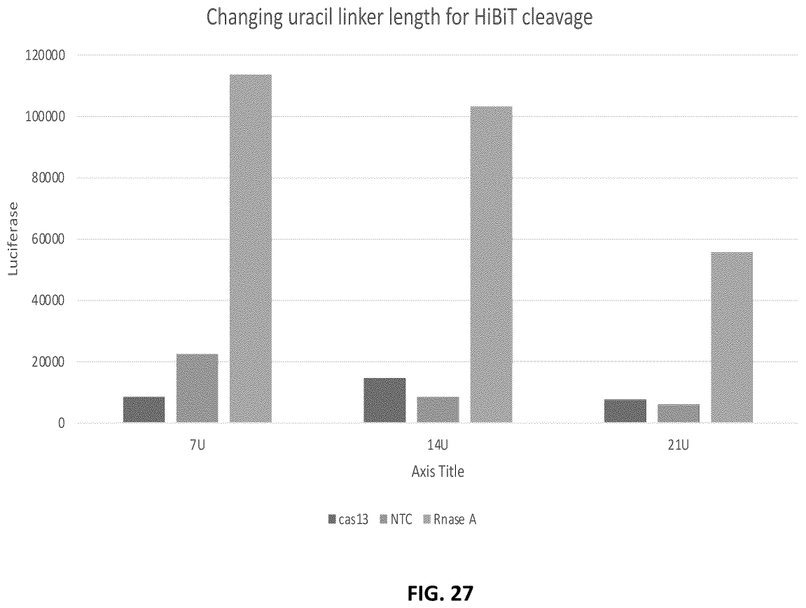

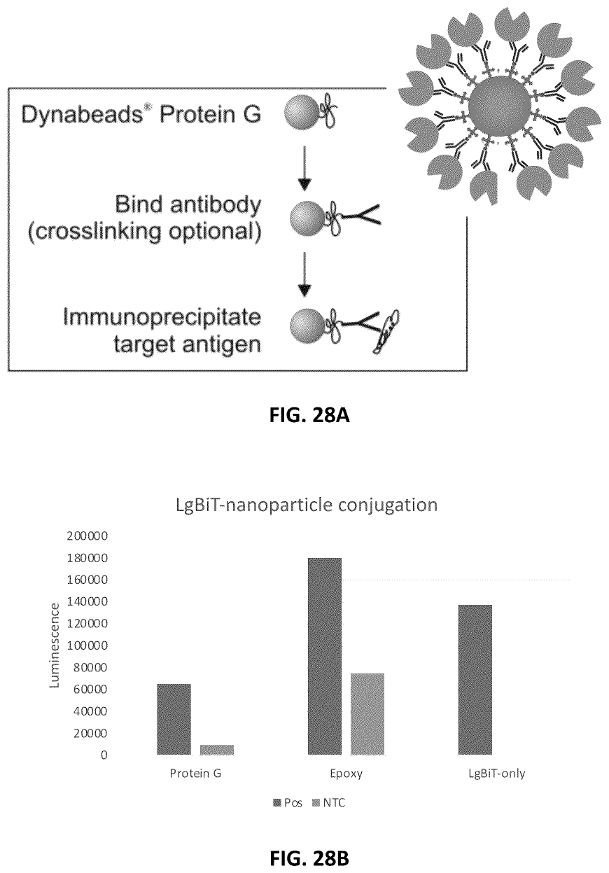

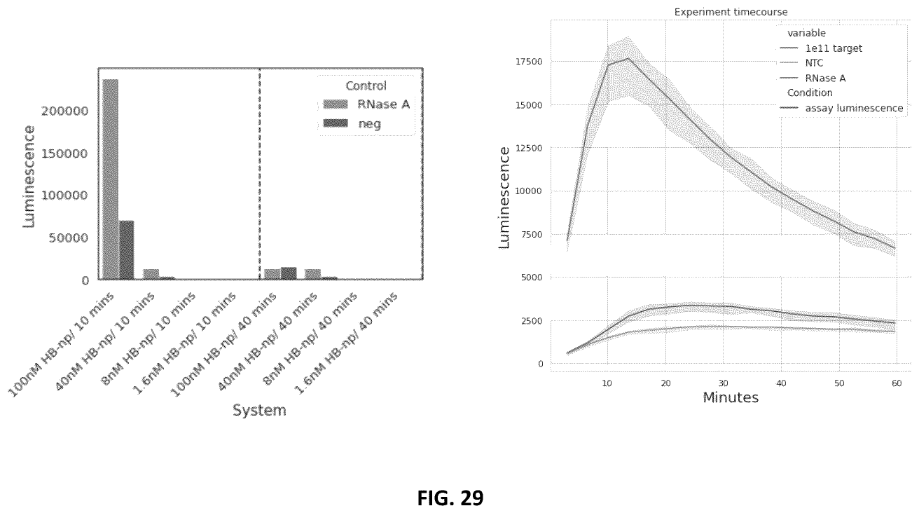

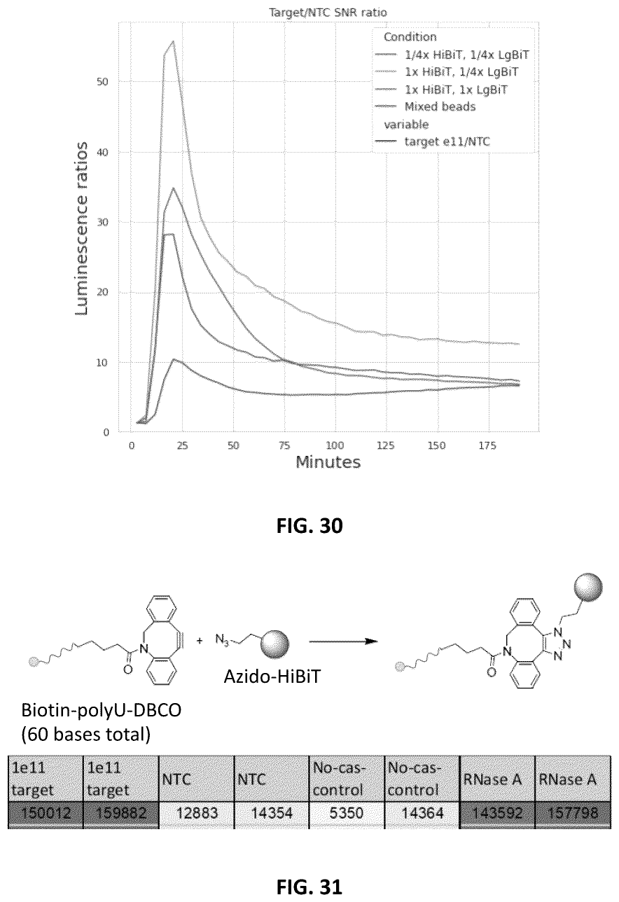

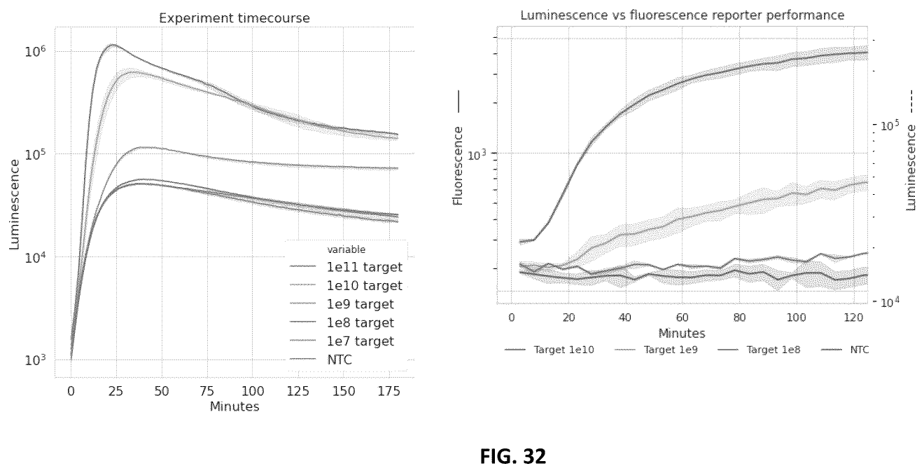

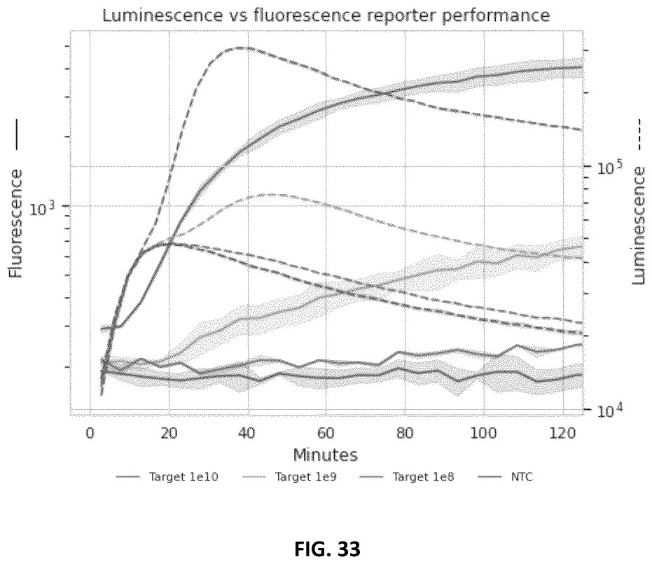

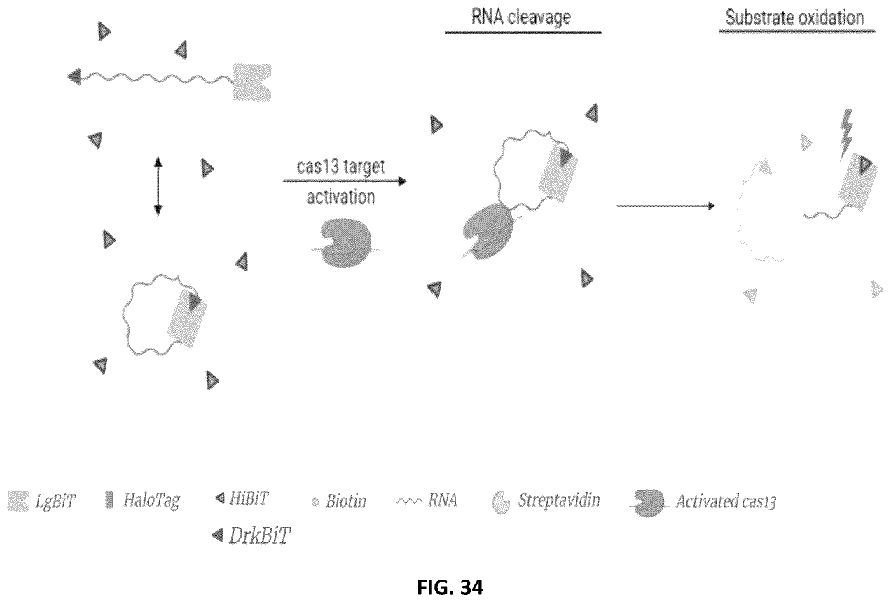

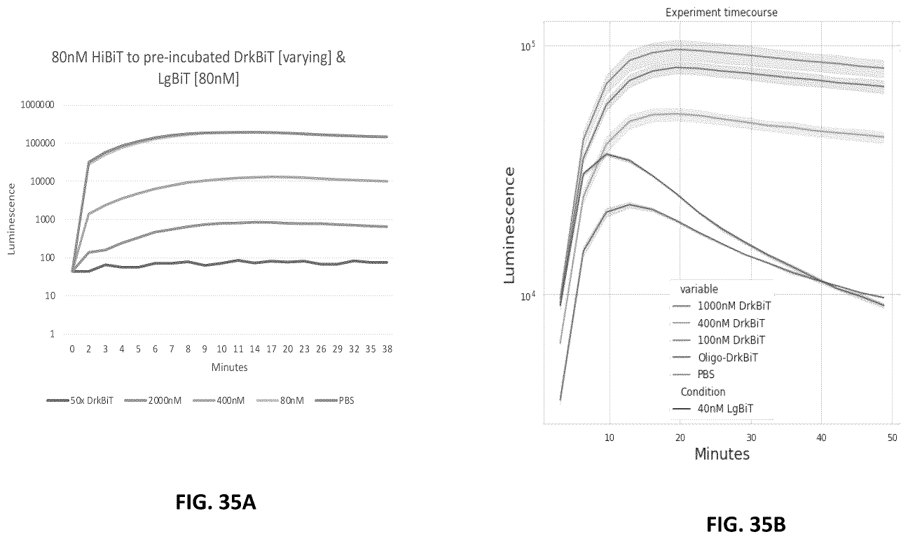

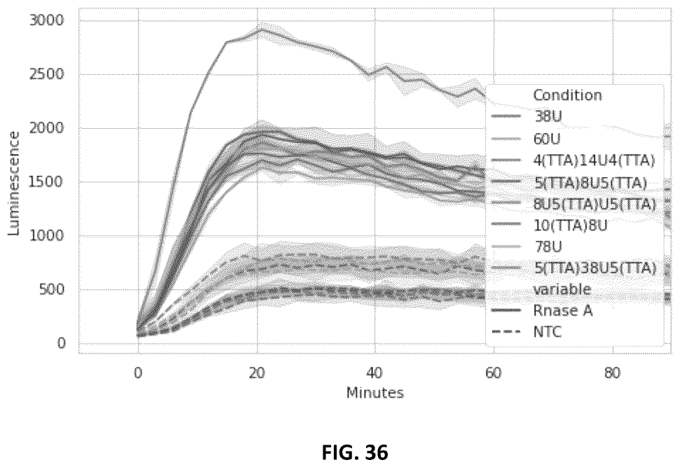

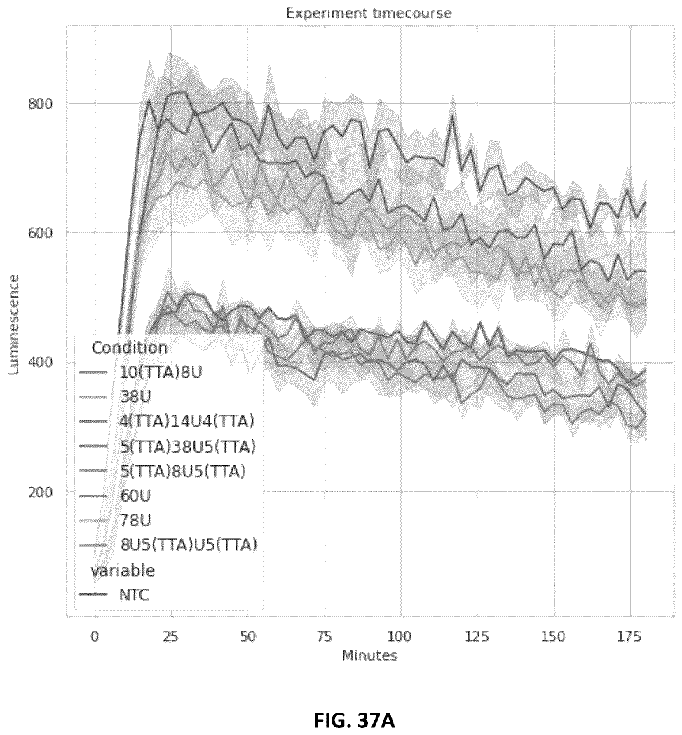

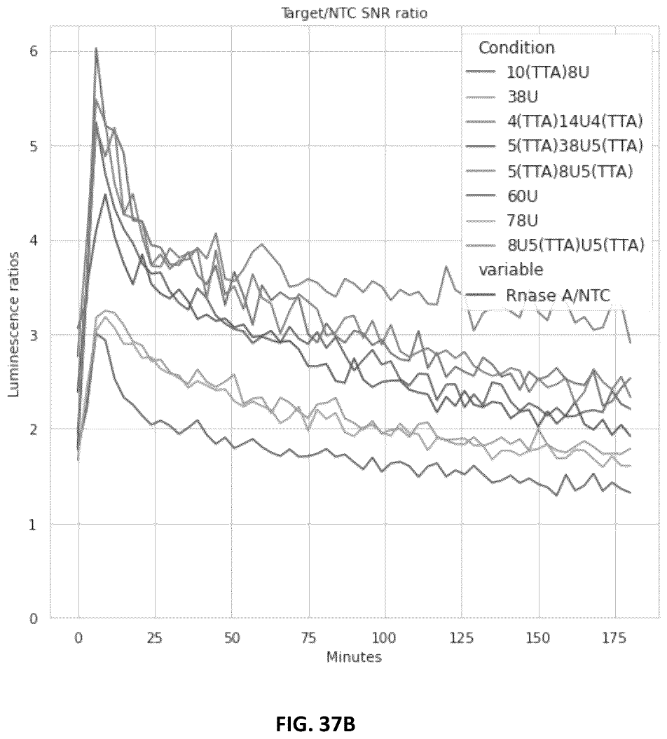

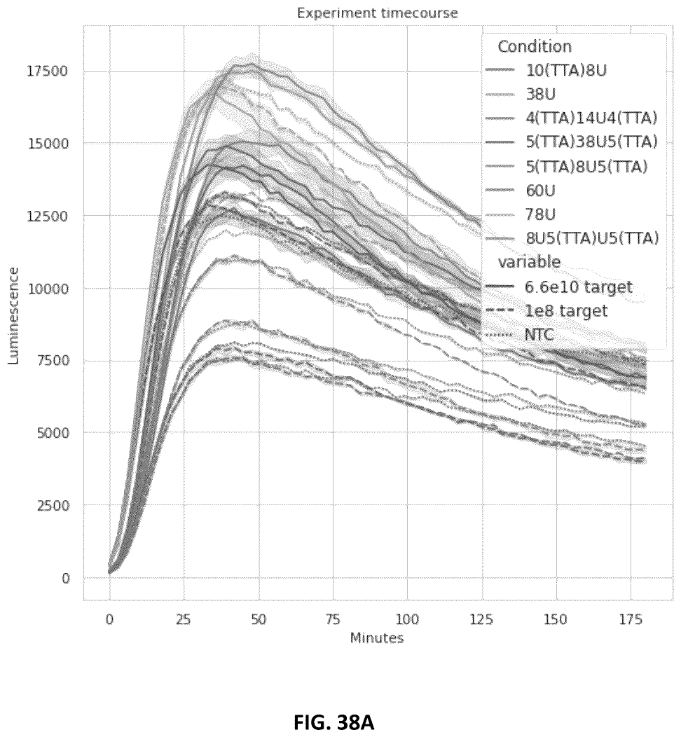

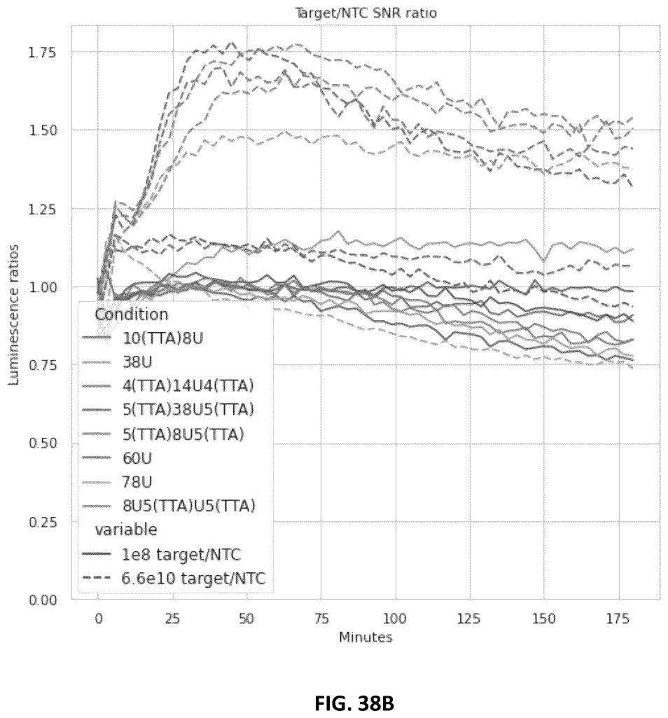

Applicant next tested the performance of a larger multiplexed bbCARMEN by implementing a 9-target Respiratory Virus Panel (RVP) previously characterized on the mCARMEN platform (FIG. 4A) (Welch et al. 2022). Applicant conjugated human RNase P crRNA (as an internal control) and each crRNA from the RVP to beads with target-specific color-codes and added the beads to the reaction along with patient sample (FIG. 19). Applicant used fluorescence microscopy to track patient sample signals over time and map crRNA/color-code combinations (FIG. 19).

With the RVP, bbCARMEN successfully distinguished all bead color-codes from one another across replicates, while simultaneously observing on-target signal with minimal-to-no off-target signal for each panel member (FIG. 4B-4C, FIG. 19). Applicant verified the accuracy of these results by conducting LOD studies for all panel members (FIG. 4B, FIGS. 20-21). Overall, Applicant found the LOD for each virus (SARS-CoV-2 and HCoV-HKU1: 2,500 copies/mL: FLUBV and HPIV3: 5,000 copies/mL: FLUAV and HRSV: 10,000 copies/mL, HMPV: 20,000 copies/mL; HCoV-NL63 and HCoV-OC43: 40,000 copies/mL) to be in line with or slightly higher than the LOD of the RVP on mCARMEN.

Applicant tested 47 freshly collected (Delta and Omicron) SARS-CoV-2 specimens and 9 negatives based on RT-qPCR and NGS results (FIG. 4D-4E) to more rigorously validate sensitivity. All but one positive specimen (97.9%) was deemed virus-positive by bbCARMEN within 60 minutes, and all negative samples were correctly deemed negative. To further assess the sensitivity of this assay, Applicant also tested bbCARMEN on two cohorts of virus-positive samples previously characterized with mCARMEN and clinically-validated comparator assays and stored in −80 C (FIG. 22). Of the 60 positive samples tested (30 SARS-CoV-2 positive and 30 HSRV positive), bbCARMEN detected 56 (93.33%) within 60 minutes, exhibiting robust sensitivity even in samples subjected to a freeze-thaw cycle.

Automated Readout of bbCARMEN Using Commercially Available Consumables and Equipment

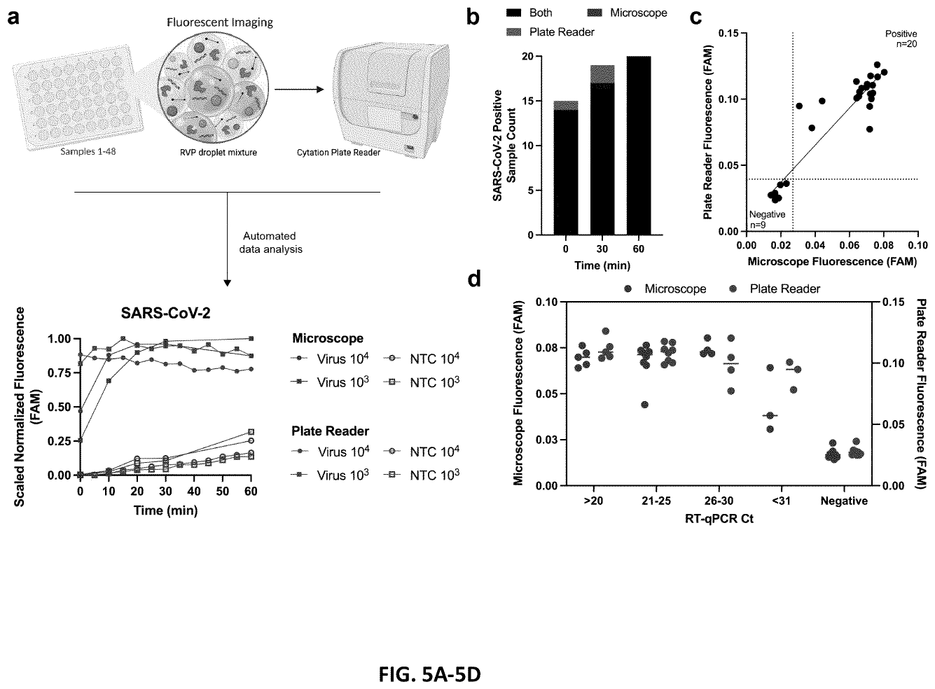

To further simplify deployment of this assay in low-resource settings, Applicant reconfigured bead loading and imaging steps to use a standard well plate and a lab plate reader instead of Applicant's previous custom flow cells and fluorescent microscope setup (FIG. 5A). Applicant found that 96-well plates loaded with a thin layer of droplets contained too many overlapping droplets of each color code for statistical significance in the final diagnostic calls. However, loading onto 48 well plates enabled the droplets to be spread out over a larger area, enabling us to distinguish between different color codes.

Applicant compared concordance of assay results between the microscopy-based and plate reader and found 100% concordance between the two readout platforms across synthetic material and patient specimens (FIG. 5B-5D, FIG. 23). Moreover, the plate reader method showed earlier discrimination between target conditions and NTC than Applicant's previous microscopy approach, suggesting an improvement in both assay workflow and performance (FIG. 23). As such, the changes to bead loading and imaging reduce equipment requirements, labor intensity, and data analysis expertise while maintaining sensitivity and sensitivity of bbCARMEN.

### Discussion

In this study, Applicant employed a novel bead-based approach to achieve point-of-need and point-of-care uses in CRISPR diagnostics. These technologies increase sensitivity and deployability in resource-constrained point-of-care settings. Applicant's luminescent bead-based approach, bbLuc, provides an attractive alternative to traditional fluorescence-based diagnostics, showing increased sensitivity in synthetic and clinical specimens. Critically, enhanced sensitivity also has upstream effects on assay adaptation to new or emerging pathogens by reducing the optimization time required to meet a target LOD. Furthermore, a luminescent assay reduces equipment requirements in conventional assays by removing the need for a light source for fluorescence excitation. These luminescence assays may be conducted with a luminescence detector, a smartphone camera, or even without any equipment (aside from a dark box providing darkness) as the assay can be read out by eye.

By utilizing a multiplexed bead-based system for point-of-care diagnostics, bbCARMEN addresses the significant equipment and expertise requirements of other multiplexed systems (Welch et al. 2022; Ackerman et al. 2020). bbCARMEN maintains excellent multiplexing ability and sensitivity by using beads as an operationally simple, inexpensive modality to perform multiplexed reactions with high specificity as shown in clinical sample testing. Implementation of Applicant's viral respiratory panel assay further demonstrates the ease of adaptability and its potential to dramatically increase deployability in resource-constrained settings.

There are numerous avenues to enhance this bead-based technology. For example, in bbLuc, Applicant focused on the cleavage of HiBiT-nanoparticles instead of LgBiT-nanoparticles. Due to manufacturing constraints, Applicant was not able to consistently manufacture an RNA linker long enough for Cas13 cleavage of LgBiT nanoparticles, but future work may incorporate new technologies in RNA synthesis or protein-oligo conjugation. In bbCARMEN, Applicant was able to successfully resolve 9 different crRNA color-codes, giving resolution to discriminate against different viral infections in a point-of-care setting. However, future work can be undertaken to improve the number of simultaneously assayable viruses by iterating on color code technology, either through the use of new fluorescent dyes, a different combinatorial barcoding strategy, or novel multicolor bead approaches.

The fundamental advance of these bead-based platforms may be applied in the future to other CRISPR-Cas based diagnostic platforms. Together, these technologies represent new modalities to increase diagnostic sensitivity and portability, opening avenues for the rapid and sensitive detection of biological molecules.

### Methods

### Clinical Samples

Human specimens from patients with SARS-CoV-2, HCoV-HKU1, HCoV-NL63, FLUAV, FLUBV, HRSV and HMPV were obtained from the Mass General Brigham IRB protocol no. 2019P003305.

### Sample Collection and Extraction

Patient samples were collected and stored in universal transport medium (UTM) or viral transport medium (VTM) and stored at −80 C. Samples for luciferase reporter testing were extracted using automatic nucleic acid extraction on the KingFisher Flex Magnetic Particle Processor with 96 Deep Well Head (Thermo Fisher Scientific) using MagMAX™ mirVana™ Total RNA Isolation Kit. For bbLuc, RNA was extracted from 100 μL of input volume and eluted into a final volume of 16 uL water and stored at −80 C.

### Oligonucleotides

The oligonucleotides used in these studies is shown below in Table 1.

TABLE 1

| Oligo name | Sequence |
|---|---|
| HaloLigand-based linker, | /5Biosg/rUrUrU rUrUrU rU/HaloLigand/ |
| 7U | |
| HaloLigand-based linker, | /5Biosg/rUrUrU rUrUrU rUrUrU rUrUrU rUrU/HaloLigand/ |
| 14U | (SEQ ID NO: 1) |
| HaloLigand-based linker, | /5Biosg/rUrUrU rUrUrU rUrUrU rUrUrU rUrUrU rUrUrU |
| 21U | rUrUrU/HaloLigand/ (SEQ ID NO: 2) |
| SPAAC linker, 21U, | /5Biosg/rUrUrU rUrUrU rUrUrU rUrUrU rUrUrU rUrUrU rUrUrU |
| 33 dNTPs | TTA TTA TTA TTA TTA TTA GGA GGA GCA CGA GGA/3DBCO/ |
| | (SEQ ID NO: 3) |
| Thiol 21U 7-hexapeg | /5Biosg/rUrU rUrUrU rUrUrU rUrUrU rUrUrU rUrUrU rUrUrU |
| | rU/iSp18//iSp18//iSp18//iSp18//iSp18//iSp18//iSp18// |
| | 3ThioMC3-D/ |
| | (SEQ ID NO: 4) |
| Thiol 2-hexapeg 14 U | /5Biosg//iSp18//iSp18//rUrUrU rUrUrU rUrUrU rUrUrU |
| | rUrU/3ThioMC3-D/ (SEQ ID NO: 5) |
| Thiol 3-hexapeg 10 U | /5Biosg//iSp18//iSp18//iSp18//rUrUrU rUrUrU rUrUrU rU/ |
| | 3ThioMC3-D/ (SEQ ID NO: 6) |
| Thiol 4-hexapeg 6U | /5Biosg//iSp18//iSp18//iSp18//iSp18//rUrUrUrUrUrU/ |
| | 3ThioMC3-D/ |
| SPAAC 2-hexapeg 14 U | /5Biosg//iSp18//iSp18/rUrUrU rUrUrU rUrUrU rUrUrU |
| | rUrU/3DBCON/ (SEQ ID NO: 7) |
| SPAAC 3-hexapeg 10 U | /5Biosg//iSp18//iSp18//iSp18/rUrUrU rUrUrU rUrUrU rU/ |
| | 3DBCON/ (SEQ ID NO: 8) |
| SPAAC 4-hexapeg 6U | /5Biosg//iSp18//iSp18//iSp18//iSp18/rUrUrU rUrUrU/3DBCON/ |
| SPAAC 4-hexapeg 14 U | /5Biosg//iSp18//iSp18//iSp18//iSp18/rUrUrU rUrUrU rUrUrU |
| | rUrUrU rUrU/3DBCON/ (SEQ ID NO: 9) |
| Thiol 2-hexapeg 21 U | /5Biosg//iSp18//iSp18//rUrUrU rUrUrU rUrUrU rUrUrU rUrUrU |
| | rUrUrU rUrUrU/3ThioMC3-D/ (SEQ ID NO: 10) |
| Thiol 4-hexapeg 21 U | /5Biosg//iSp18//iSp18//iSp18//iSp18//rUrUrU rUrUrU rUrUrU |
| | rUrUrU rUrUrU rUrUrU rUrUrU/3ThioMC3-D/ (SEQ ID NO: 11) |
| Spike 69/70 gBlock | gaaatTAATACGACTCACTATAggTGACAAAGTTT |
| | TCAGATCCTCAGTTTTACATTCAACTCAGGACTTG |
| | TTCTTACCTTTCTTTTCCAATGTTACTTGGTTCCA |
| | TGCTATACATGTCTCTGGGACCAATGGTACTAAGA |
| | GGTTTGATAACCCTGTCCTACCATTTAATGATGGT |
| | GTTTATTTTGCTTCCACTGAGAAGTCTAACATAAT |
| | AAGAGGCTGGATTTTTGGTACTACTTTAGATTCGA |
| | AGACCCAGTCCCTACTTATTGTTAATAACGCTACT |
| | AATGTTGTTATTAAAGTCTGTGAATTTC |
| | (SEQ ID NO: 12) |
| Spike RPA primer | gaaatTAATACGACTCACTATAgggCAACTCAGGA |
| forward | CTTGTTCTTACCTTTCTTTTCC (SEQ ID NO: 13) |
| Spike RPA primer reverse | AAGCAAAATAAACACCATCATTAAAT (SEQ ID NO: 14) |
| Spike crRNA 69/70 | rGrArU rUrUrA rGrArC rUrArC rCrCrC rArArA rArArC rGrArA |
| | rGrGrG rGrArC rUrArA rArArC rArCrA rGrGrG rUrUrA rUrCrA |
| | rArArC rCrUrC rUrUrA rGrUrA rCrCrA rU (SEQ ID NO: 15) |
| FAM_7U_Reprter | /56-FAM/rUrUrUrUrUrUrU/3IABkFQ/ |
| SARS-CoV-2 crRNA | GAUUUAGACUACCCCAAAAACGAAGGGGACUAAAACCUAAA |
| | ACUAUUCACUUCAAUAGUCUGAA/3Bio/ (SEQ ID NO: 16) |
| HCoV-HKU1 crRNA | GAUUUAGACUACCCCAAAAACGAAGGGGACUAAAACAAUAU |
| | GAUUACCAUUACCACAAAAAUUA/3Bio/ (SEQ ID NO: 17) |
| HCoV-NL63 crRNA | GAUUUAGACUACCCCAAAAACGAAGGGGACUAAAACUUAAU |
| | AGUUUCAGCCGCAAAGAGUCUAA/3Bio/ (SEQ ID NO: 18) |
| HCoV-OC43 (BetaCoV) | GAUUUAGACUACCCCAAAAACGAAGGGGACUAAAACUGUUG |
| crRNA | UAACGCCCUUAUAAUAGACCUUA/3Bio/ (SEQ ID NO: 19) |
| HPIV3 crRNA | GAUUUAGACUACCCCAAAAACGAAGGGGACUAAAACGUCGC |
| | AUUUUCCCCUCAAUAGAGUCCUU/3Bio/ (SEQ ID NO: 20) |
| FluA crRNA | GAUUUAGACUACCCCAAAAACGAAGGGGACUAAAACAAAAA |
| | GCUUGUGAAUUCAAAUGUCCCUG/3Bio/ (SEQ ID NO: 21) |
| FluB crRNA | GAUUUAGACUACCCCAAAAACGAAGGGGACUAAAACACUAA |
| | ACAGAUCAGGACAAGGUAUUUGG/3Bio/ (SEQ ID NO: 22) |
| HMPV crRNA | GAUUUAGACUACCCCAAAAACGAAGGGGACUAAAACGUCGC |
| | AAAAGACAUGGUCUCCUCUUGUU/3Bio/ (SEQ ID NO: 23) |
| HRSV crRNA | GAUUUAGACUACCCCAAAAACGAAGGGGACUAAAACGUCUU |
| | UUUCUAGGACAUUGUAUUGAACA/3Bio/ (SEQ ID NO: 24) |
| RNaseP crRNA | GAUUUAGACUACCCCAAAAACGAAGGGGACUAAAACUCCGA |
| | GUCAGUGGCUCCCGUGUGUCGGU/3Bio/ (SEQ ID NO: 25) |
| Scrambled 1 crRNA | GAUUUAGACUACCCCAAAAACGAAGGGGACUAAAACACGUC |
| | UAAUACGAUACAUCAUUACAUAU/3Bio/ (SEQ ID NO: 26) |
| Scrambled 2 crRNA | GAUUUAGACUACCCCAAAAACGAAGGGGACUAAAACGUGCG |
| | CCGUUGGCUCGUGUAGCAGUUCC/3Bio/ (SEQ ID NO: 27) |
| SARS-CoV-2 forward primer | gaaatTAATACGACTCACTATAgggCAATTAGAGATGGAACTTAC |
| | ACC (SEQ ID NO: 28) |
| HCoV-HKU1 forward primer | gaaatTAATACGACTCACTATAgggGTGTGTTAAAAGTCAATCTCC |
| | TCG (SEQ ID NO: 29) |
| HCoV-NL63 forward primer | gaaatTAATACGACTCACTATAgggACTTGCTAATGATGTTAAAG |
| | ATACAC (SEQ ID NO: 30) |
| HCoV-OC43 (BetaCoV) | gaaatTAATACGACTCACTATAgggGCTAAGAATGAGAGTAGTTC |
| forward primer | ATTG (SEQ ID NO: 31) |
| HPIV3 forward primer | gaaatTAATACGACTCACTATAgggTGATCTCAATGAAATTAGAA |
| | AGATGG (SEQ ID NO: 32) |
| FluA forward primer | gaaatTAATACGACTCACTATAgggGAGCAAAAAGAAGTCCTATA |
| | TAAATAAG (SEQ ID NO: 33) |
| FluB forward primer | gaaatTAATACGACTCACTATAgggCAAGCAAAACAAAAAGACTA |
| | AAGGC (SEQ ID NO: 34) |
| HMPV forward primer | gaaatTAATACGACTCACTATAgggACCCAAATGAGAAAGACTGT |
| | G (SEQ ID NO: 35) |
| HRSV forward primer | gaaatTAATACGACTCACTATAgggCTTCACGAAGGCTCCACATA |
| | (SEQ ID NO: 36) |
| RNaseP forward primer | gaaatTAATACGACTCACTATAgggTTGATGAGCTGGAGCCA (SEQ |
| | ID NO: 37) |
| SARS-CoV-2 reverse primer | CTTTTTAGCTTCTTCCACAATGTC (SEQ ID NO: 38) |
| HCoV-HKU1 reverse primer | AACCATAAGGAGCATTTTGAAC (SEQ ID NO: 39) |
| HCoV-NL63 reverse primer | GACTTAACACTCTCTTCTTTAGCT (SEQ ID NO: 40) |
| HCoV-OC43 (BetaCoV) | ATTTACAGCACTAGAACTTTCATG (SEQ ID NO: 41) |
| reverse primer | |
| HPIV3 reverse primer | CTGATATCTCGCTTGGAACATCTGCAG (SEQ ID NO: 42) |
| FluA reverse primer | AATTAGCCACAAATCCATAGCG (SEQ ID NO: 43) |
| FluB reverse primer | TGTTTCTTCATTATATCTTTCTAATGGTAT (SEQ ID NO: 44) |
| HMPV reverse primer | GCAACATTAATTCCTGCTGCT (SEQ ID NO: 45) |
| HRSV reverse primer | CCCATATTGTTAGTGATGCAGG (SEQ ID NO:46) |
| RNaseP reverse primer | ATGTGGATGGCTGAGTTGTT (SEQ ID NO: 47) |
| SARS-CoV-2 gBlock | GCACCCATATTGTTAGTG (SEQ ID NO: 48) |
| HCoV-HKU1 gBlock | gaaatTAATACGACTCACTATAgggATGCTCTTCT |
| | TTCTATTCAGAATGGTTTTAGTGCTACCAACTCTG |
| | CACTTGCTAAAATACAAAGTGTTGTTAATTCTAAT |
| | GCTCAAGCACTTAATAGTTTGTTACAGCAATTATT |
| | TAATAAATTTGGTGCAATTAGTTCTTCTTTACAAG |
| | AAATTTTATCTCGTCTCGATGCTTTAGAGGCTCAG |
| | GTTCAGATTGATAGGCTTATTAATGGTCGTTTAAC |
| | TGCTTTAAATGCTTATGTCTCTCAACAGCTTAGTG |
| | ATATTTCTCTTGTAAAATTTGGTGCTGCTTTAGCT |
| | ATGGAGAAGGTTAATGAGTGTGTTAAAAGTCAATC |
| | TCCTCGTATTAATTTTTGTGGTAATGGTAATCATA |
| | TTTTGTCATTAGTTCAAAATGCTCCTTATGGTTTG |
| | TTGTTTATGCATTTTAGTTATAAACCTATTTCTTT |
| | TAAAACTGTTTTAGTAAGTCCTGGTTTGTGTATAT |
| | CAGGTGATGTAGGTATTGCACCTAAACAAGGGTAT |
| | (SEQ ID NO: 49) |
| HCoV-NL63 gBlock | gaaatTAATACGACTCACTATAgggACGTTATGTG |
| | TCTTTAGCTATTGATGCATACCCTCTTTCAAAACA |
| | CCCTAATTCTGAATATCGTAAGGTTTTTTACGTAT |
| | TACTTGATTGGGTTAAGCATCTTAACAAAAATTTG |
| | AATGAGGGTGTTCTTGAATCTTTTTCTGTTACACT |
| | TCTTGATAATCAAGAAGATAAGTTTTGGTGTGAAG |
| | ATTTTTATGCTAGTATGTATGAAAATTCTACAATA |
| | TTGCAAGCTGCTGGTTTATGTGTTGTTTGTGGTTC |
| | ACAAACTGTACTTCGTTGTGGTGATTGTCTGCGTA |
| | AGCCTATGTTGTGCACTAAATGCGCATATGATCAT |
| | GTATTTGGTACCGACCACAAGTTTATTTTGGCTAT |
| | AACACCGTATGTATGTAATGCATCAGGTTGTGGTG |
| | TTAGTGATGTCAAAAAATTGTATCTTGGTGGTTTG |
| | AATTACTATTGTACAAATCATAAACCACAGTTGTC |
| | TTTTCCATTATGTTCAGCTGGTAATATATTTGGTT |
| | TATATAAAAATTCAGCAACTGGTTCCTTAGATGTT |
| | GAAGTTTTTAATAGGCTTGCAACGTCTGATTGGAC |
| | TGATGTTAGGGACTATAAACTTGCTAATGATGTTA |
| | AAGATACACTTAGACTCTTTGCGGCTGAAACTATT |
| | AAAGCTAAAGAAGAGAGTGTTAAGTCTTCTTATGC |
| | TTTTG (SEQ ID NO: 50) |
| HCoV-OC43 (BetaCoV) | gaaatTAATACGACTCACTATAgggGTTGTAGATG |
| gBlock | AAGTTAGCATGCTTACCAATTATGAGCTTTCTGTT |
| | ATTAATGCTCGTATTCGTGCTAAGCATTATGTTTA |
| | TATTGGTGATCCTGCTCAATTGCCAGCACCACGTG |
| | TGTTATTGAGCAAGGGTACACTTGAACCTAAATAT |
| | TTTAACACTGTTACTAAGCTCATGTGTTGCTTAGG |
| | GCCAGACATTTTTCTTGGTACATGTTATAGATGTC |
| | CTAAGGAAATTGTTGATACAGTGTCCGCCTTGGTT |
| | TATGAAAATAAGCTTAAGGCTAAGAATGAGAGTAG |
| | TTCATTGTGTTTTAAGGTCTATTATAAGGGCGTTA |
| | CAACACATGAAAGTTCTAGTGCTGTAAATATGCAG |
| | CAGATTTATTTGATTAATAAGTTTTTGAAGGCTAA |
| | CCCTTTGTGGCATAAAGCTGTTTTTATTAGCCCAT |
| | ATAATAGTCAGAACTTTGCAGCTAAGCGTGTTTTG |
| | GGTTTACAAACCCAAACCGTGGATTCTGCTCAAGG |
| | (SEQ ID NO: 51) |
| HPIV3 gBlock | gaaatTAATACGACTCACTATAgggACCATCTGTC |
| | AACCAGAAATCAAACCAACAGAAACAAGTGAAAAA |
| | GATAGTGGATCAACTGACAAAAATAGACAGTCTGG |
| | GTCATCACACGAATGTACAACAGAAGCAAAAGATA |
| | GAAATATTGATCAGGAAACTGTACAGAGAGGACCT |
| | GGGAGAAGAGGCAGCTCAGATAGTAGAGCTGAGAC |
| | TGTGGTCTCTGGAGGAATCTCCAGAAGCATCACAG |
| | ATTCTAAAAATGGAACCCAAAACACGGAGAATATT |
| | GATCTCAATGAAATTAGAAAGATGGATAAGGACTC |
| | TATTGAGGGGAAAATGCGACAATCTGCAGATGTTC |
| | CAAGCGAGATATCAGGAAGTGATGGCATATTTACA |
| | ACAGAACAAAGTAGAAACAGTGATCATGGAAGAAG |
| | CTTGGAATCTATCGGTACACCTGATACAAGATCAA |
| | TAAGTGTTGTTACTGCTGCAACACCAGATGATGAA |
| | GAAGAAATACTAATGAGAAATAGTAGGATGAAGAA |
| | (SEQ ID NO: 52) |
| FluA gBlock | gaaatTAATACGACTCACTATAgggTGAATCAACA |
| | AGGAAGAAAATTGAGAAGATAAGGCCTCTTTTAAT |
| | GGATGGCACAGCATCACTGAGTCCTGGGATGATGA |
| | TGGGCATGTTCAACATGCTAAGTACAGTCTTGGGA |
| | GTCTCGATACTGAATCTTGGACAAAAGAAATACAC |
| | CAAGACAACATACTGGTGGGATGGGCTCCAATCAT |
| | CCGACGATTTTGCTCTCATAGTGAATGCACCAAAC |
| | CATGAAGGAATACAAGCAGGAGTGGACAGATTCTA |
| | CAGGACCTGCAAATTAGTGGGAATCAACATGAGCA |
| | AAAAGAAGTCCTATATAAATAAGACAGGGACATTT |
| | GAATTCACAAGCTTTTTTTATCGCTATGGATTTGT |
| | GGCTAATTTTAGCATGGAGCTACCCAGCTTTGGAG |
| | TGTCTGGAGTAAATGAATCAGCTGACATGAGTATT |
| | GGAGTAACAGTGATAAAGAACAACATGATAAACAA |
| | TGACCTTGGACCTGCAACGGCTCAGATGGCTCTTC |
| | (SEQ ID NO: 53) |
| FluB gBlock | gaaatTAATACGACTCACTATAgggCAAGCAAAAC |
| | AAAAAGACTAAAGGCCCAAATACCTTGTCCTGATC |
| | TGTTTAGTATACCATTAGAAAGATATAATGAAGAA |
| | ACAAGGGCAAAATTGAAGAAGCTAAAACCATTCTT |
| | CAATGAAGAAGGAACTGCATCTTTGTCACCTGGGA |
| | TGATGATGGGAATGTTTAATATGCTATCTACCGTG |
| | TTGGGAGTAGCTGCACTAGGTATCAAGAACATTGG |
| | AAACAAAGAATACCTATGGGATGGACTGCAATCTT |
| | CTGATGATTTTGCTCTATTTGTTAATGCAAAGGAT |
| | GAAGAAACATGTATGGAAGGAATAAACGACTTTTA |
| | CCGAACATGTAAATTATTGGGAATAAACATGAGCA |
| | AAAAGAAAAGTTACTGTAATGAGACTGGAATGTTT |
| | GAATTTACAAGCATGTTCTACAGAGATGGATTTGT |
| | ATCTAATTTTGCAATGGAACTCCCTTCGTTTGGGG |
| | TTGCTGGAGTAAATGAATCAGCAGATATGGCAATA |
| | (SEQ ID NO: 54) |
| HMPV gBlock | gaaatTAATACGACTCACTATAgggGAGAAGACCA |
| | AGGGTGGTATTGTCAGAATGCAGGGTCAACTGTTT |
| | ACTACCCAAATGAGAAAGACTGTGAAACAAGAGGA |
| | GACCATGTCTTTTGCGACACAGCAGCAGGAATTAA |
| | TGTTGCTGAGCAATCAAAGGAGTGCAACATCAACA |
| | TATCCACTACAAATTACCCATGCAAAGTCAGCACA |
| | GGAAGACATCCTATCA (SEQ ID NO: 55) |
| HRSV gBlock | gaaatTAATACGACTCACTATAgggGGGGCAAATA |
| | TGGAAACATACGTGAACAAACTTCACGAAGGCTCC |
| | ACATACACAGCTGCTGTTCAATACAATGTCCTAGA |
| | AAAAGACGATGATCCTGCATCACTTACAATATGGG |
| | TGCCCATGTTCCAATCATCCATGCCAGCAGATTTA |
| | CTTATAAAAGAACTAGCTAATGTCAACATACTAGT |
| | GAAACAAATATCCACA (SEQ ID NO: 56) |
| RNaseP gBlock | gaaatTAATACGACTCACTATAgggTCCTTGCAGG |
| | TGGCTGCCAATACCTCCACCGTGGAGCTTGTTGAT |
| | GAGCTGGAGCCAGAGACCGACACACGGGAGCCACT |
| | GACTCGGATCCGCAACAACTCAGCCATCCACATCC |
| | GAGTCTTCAGGGTCACACCCAAGTAATTGAAAAGA |
| | CACTCCTCCACTTATCCCCTCCGTGATATGGCTCT |
| | TCGCATGCTGAGTACTGGACCTCGGACCAGAGCCA |
| | TGTAAGAAAAGGCCTGTTCCCTGGAAGCCCAAAGG |
| | ACTCTGCATTGAGGGTGGGGGTAATTGTCTCTTGG |
| | TGGCCCAGTTAGTGGGCCTTCCTGA |
| | (SEQ ID NO: 57) |

Bead Preparation and Coupling for bbLuc

### Version Zero (V0) HiBiT and LgBiT Beads

HiBiT peptides were ordered from Promega as HaloTag protein conjugates (Promega #N3010), or custom-ordered through GenScript Inc. as a peptide with a leading glycine-serine linker (GSSGGSSG-VSGWRLFKKIS (SEQ ID NO: 58)) with either an N-terminal azido-lysine modification (for SPAAC) or with an N-terminal maleimide modification (for maleimide-thiol reactions).

V0 HiBiT-beads were prepared by mixing 80 nM of Halo-Tag-HiBiT with biotinylated poly-U (with 7U) HaloLigand (comprising chloroalkane) and incubated. The biotin-polyU-HiBiT was added to Streptavadin coated beads (Dynabeads M-280) (FIG. 25).

Evaluation of V0 HiBiT beads: V0 HiBiT beads demonstrated luminescence activity in solution with V0 LgBiT-beads (FIG. 26A). However, when Cas13 and RNase A were each used to cleave the polyU-HiBiT, followed by magnetic separation (FIG. 26B), a large amount of uncleaved poly-HiBiT was observed for Cas13 as compared to RNaseA, indicating the possibility that 7U may be too small. Changing the HiBiT linker length alone was ineffective to increase Cas13 cleavage (FIG. 27).

V0 LgBiT beads were prepared by coupling LgBiT protein to magnetic nanoparticles using antibodies. Two types of antibody-based coupling were used: Dynabeads Protein G (FIG. 28A) and Dynabeads M-270 epoxy. The V0 LgBiT beads work in a one-bead assay in which HiBiT is in solution (FIG. 28B). A full assay using LgBiT-beads and HiBiT-beads yielded a signal-to-noise ratio of ˜1.5 (FIG. 29). Other factors tested included: Oligo lengths (LgBiT was coupled to 7U vs. 21U linkers); Wash Buffers (NP-40, glycerol, tween); Bead Surface Density of HiBiT or LgBiT); Concentration of beads in solution (FIG. 30). A maximal signal-to-noise ratio of about 50 was achieved.

### Version One (V1) HiBiT and LgBiT Beads

For bead coupling, M270 Dynabeads were removed from stock and washed using magnetic separation three times with 1 minute incubations in 1×BW with Tween (5 mM Tris-HCl (pH 7.5), 0.5 mM EDTA, 1 M NaCl, 0.0125% Tween-20). After washing, beads were resuspended in twice the volume of 2× Wash Buffer with Tween (10 mM Tris-HCl (pH 7.5), 1 mM EDTA, 2 M NaCl, 0.025% Tween-20). HiBiT peptides were ordered from Promega as HaloTag protein conjugates (Promega #N3010), or custom-ordered through GenScript Inc. as a peptide with a leading glycine-serine linker (GSSGGSSG-VSGWRLFKKIS (SEQ ID NO: 58)) with either an N-terminal azido-lysine modification (for SPAAC) or with an N-terminal maleimide modification (for maleimide-thiol reactions). 0178 V1 HiBiT peptides were prepared by attaching biotinylated RNA linkers (Table 1) to the HiBiT peptides using either SPAAC (e.g., azide-alkyne for copper free SPAAC) or maleimide-thiol coupling. SPAAC coupling was done at room temperature overnight with a 3:1 ratio of azide-conjugated peptide to DBCO-conjugated oligo; maleimide-thiol coupling was done using a 20:1 ratio of maleimide-conjugated peptide to thiolated oligo left at room temperature for two hours, with an additional overnight incubation at 4 C. Cas13 cleavage of the biotinylated linker-HiBiT peptide was determined to be successful (FIG. 31). V1 LgBiT-beads were prepared by combining HaloTag-LgBiT protein (Promega, #CS1967B01) and HaloLigand-Biotin (Promega #G8281) in 1×PBS at a 1:1.5 stoichiometric ratio (with HaloTag-LgBiT protein concentration of 80 nM unless otherwise noted). Following a 30-minute incubation period on a rotation and another round of bead washing as above, LgBiT-beads were resuspended in Tween buffer (1000 uL PBS/0.05% Tween 20/0.1% BSA) and HiBiT-beads were resuspended in 1×TEL buffer (0.0125% Tween: 5 mM Tris-HCl (pH 7.5), 0.5 mM EDTA, 10 mM NaCl, 0.0125% Tween-20), followed by storage at 4 C.

Fluorescence and Luciferase-Based Cas13 Assays for bbLuc Development

Fluorescence-based reactions were conducted as previously described in (Myhrvold et al. 2018; Barnes et al. 2020; Welch et al. 2022; Arizti-Sanz et al. 2020; Ackerman et al. 2020; Arizti-Sanz et al. 2022) as a point of comparison for luminescent-based reactions and used a polyU FAM reporter. RPA primers and crRNa were designed and selected as described previously ((Myhrvold et al. 2018; Barnes et al. 2020; Welch et al. 2022; Arizti-Sanz et al. 2020; Ackerman et al. 2020; Arizti-Sanz et al. 2022)). (FIGS. 32, 33). 0180 For amplification-free assays, Leptotrichia wadei Cas13a (LwaCas13a) protein was first resuspended to 465.7 nM in 1×SB (50 mM Tris-HCl pH 7.5, 600 mM NaCl, 2 mM DTT, 5% glycerol). A master mix was created with the following reagents: 1×CB buffer (40 mM Tris-HCl (pH 7.5), 1 mM DTT), 22.5 nM crRNA, 2U/uL RNase Inhibitor Murine (NEB #M0314), 46.6 nM LwaCas13a (GenScript), and 40 mM of polyU FAM or 20 ug/uL LgBiT and 20 ug/uL HiBiT beads. In luciferase reactions, beads were washed in 1×TEL buffer using a process similar to that outlined above (1×TEL buffer: 5 mM Tris-HCl (pH 7.5), 0.5 mM EDTA, 10 mM NaCl, 0.0125% Tween) before being added to the final mix. While the final reaction concentrations of bead bound LgBiT and HiBiT were 80 nM and 300 nM, respectively, these concentrations were varied in experiments as described above to identify these optimal conditions. Fluorescent or luminescent kinetics were measured at 37C on a Biotek Cytation 5 plate reader using a 384-well Low Flange White Flat Bottom Polystyrene NBS Microplate (3574) or Corning® 384 well microplate (3821), respectively.

For amplification reactions (SHINE), the reaction was done as previously shown in (Myhrvold et al. 2018; Barnes et al. 2020; Welch et al. 2022; Arizti-Sanz et al. 2020; Ackerman et al. 2020; Arizti-Sanz et al. 2022). LwaCas13a protein was first resuspended to 2250 nM in 1×SB (50 mM Tris-HCl pH 7.5, 600 mM NaCl, 2 mM DTT, 5% glycerol). The master mix was created in 1×SHINE buffer (20 mM HEPES pH 8.0, 60 mM KCl, 5% PEG-8000) included 45 nM LwaCas13a protein, 1U/uL RNase Inhibitor Murine (NEB #M0314), 2 mM of each rNTP (NEB #N0450), 1 U/uL NextGen T7 RNA polymerase, 2U/uL Invitrogen SuperScript IV (SSIV) reverse transcriptase (Thermo Fisher Scientific #18090010), 0.1 U/uL RNase H (NEB #M0297S), 14 nM magnesium acetate (Millipore Sigma #63052), 140 nM RPA primers, 22.5 nM crRNA, and for fluorescence SHINE, 40 nM polyU FAM reporter. The reaction was created in reaction units of 107.5 uL, with one RPA pellet (TwistDx #TABASO3KIT) per reaction unit. 0182 In the case of luciferase reactions, beads were washed as above in 1×TEL buffer then resuspended in 5 mM HEPES buffer, pH 8.0 with 80 nM furimazine. To reduce variability caused by reaction viscosity, the reaction master mix was assembled as above and aliquoted to a final reaction tube prior to addition of 20 ug/uL LgBiT and HiBiT beads to a final reaction mixture of 19 uL. Finally, 1 uL target or sample was added to the reaction before measurement for three hours at 37 C in a Biotek Cytation 5.

Iterative optimization of the reaction was done via modification of reagent and bead concentration as described in each experiment. Optimal conditions that produced the lowest limit of detection were incorporated into the final protocol as described. In each optimization experiment, the reaction component that was changed is outlined in the results or figures above. The following conditions remained constant across experiments: 45 nM LwaCas13a protein resuspended in 1×SB (such that resuspended protein is at 2.26 uM), 1 U/uL murine RNase inhibitor, and 2 mM of each rNTP.

For all fluorescent data shown (including curves), fluorescence values were normalized across condition by dividing timepoint data by the mean NTC signal at the first collected timepoint. For luminescence data for amplification-free conditions (including curves) and timepoint curves for SHINE, luminescence values were normalized across condition by dividing timepoint data by the mean NTC signal at the first collected timepoint. For luminesce SHINE data, the larger kinetic complexity precluded the use of a single timepoint to determine a positive/negative call. As such, calls were shown as luminescence ratios, an overall measure of signal across the timepoint curve was determined. This was done by first aligning experimental and NTC condition slopes (as computed between the timepoint nearest to 12 minutes and its subsequent timepoint) by dividing experimental condition by the NTC slope, and next by finding the ratio of the sum of intensities across the experimental and NTC conditions. Patient samples were determined positive with a signal threshold>1.35.

Bead Preparation and Coupling for bbCARMEN

Streptavidin-coated polystyrene beads (Spherotech, no. SVP-200-4) were washed and stored in a binding and washing buffer (2×BW Buffer: 10 mM Tris-HCL pH 7.5, 1 mM EDTA, and 2 M NaCl). To prepare beads for BSA coating, 1 mL of beads was washed with 1 mL of 1×BW Buffer three times before being resuspended in 2 mL of 2× buffer and 2 mL BSA (4 mg/mL) (NEB #B9000). Beads were BSA blocked for 3 hours on a rotating stand at room temperature before washing with 1×BW Buffer twice and resuspended in twice the original volume of 2×BW Buffer. BSA blocked beads were stored at 4C until use at a 2.5 ug/uL bead concentration. 0186 crRNA and dye coupling were split into two separate steps. First, 32 nM of desired crRNA was mixed with BSA blocked beads in a 1:1 ratio and incubated at room temperature for 15 minutes. After the coupling incubation, crRNA beads were washed with 1×BW Buffer once before resuspending in the original volume of beads with 2×BW Buffer. An equal volume of pre-mixed color-coding dyes (see “color code construction and validation” methods for ratios) were added to the corresponding crRNA bead and incubated at room temperature for 15 minutes. Color-coded crRNA beads were washed six times with IX BW buffer and then resuspended in IX TEL buffer (5 mM Tris-HCl pH 7.5, 0.5 mM EDTA, 10 mM NaCl) to have a final bead concentration of 25 ug/uL. crRNA beads were stored at 4C until use and washed twice prior to pooling for each experiment. Equal volumes of beads were pooled together the day of an experiment and incubated in a bbCARMEN Wash Buffer (Cas13 detection master mix without Cas13, 10× Cleavage Buffer, and viral target) for 60 minutes and washed twice with 1×BW Buffer. All washes were accomplished by spinning at 15000 rcf on a tabletop centrifuge and discarding the supernatant. Non-BSA blocked beads required spin times of 3.5 minutes while BSA blocked beads required 1.5 minutes.

Flow Cell Design and Fabrication for bbCARMEN

Flow cell dimensions were designed in AutoCAD (AutoDesk) and optimized by empirical testing to increase sample size and loading speed. In order to be compatible with existing imaging instruments, the size of a standard microscope slide (25×75 mm) was selected. The optimal lane geometry was achieved by maximizing the number of droplets captured in a single lane image field of view. To allow for easy loading, eight 10.5×5.8 mm lanes were spaced out on the 75 mm long flow cell with inlet spacing of 9 mm for compatibility with 8-channel multichannel pipettes. Standard size flow cells contain two rows of eight for 16 samples per device. Increasing flow cell dimensions to 50×75 mm enabled 32 lane imaging per device.

All flow cells were fabricated with acrylic, a single layer of double-sided clear film tape, and hydrophobic treated glass slides. In brief, 12 inch×12 inch cast acrylic sheets (V4 inch or ⅛ inch, clear) were purchased from Amazon (Small Parts, no. B004N1JLI4) and were cut using an Epilog Fusion M2 laser cutter (60 W), producing an acrylic cover with inlets and outlets. Sheets of clear film tape were cut on the laser cutter to provide the geometry of the lanes. Untreated glass slides were treated with Aqualpel from Amazon (Aquapel, no. 2PACK_A) to create a hydrophobic surface. For assembly of the both the 16 and 32 lane flow cells, the clear tape was first adhered to the Aquapel treated glass slide and then to the acrylic cover. Flow cells were stored in plastic bags at room temperature until use.

Single-Step Amplification for bbCARMEN

All targets for RVP2.0 were amplified using the QIAGEN OneStep RT-PCR Mix. A total reaction volume of 50 μl was used with some modifications to the manufacturer's recommended reagent volumes, specifically a 1.25× final concentration of OneStep RT-PCR buffer, 2× more QIAGEN enzyme mix and 20% RNA input. For optimal amplification, final viral primer concentrations varied, with SARS-CoV2, HCoV_NL63, HCoV_OC43, HPIV3, and HMPV primer concentrations at 300 nM, HCoV_HKU1 and HRSV at 600 nM, FluA and FluB at 480 nM, and RNase P at 100 nM. The following thermal cycling conditions were used: (1) reverse transcription at 50° C. for 30 min; (2) initial PCR activation at 95° C. for 15 min; and (3) 40 cycles at 94° C. for 30 s, 56° C. for 30 s and 72° C. for 30 s.

Cas13 Detection in bbCARMEN

Detection assays were performed with 45 nM purified LwaCas13a, 0.5 ug/uL of pooled crRNA beads, 500 nM quenched fluorescent RNA reporter, 1 μl murine 40,000 units/mL RNase inhibitor (New England Biolabs) in nuclease assay buffer (40 mM Tris-HCl, 60 mM NaCl, pH 7.3) with 1 mM ATP, 1 mM GTP, 1 mM UTP, 1 mM CTP and 0.6 μl T7 polymerase mix (Lucigen).

Emulsification, Loading, and Imaging in bbCARMEN Flow Cells.

For emulsification, detection samples (10 uL) were mixed with 2% 008-fluorosurfactant (RAN Biotechnologies) in fluorous oil (3M 7500, 35 μl) in a 96 well plate. Plates were sealed and physically shaken vertically for up to 30 seconds and then spun down for 15 seconds.

For loading, 30 μL of excess oil was removed from each emulsion before 9 μL of droplets were loaded into a 16 or 32 lane bbCARMEN flow cell. The background negative control was computed by analyzing fluorescence signal from droplets containing a scrambled crRNA sequence attached to a color-coded bead. Flow cells were sealed with a PCR film to prevent evaporation of samples.

All bbCARMEN flow cells were imaged on a Nikon TI2 microscope equipped with an automated stage (Ludl Electronics, Bio Precision 3 LM), LED light source (Lumencor, Sola), and camera (Hamamatsu, Orca Flash4.0, C11440, sCMOS) using a 2× objective (Nikon, MRD00025). The following filter cubes were used for imaging: Alexa Fluor 405: Semrock LED-DAPI-A-000; Alexa Fluor 555: Semrock SpGold-B; Alexa Fluor 594: Semrock 3FF03-575/25-25+FF01-615/24-25; and Alexa Fluor 647: Semrock LF635-B. During imaging, the microscope condenser was tilted back to reduce background fluorescence in the 488 chan-nel. Unless otherwise specified, all flow cells were imaged three times over the course of 60 minutes, with an incubation at 37C between T30 and T60.

Automated bbCARMEN Data Analysis

CellProfiler (Stirling et al. 2021) and a custom Jupyter notebook were used to automate image analysis for bbCARMEN (Lamprecht, Sabatini, and Carpenter 2007). First, beads were identified and measured in red, yellow, green, and blue channels using a CellProfiler pipeline. Briefly, images were illumination corrected by subtracting an approximation of image background from each channel. Then, bleedthrough between color channels was computationally compensated for by image subtraction. The corrected images were then masked to exclude the edges of the wells where droplets piled up. Beads were filtered by shape (solidity, eccentricity) to exclude debris and by number of neighbors to exclude beads that were very close to other beads. Beads were also associated with droplets and excluded if a droplet contained multiple beads. The object mask for each accepted bead was expanded 5 pixels and the original bead area subtracted from this to form a ‘donut’ shape in which intensity in the droplet blue channel was measured. For each bead, Applicant calculated normalized intensity measurements for each (red, yellow, green) channel by dividing the mean intensity measurement for each channel by the sum of mean intensities across all 3 channels. Finally, beads were also tracked across images taken at different timepoints using linear assignment problem (LAP) framework (Jaqaman et al. 2008).

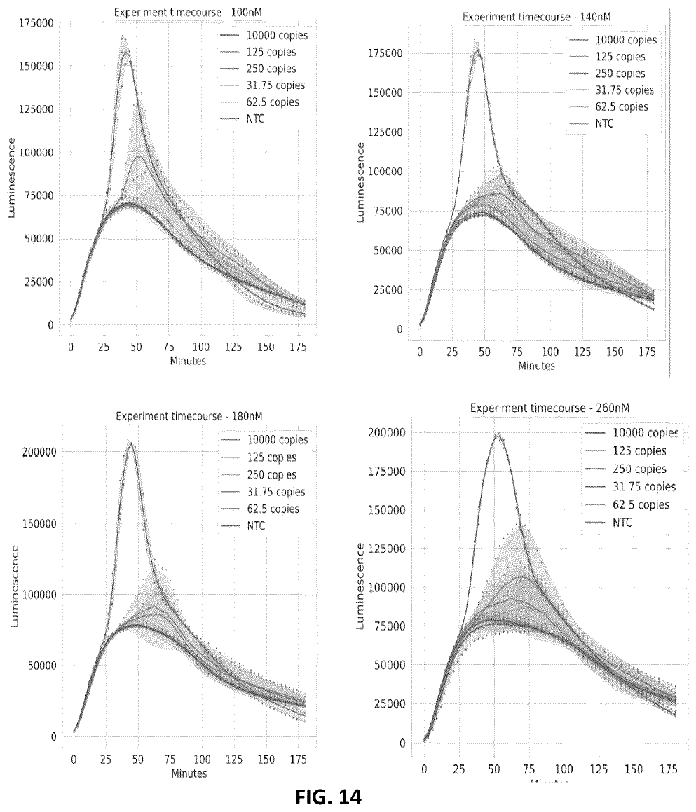

CellProfiler measurements were used for bead classification and FAM fluorescence measurement in a separate downstream analysis jupyter notebook. Beads were clustered using normalized red, green, and yellow intensity measurements for each bead using k-means clustering. Results are displayed in a ternary plot showing each bead's intensity in green, yellow, and red channels and beads are colored by cluster (Supplemental FIG. 14). Each cluster was then associated with a virus from the panel by measuring the distance from its measured centroid to the default centroid locations (provided by an external file) and selecting the label of the closest default cluster centroid. In this process, additional QC plots and metrics are also generated and used to assess data quality. Finally, bead donut median blue channel intensities were used to classify samples as positive or negative for each virus in the virus panel using a threshold based on either fold difference from negative control beads (in the same well) and/or exceeding a number of standard deviations above the median intensity of negative control beads. Tracked bead blue channel fluorescence was also plotted over time to observe kinetics of FAM fluorescence.

### SCoV2 RT-qPCR Protocol

To detect the presence of SARS-CoV-2 RNA, the extracted RNA samples underwent testing using the CDC's SARS-CoV-2 RT-qPCR assay (2019-nCoV CDC EUA kit, IDT) targeting the N1 and RP regions. The cycling conditions for the RT-qPCR were as follows: an initial hold at 25° C. for 2 min, followed by reverse transcription at 50° C. for 15 min, polymerase activation at 95° C. for 2 min, and 45 cycles of denaturation at 95° C. for 3 s, and annealing/elongation at 55° C. for 30 s. The RT-qPCR analysis was performed using a QuantStudio 6 instrument from Applied Biosystems, and the data were analyzed using the Standard Curve module of the Applied Biosystems analysis software.

### REFERENCES

Ackerman, Cheri M., Cameron Myhrvold, Sri Gowtham Thakku, Catherine A. Freije, Hayden C. Metsky, David K. Yang, Simon H. Ye, et al. 2020. “Massively Multiplexed Nucleic Acid Detection with Cas13.” Nature 582 (7811): 277-82.
Anderson, Steve, Phil Wakeley, Guy Wibberley, Kath Webster, and Jason Sawyer. 2011. “Development and Evaluation of a Luminex Multiplex Serology Assay to Detect Antibodies to Bovine Herpes Virus 1, Parainfluenza 3 Virus, Bovine Viral Diarrhoea Virus, and Bovine Respiratory Syncytial Virus, with Comparison to Existing ELISA Detection Methods.” Journal of Immunological Methods 366 (1-2): 79-88.
Arizti-Sanz, Jon, A'doriann Bradley, Yibin B. Zhang, Chloe K. Boehm, Catherine A. Freije, Michelle E. Grunberg, Tinna-Solveig F. Kosoko-Thoroddsen, et al. 2022. “Simplified Cas13-Based Assays for the Fast Identification of SARS-CoV-2 and Its Variants.” Nature Biomedical Engineering 6 (8): 932-43.
Arizti-Sanz, Jon, Catherine A. Freije, Alexandra C. Stanton, Brittany A. Petros, Chloe K. Boehm, Sameed Siddiqui, Bennett M. Shaw, et al. 2020. “Streamlined Inactivation, Amplification, and Cas13-Based Detection of SARS-CoV-2.” Nature Communications 11 (1): 5921.
Barnes, Kayla G., Anna E. Lachenauer, Adam Nitido, Sameed Siddiqui, Robin Gross, Brett Beitzel, Katherine J. Siddle, et al. 2020. “Deployable CRISPR-Cas13a Diagnostic Tools to Detect and Report Ebola and Lassa Virus Cases in Real-Time.” Nature Communications 11 (1): 4131.
Bielefeld-Sevigny, Martina. 2009. “AlphaLISA Immunoassay Platform—the ‘No-Wash’ High-Throughput Alternative to ELISA.” Assay and Drug Development Technologies 7 (1): 90-92.
Center for Devices, and Radiological Health. n.d. “Policy for Coronavirus Disease-2019 Tests (revised).” U.S. Food and Drug Administration. FDA. Accessed Jun. 13, 2023. https://www.fda.gov/regulatory-information/search-fda-guidance-documents/policy-coronavirus-disease-2019-tests-revised?_cldee=Y21vbmtzQGFtY3Aub3Jn&recipientid=contact-06c36f12c97dea11 a2de000c2959e3d7-bcf37b12509546859fa8d3d78dbc227f.
Chen, Janice S., Enbo Ma, Lucas B. Harrington, Maria Da Costa, Xinran Tian, Joel M. Palefsky, and Jennifer A. Doudna. 2018. “CRISPR-Cas12a Target Binding Unleashes Indiscriminate Single-Stranded DNase Activity.” Science 360 (6387): 436-39.
Chu, Victoria T., Noah G. Schwartz, Marisa A. P. Donnelly, Meagan R. Chuey, Raymond Soto, Anna R. Yousaf, Emily N. Schmitt-Matzen, et al. 2022. “Comparison of Home Antigen Testing With RT-PCR and Viral Culture During the Course of SARS-CoV-2 Infection.” JAMA Internal Medicine 182 (7): 701-9.
Clark, lain C., Kristina M. Fontanez, Robert H. Meltzer, Yi Xue, Corey Hayford, Aaron May-Zhang, Chris D'Amato, et al. 2023. “Microfluidics-Free Single-Cell Genomics with Templated Emulsification.” Nature Biotechnology, March. https://doi.org/10.1038/s41587-023-01685-z.
Dixon, Andrew S., Marie K. Schwinn, Mary P. Hall, Kris Zimmerman, Paul Otto, Thomas H. Lubben, Braeden L. Butler, et al. 2016. “NanoLuc Complementation Reporter Optimized for Accurate Measurement of Protein Interactions in Cells.” ACS Chemical Biology 11 (2): 400-408.
East-Seletsky, Alexandra, Mitchell R. O'Connell, Spencer C. Knight, David Burstein, Jamie H. D. Cate, Robert Tjian, and Jennifer A. Doudna. 2016. “Two Distinct RNase Activities of CRISPR-C2c2 Enable Guide-RNA Processing and RNA Detection.” Nature 538 (7624): 270-73.
Eckbo, Eric J., Kerstin Locher, Melissa Caza, Lisa Li, Valery Lavergne, and Marthe Charles. 2021. “Evaluation of the BioFire® COVID-19 Test and Respiratory Panel 2.1 for Rapid Identification of SARS-CoV-2 in Nasopharyngeal Swab Samples.” Diagnostic Microbiology and Infectious Disease 99 (3): 115260.
Fan, Frank, and Keith V. Wood. 2007. “Bioluminescent Assays for High-Throughput Screening.” Assay and Drug Development Technologies 5 (1): 127-36.
Fozouni, Parinaz, Sungmin Son, Maria Diaz de Le6n Derby, Gavin J. Knott, Carley N. Gray, Michael V. D'Ambrosio, Chunyu Zhao, et al. 2021. “Amplification-Free Detection of SARS-CoV-2 with CRISPR-Cas13a and Mobile Phone Microscopy.” Cell 184 (2): 323-33.e9.
Fulton, R. Jerrold, Ralph L. McDade, Perry L. Smith, Laura J. Kienker, and John R. Kettman. 1997. “Advanced Multiplexed Analysis with the FlowMetrix™ System.” Clinical Chemistry 43 (9): 1749-56.
Goodwin, Sara, John D. McPherson, and W. Richard McCombie. 2016. “Coming of Age: Ten Years of next-Generation Sequencing Technologies.” Nature Reviews. Genetics 17 (6): 333-51.
Gootenberg, Jonathan S., Omar O. Abudayyeh, Jeong Wook Lee, Patrick Essletzbichler, Aaron J. Dy, Julia Joung, Vanessa Verdine, et al. 2017. “Nucleic Acid Detection with CRISPR-Cas13a/C2c2.” Science 356 (6336): 438-42.
Jaqaman, Khuloud, Dinah Loerke, Marcel Mettlen, Hirotaka Kuwata, Sergio Grinstein, Sandra L. Schmid, and Gaudenz Danuser. 2008. “Robust Single-Particle Tracking in Live-Cell Time-Lapse Sequences.” Nature Methods 5 (8): 695-702.
Knox, Alexandra, and Travis Beddoe. 2021. “Isothermal Nucleic Acid Amplification Technologies for the Detection of Equine Viral Pathogens.” Animals: An Open Access Journal from MDPI 11 (7). https://doi.org/10.3390/anil1072150.
Lamprecht, Michael R., David M. Sabatini, and Anne E. Carpenter. 2007. “CellProfiler: Free, Versatile Software for Automated Biological Image Analysis.” BioTechniques 42 (1): 71-75.
Liu, Tina Y., Gavin J. Knott, Dylan C. J. Smock, John J. Desmarais, Sungmin Son, Abdul Bhuiya, Shrutee Jakhanwal, et al. 2021. “Accelerated RNA Detection Using Tandem CRISPR Nucleases.” Nature Chemical Biology 17 (9): 982-88.
Los, Georgyi V., Lance P. Encell, Mark G. McDougall, Danette D. Hartzell, Natasha Karassina, Chad Zimprich, Monika G. Wood, et al. 2008. “HaloTag: A Novel Protein Labeling Technology for Cell Imaging and Protein Analysis.” ACS Chemical Biology 3 (6): 373-82.
Myhrvold, Cameron, Catherine A. Freije, Jonathan S. Gootenberg, Omar O. Abudayyeh, Hayden C. Metsky, Ann F. Durbin, Max J. Kellner, et al. 2018. “Field-Deployable Viral Diagnostics Using CRISPR-Cas13.” Science 360 (6387): 444-48.
Pardee, Keith, Alexander A. Green, Melissa K. Takahashi, Dana Braff, Guillaume Lambert, Jeong Wook Lee, Tom Ferrante, et al. 2016. “Rapid, Low-Cost Detection of Zika Virus Using Programmable Biomolecular Components.” Cell 165 (5): 1255-66.
Park, Bum Ju, Man Seong Park, Jae Myun Lee, and Yoon Jae Song. 2021. “Specific Detection of Influenza A and B Viruses by CRISPR-Cas12a-Based Assay.” Biosensors 11 (3): 88.
Schwinn, Marie K., Thomas Machleidt, Kris Zimmerman, Christopher T. Eggers, Andrew S. Dixon, Robin Hurst, Mary P. Hall, Lance P. Encell, Brock F. Binkowski, and Keith V. Wood. 2018. “CRISPR-Mediated Tagging of Endogenous Proteins with a Luminescent Peptide.” ACS Chemical Biology 13 (2): 467-74.
Stirling, David R., Madison J. Swain-Bowden, Alice M. Lucas, Anne E. Carpenter, Beth A. Cimini, and Allen Goodman. 2021. “CellProfiler 4: Improvements in Speed, Utility and Usability.” BMC Bioinformatics 22 (1): 433.
Tung, Jack K., Ken Berglund, Claire-Anne Gutekunst, Ute Hochgeschwender, and Robert E. Gross. 2016. “Bioluminescence Imaging in Live Cells and Animals.” Neurophotonics 3 (2): 025001.
   Ullman, E. F., H. Kirakossian, S. Singh, Z. P. Wu, B. R. Irvin, J. S. Pease, A. C. Switchenko, J. D. Irvine, A. Dafforn, and C. N. Skold. 1994. “Luminescent Oxygen Channeling Immunoassay: Measurement of Particle Binding Kinetics by Chemiluminescence.” Proceedings of the National Academy of Sciences of the United States of America 91 (12): 5426-30.
Welch, Nicole L., Meilin Zhu, Catherine Hua, Juliane Weller, Marzieh Ezzaty Mirhashemi, Tien G. Nguyen, Sreekar Mantena, et al. 2022. “Multiplexed CRISPR-Based Microfluidic Platform for Clinical Testing of Respiratory Viruses and Identification of SARS-CoV-2 Variants.” Nature Medicine 28 (5): 1083-94.

### Example 2—Molecular Beacon Split-Luciferase Reporter System

### Version Zero (V0)

Luminescent molecular beacon comprises a Cas13-cleavable RNA linker connecting a LgBiT protein and a DrkBiT protein. The DrkBiT protein and the LgBiT protein are designed to form a complex which lacks luciferase activity. In the presence of HBiT, luciferase substrate, an activated Cas13, and optionally additional free DrkBiT, Cas13 cleavage of the RNA linker allow formation of a luminescent complex between the LgBiT protein and the HgBiT protein. (FIG. 34). The addition of free DrkBiT to luminescent molecular beacon [LgBiT coupled to DrkBiT] reduces the luminescent signal (FIGS. 35A-35B, 36).

A V0 molecular beacon as prepared by combining a Cas13-cleavable RNA linker comprising a protected thiol end group and an amine end group (Thiol-RNA-Amine) with a HaloLigand comprising a N-hydroxysuccinamide (NHS) ester end group to form a Cas13-cleavable RNA linker with a protected thiol end group and a Halo-Ligand end group (Thiol-RNA-HaloLigand). The filter cutoff of the Thiol-RNA-HaloLigand was 3 kD (with the Cas13-Cleavable RNA linker from 7 kD to 20 kD and the HaloLigand NHS ester at 500 D). After deprotection of the thiol group, a DrkBiT connected by a glycine group to a maleimide group (DrkBiT-glycine-Maleimide) was reacted to the Thiol-RNA-HaloLigand. The size-select spin kit had a 30-50 kD cutoff (DrkBiT-glycine is ˜2 kD). Thiol-maleimide conjugation generated the V0 Molecular Beacon (DrkBiT-glycine-Maleimide-Thiol-RNA-HaloLigand). Reaction of the conjugate with HaloTag labeled LgBiT (HaloTag/LgBiT) formed the V0 molecular beacon (DrkBiT-glycine-Maleimide-Thiol-RNA-HaloLigand-HaloTag/LgBiT).

An V0 molecular beacon assay solution was prepared comprising: (a) molecular beacon (5 nM) [LgBiT coupled to DrkBiT] and (b) HiBiT (25 nM) with DrkBiT in solution (depending on filtering efficacy, should be 0 nM, perhaps up to −30 nM). A SHERLOCK master mix is prepared as normal, replacing Poly-U FAM with luminescent molecular beacon. Variations may include: Different linker lengths: 25U, 35U, 45U, 60U, 35T, 60T; or Rnase/Dnase condition, 1e9, 1e8, 1e7, NTC.

### Version One (V1)

Instead of having one contiguous, covalently-linked molecule which includes DrkBiT, cleavable RNA, and LgBiT, these three species can be connected together using DNA hybridization; i.e., DrkBiT attached to the “left primer”, the LgBiT attached via HaloLigand to the “right primer”, and the cleavable RNA linker (“backbone”) which connects the species via DNA hybridization. DrkBiT attachment to “left primer” may be via SPAAC or maleimide-thiol. An example splint design may comprise the sequences shown in Table 2.

TABLE 2

| Species | Sequence |
|---|---|
| Cleavable | 5′CACGAAGTCAACTCTACAGGUUUUUU |
| RNA | UUUUUUUUUUUUUUUUUUUUUUUUUUUU |
| backbone | UUUUCACCTAGCGTAATAGTGAAGAG |
| | (SEQ ID NO: 59) |
| Left primer | 3′(DBCO or Thiol)-GTGCTTCAGTTG |
| | AGATGTCC′ (SEQ ID NO: 60) |
| Right primer | 3′-GTGGATCGCATTATCACTTCTC-5′- |
| | (HaloLigand) (SEQ ID NO: 61) |
| Alternate | 5′ adapter--38U--3′ adapter |
| backbones | 5′ adapter--60U--3′ adapter |
| | 5′ adapter--12TTA 14U 12TTA--3′ |
| | adapter |
| | 5′ adapter--15TTA 8U 15TTA--3′ |
| | adapter |
| | 5′ adapter--8U 30TTA--3′ adapter |
| | 5′ adapter--30TTA 8U--3′ adapter |
| | 5′ adapter--78U--3′ adapter |
| | 5′ adapter--15TTA 38U15TTA--3′ |
| | adapter |

Luminescent molecular beacons cleaved generate higher luminescence that the background NTC luminescence when cleaved by RNase A (FIGS. 36, 37A-37B) or Cas13 (FIGS. 38A-38B).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A split luciferase reporter system, comprising:
a first reporter protein subunit coupled to first bead or a masking agent via a first oligonucleotide linker capable of being cleaved by Cas nuclease activity, optionally, Cas collateral nuclease activity;
a second reporter protein subunit optionally coupled to a second bead via a second linker, optionally, a second oligonucleotide linker,
wherein the first and second reporter protein subunits can form a complex to generate a detectable luminescent signal, and
wherein the masking agent or the bead prevents generation of the detectable luminescent signal.

2. The split luciferase reporter system of claim 1, wherein the first oligonucleotide linker and/or the second linker is selected from the group consisting of a DNA sequence, an RNA sequence, or any combination thereof,
wherein the first oligonucleotide linker and/or the second linker comprises at least 5, at least 6, at least 7, at least 8, at least 9, or at least 10 nucleotides, and is selected from an oligonucleotide of Table 1 or a derivative thereof.

3. (canceled)

4. (canceled)

5. The split luciferase reporter system of claim 1, further comprising a reacting reagent, wherein the reacting reagent is furimazine, an analogue thereof, or any combination thereof.

6. (canceled)

7. The split luciferase reporter system of claim 1, wherein the first protein subunit is HiBiT and the second protein subunit is LgBiT.

8. The split luciferase reporter system of claim 1, wherein the first bead and/or the second bead are selected from the group consisting of bovine serum albumin (BSA) coated beads, streptavidin coated beads, amine-functionalized beads, thiol-functionalized beads, agarose beads, polystyrene beads, magnetic beads, hydrophilic beads, and any combination thereof, and wherein, optionally, the beads are nanobeads or microbeads.

9. The split luciferase reporter system of claim 1, wherein the first oligonucleotide linker and/or the second linker connects to a corresponding protein subunit, a corresponding bead, and/or the masking construct, using:
a click chemistry connection, an amine coupling connection, a thiol coupling connection, or any combination thereof;
a Strain-Promoted Azide-Alkyne Click Chemistry (SPAAC) reaction connection, a maleimide-thiol reaction connection, an amine-N-hydroxysuccinamide reaction connection, or any combination thereof;
a biotin-streptavidin connection, or
an antibody-coupling connection; and/or a HaloTag-HaloLigand connection.

10. The split luciferase reporter system of claim 7, wherein the masking agent is a luminescence inactivator,
wherein the first protein subunit and the luminescence inactivator are in a catalytically inactive complex which does not generate a luminescent signal upon exposure to a reacting agent, and
wherein the catalytically inactive complex is disrupted upon Cas-cleavage of oligonucleotide linker, and
wherein the luminescence inactivator is a catalytically inactive third protein or peptide subunit, optionally DrkBiT.

11. (canceled)

12. (canceled)

13. The split luciferase reporter system of claim 10, wherein:
the first oligonucleotide linker covalently connects the first protein subunit and the luminescence inactivator; or
wherein the luminescence inactivator is connected to a 5′ oligonucleotide and the first protein subunit is connected to a 3′ oligonucleotide or vice versa,
wherein the 5′ and the 3′ oligonucleotides are capable of hybridizing with the Cas-cleavable oligonucleotide linker at positions 5′ and 3′, respectively, of the Cas-cleavable oligonucleotide sequence, such that the first protein or peptide subunit and the luminescence inactivator form a catalytically inactive bound complex upon hybridization to the Cas-cleavable oligonucleotide linker and
wherein the luminescence inactivator is connected to the 5′ or the 3′ oligonucleotide using a click chemistry connection; and/or
wherein the first protein subunit is connected to the 5′ or the 3′ oligonucleotide using a HaloLigand connection.

14. (canceled)

15. The split luciferase reporter system of claim 10, further comprising a luminescence inactivator that is not connected to the first protein subunit.

16. A detection system for detecting one or more target oligonucleotides in a sample, comprising:
a split luciferase reporter system of claim 1; and
a CRISPR-Cas system comprising
a Cas protein, optionally, selected from Type V Cas, Type VI Cas, or any combination thereof;
wherein the Cas protein, optionally, exhibits collateral DNA cleavage activity, collateral RNA cleavage activity, or any combination thereof, and
one or more guide molecules capable of forming a complex with the Cas protein and directing sequence-specific hybridization to one or more corresponding target oligonucleotide sequences,
wherein formation of the complex activates, the Cas protein's collateral RNA cleavage activity which results in the cleavage of cleaves the first and, optionally, the second oligonucleotide linker of the split luciferase reporter system, thereby generating a detectable luminescent signal indicating a presence of one or more of the target oligonucleotide sequences in the sample,
wherein the detection system, optionally, further comprises nucleic acid amplification reagents chosen from nucleic acid sequence-based amplification (NASBA) reagents, recombinase polymerase amplification (RPA) reagents, loop-mediated isothermal amplification (LAMP) reagents, strand displacement amplification (SDA) reagents, helicase-dependent amplification (HDA) reagents, nicking enzyme amplification reaction (NEAR) reagents, polymerase chain reaction (PCR) reagents, multiple displacement amplification (MDA) reagents, rolling circle amplification (RCA) reagents, ligase chain reaction (LCR) reagents, or ramification amplification method (RAM) reagents.

17. (canceled)

18. (canceled)

19. (canceled)

20. (canceled)

21. A method for detecting one or more target oligonucleotides in a sample, comprising:
contacting the sample with the detection system of claim 16 wherein the generation of the detectable luminescent signal indicates the presence of one or more of the target oligonucleotide sequences in the sample;
optionally, further comprising detecting the detectable luminescent signal,
wherein the detection occurs over a period of time from 25 minutes to 3 hours; and
optionally, applying the sample to a lateral flow immunochromatographic assay prior to or after the contacting step.

22. (canceled)

23. (canceled)

24. (canceled)

25. A color-coded bead multiplex CRISPR-Cas detection system for detecting one or more target oligonucleotides in a sample, comprising;
a) a Cas protein,
wherein the Cas protein exhibits collateral DNA cleavage activity, collateral RNA cleavage activity, or any combination thereof, and
wherein, optionally, the Cas protein is selected from Cas12, Cas13, or any combination thereof, and
b) a set of guide molecules capable of forming a complex with the Cas protein and directing sequence-specific hybridization to a set of target nucleotide sequences,
wherein each guide molecule is bound to a color-coded bead, that identifies the guide molecule and the corresponding target nucleotide sequence;
c) one or more detection constructs comprising a non-target sequence susceptible to the Cas protein's collateral cleavage activity,
wherein, the non-target sequence is selected from a DNA sequence, an RNA sequence, or any combination thereof; and
d) wherein complex formation activates the Cas protein's collateral activity which results in the cleavage of the non-target sequence, thereby generating a detectable signal indicating a presence of one or more of the target oligonucleotide sequences in the sample.

26. The color-coded bead multiplex CRISPR-Cas detection system of claim 25, wherein the color-coded beads are selected from the group consisting of bovine serum albumin (BSA) coated beads, streptavidin coated beads, agarose beads, polystyrene beads, magnetic beads, hydrophilic beads, or any combination thereof and
wherein the color-coded beads are optionally, nanobeads or microbeads;
wherein the guide molecules are, optionally, bound to the beads by a biotin-streptavidin connection; and
wherein the oligonucleotide-based detection constructs optionally comprise an oligonucleotide to which a detectable ligand and a masking component are attached,
wherein the detectable ligand is optionally a fluorophore and the masking component is a quencher molecule.

27. (canceled)

28. (canceled)

29. (canceled)

30. (canceled)

31. (canceled)

32. (canceled)

33. The color-coded bead multiplex CRISPR-Cas detection system of claim 25, wherein the detection CRISPR system further comprises nucleic acid amplification reagents optionally comprising nucleic acid sequence-based amplification (NASBA) reagents, recombinase polymerase amplification (RPA) reagents, loop-mediated isothermal amplification (LAMP) reagents, strand displacement amplification (SDA) reagents, helicase-dependent amplification (HDA) reagents, nicking enzyme amplification reaction (NEAR) reagents, PCR reagents, multiple displacement amplification (MDA) reagents, rolling circle amplification (RCA) reagents, ligase chain reaction (LCR) reagents, or ramification amplification method (RAM) reagents.

34. (canceled)

35. The color-coded bead multiplex CRISPR-Cas detection system of claim 25 further comprising a pool of droplets comprising a sample and the color-coded bead multiplex CRISPR-Cas detection system,
wherein each droplet comprises a single one of the color-coded beads, such that, detection of the detectable signal and identifying the color of the color-coded bead within one of the droplets indicates a presence in the sample of the target oligonucleotide sequence corresponding to the guide molecule identified by the color-coded bead in said droplet.

36. The color-coded bead multiplex CRISPR-Cas detection system of claim 35, wherein the pool of droplets is an oil-in-water emulsion,
wherein the oil is, optionally, a fluorous oil, and optionally the oil further comprises a surfactant that is a fluorosurfactant.

37. (canceled)

38. A color-coded bead multiplex CRISPR-Cas detection method for detecting a target sequence in a sample, comprising:
contacting a sample with a color-coded bead multiplex CRISPR-Cas detection system of claim 25, thereby forming a pre-mixture;
mixing the pre-mixture with a solvent immiscible with the pre-mixture, thereby forming a pool of droplets comprising the sample and the detection CRISPR system;
detecting each droplet generating a detectable signal and comprising a single one of the color-coded beads; and
identifying the color-code of the single bead within said droplet, thereby indicating a presence in the sample of the target sequence corresponding to the guide molecule identified by the single color-coded bead in said droplet.

39. The color-coded bead multiplex CRISPR-Cas detection method of claim 38, wherein the pool of droplets is an oil-in-water emulsion, wherein the oil is a fluorous oil, and optionally comprises a surfactant that is a fluorinated surfactant.

40. (canceled)

41. The color-coded bead multiplex CRISPR-Cas detection method of claim 38, wherein prior to the detecting steps, the method further comprises
loading the pool of droplets onto a flow cell, wherein the detecting steps are performed with a microscope; or
loading the pool of droplets onto a well plate, wherein the detecting steps are performed with a plate reader.


SEQUENCE LISTING (61 sequences)

SEQ ID NO: 1 - RNA, 14 nt, synthetic construct
  misc_feature 1..14: note = Synthetic
  misc_feature 1: note = 5' biotin tag
  misc_feature 14: note = HaloLigand modification
  source 1..14: mol_type = other RNA
tttttttttttttt

SEQ ID NO: 2 - RNA, 21 nt, synthetic construct
  misc_feature 1: note = 5' biotin tag
  misc_feature 21: note = HaloLigand modification
  source 1..21: mol_type = other RNA
ttttttttttttttttttttt

SEQ ID NO: 3 - RNA, 40 nt, synthetic construct
  misc_feature 1..40: note = Synthetic
  misc_feature 1: note = 5' biotin tag
  misc_feature 40: note = 3' end a SPAAC linker /3DBCO/
  source 1..40: mol_type = other RNA
tttttttttttttttttttttaaaaaaggaggagcaggga

SEQ ID NO: 4 - RNA, 21 nt, synthetic construct
  misc_feature 1..21: note = Synthetic
  misc_feature 1: note = 5' biotin tag
  misc_feature 21: note = seven (7) /iSp18/ hexapegs and thereafter /3ThioC3-D/ thiol modification at the 3' end
  source 1..21: mol_type = other RNA
ttttttttttttttttttttt

SEQ ID NO: 5 - RNA, 14 nt, synthetic construct
  misc_feature 1..14: note = Synthetic
  misc_feature 1: note = 5' biotin tag followed by two (2) /iSp18/ spacers
  misc_feature 14: note = 3' /3ThioC3-D/ thiol modification
  source 1..14: mol_type = other RNA
tttttttttttttt

SEQ ID NO: 6 - RNA, 10 nt, synthetic construct
  misc_feature 1..10: note = Synthetic
  misc_feature 1: note = 5' biotin tag followed by four (4) /iSp18/ hexapegs
  misc_feature 10: note = 3' /3ThioC3-D/ thiol modification
  source 1..10: mol_type = other RNA
tttttttttt

SEQ ID NO: 7 - RNA, 14 nt, synthetic construct
  misc_feature 1..14: note = Synthetic
  misc_feature 1: note = 5' biotin tag followed by two /iSp18/ hexapegs
  misc_feature 14: note = 3' /3DBCON/
  source 1..14: mol_type = other RNA
tttttttttttttt

SEQ ID NO: 8 - RNA, 10 nt, synthetic construct
  misc_feature 1..10: note = Synthetic
  misc_feature 10: note = 3' /3DBCON/
  source 1..10: mol_type = other RNA
  misc_feature 1: note = 5' biotin tag followed by three (3) /iSp18/ hexapegs
tttttttttt

SEQ ID NO: 9 - RNA, 14 nt, synthetic construct
  misc_feature 1..14: note = Synthetic
  misc_feature 1: note = 5' biotin tag followed by four (4) /iSp18/ hexapegs
  misc_feature 14: note = 3' /3DBCON/
  source 1..14: mol_type = other RNA
tttttttttttttt

SEQ ID NO: 10 - RNA, 21 nt, synthetic construct
  misc_feature 1..21: note = Synthetic
  misc_feature 1: note = 5' biotin tag followed by four (4) /iSp18/ hexapegs
  misc_feature 14: note = 3' /3DBCON/
  source 1..21: mol_type = other RNA
ttttttttttttttttttttt

SEQ ID NO: 11 - RNA, 21 nt, synthetic construct
  misc_feature 1..21: note = Synthetic
  misc_feature 1: note = 5/ biotin tag followed by four (4) /iSp18/ hexapegs
  misc_feature 21: note = 3' /3ThioC3-D/ thiol modification
  source 1..21: mol_type = other RNA
ttttttttttttttttttttt

SEQ ID NO: 12 - DNA, 308 bp, synthetic construct
  misc_feature 1..308: note = Synthetic
  source 1..308: mol_type = other DNA
gaaattaatacgactcactataggtgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttc

SEQ ID NO: 13 - DNA, 57 bp, synthetic construct
  misc_feature 1..57: note = Synthetic
  source 1..57: mol_type = other DNA
gaaattaatacgactcactatagggcaactcaggacttgttcttacctttcttttcc

SEQ ID NO: 14 - DNA, 26 bp, synthetic construct
  misc_feature 1..26: note = Synthetic
  source 1..26: mol_type = other DNA
aagcaaaataaacaccatcattaaat

SEQ ID NO: 15 - RNA, 63 nt, synthetic construct
  misc_feature 1..63: note = Synthetic
  source 1..63: mol_type = other RNA
gtttagactaccccaaaaacgaaggggactaaaacacagggttatcaaacctcttagtaccat

SEQ ID NO: 16 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacctaaaactattcacttcaatagtctgaa

SEQ ID NO: 17 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacaatatgattaccattaccacaaaaatta

SEQ ID NO: 18 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacttaatagtttcagccgcaaagagtctaa

SEQ ID NO: 19 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaactgttgtaacgcccttataatagacctta

SEQ ID NO: 20 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacgtcgcattttcccctcaatagagtcctt

SEQ ID NO: 21 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacaaaaagcttgtgaattcaaatgtccctg

SEQ ID NO: 22 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacactaaacagatcaggacaaggtatttgg

SEQ ID NO: 23 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacgtcgcaaaagacatggtctcctcttgtt

SEQ ID NO: 24 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacgtctttttctaggacattgtattgaaca

SEQ ID NO: 25 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaactccgagtcagtggctcccgtgtgtcggt

SEQ ID NO: 26 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacacgtctaatacgatacatcattacatat

SEQ ID NO: 27 - RNA, 64 nt, synthetic construct
  misc_feature 1..64: note = Synthetic
  misc_feature 64: note = 3' biotin tag
  source 1..64: mol_type = other RNA
gatttagactaccccaaaaacgaaggggactaaaacgtgcgccgttggctcgtgtagcagttcc

SEQ ID NO: 28 - DNA, 48 bp, synthetic construct
  misc_feature 1..48: note = Synthetic
  source 1..48: mol_type = other DNA
gaaattaatacgactcactatagggcaattagagatggaacttacacc

SEQ ID NO: 29 - DNA, 49 bp, synthetic construct
  misc_feature 1..49: note = Synthetic
  source 1..49: mol_type = other DNA
gaaattaatacgactcactataggggtgtgttaaaagtcaatctcctcg

SEQ ID NO: 30 - DNA, 51 bp, synthetic construct
  misc_feature 1..51: note = Synthetic
  source 1..51: mol_type = other DNA
gaaattaatacgactcactatagggacttgctaatgatgttaaagatacac

SEQ ID NO: 31 - DNA, 49 bp, synthetic construct
  misc_feature 1..49: note = Synthetic
  source 1..49: mol_type = other DNA
gaaattaatacgactcactataggggctaagaatgagagtagttcattg

SEQ ID NO: 32 - DNA, 51 bp, synthetic construct
  misc_feature 1..51: note = Synthetic
  source 1..51: mol_type = other DNA
gaaattaatacgactcactatagggtgatctcaatgaaattagaaagatgg

SEQ ID NO: 33 - DNA, 53 bp, synthetic construct
  misc_feature 1..53: note = Synthetic
  source 1..53: mol_type = other DNA
gaaattaatacgactcactataggggagcaaaaagaagtcctatataaataag

SEQ ID NO: 34 - DNA, 50 bp, synthetic construct
  misc_feature 1..50: note = Synthetic
  source 1..50: mol_type = other DNA
gaaattaatacgactcactatagggcaagcaaaacaaaaagactaaaggc

SEQ ID NO: 35 - DNA, 46 bp, synthetic construct
  misc_feature 1..46: note = Synthetic
  source 1..46: mol_type = other DNA
gaaattaatacgactcactatagggacccaaatgagaaagactgtg

SEQ ID NO: 36 - DNA, 45 bp, synthetic construct
  misc_feature 1..45: note = Synthetic
  source 1..45: mol_type = other DNA
gaaattaatacgactcactatagggcttcacgaaggctccacata

SEQ ID NO: 37 - DNA, 42 bp, synthetic construct
  misc_feature 1..42: note = Synthetic
  source 1..42: mol_type = other DNA
gaaattaatacgactcactatagggttgatgagctggagcca

SEQ ID NO: 38 - DNA, 24 bp, synthetic construct
  misc_feature 1..24: note = Synthetic
  source 1..24: mol_type = other DNA
ctttttagcttcttccacaatgtc

SEQ ID NO: 39 - DNA, 22 bp, synthetic construct
  misc_feature 1..22: note = Synthetic
  source 1..22: mol_type = other DNA
aaccataaggagcattttgaac

SEQ ID NO: 40 - DNA, 24 bp, synthetic construct
  misc_feature 1..24: note = Synthetic
  source 1..24: mol_type = other DNA
gacttaacactctcttctttagct

SEQ ID NO: 41 - DNA, 24 bp, synthetic construct
  misc_feature 1..24: note = Synthetic
  source 1..24: mol_type = other DNA
atttacagcactagaactttcatg

SEQ ID NO: 42 - DNA, 27 bp, synthetic construct
  misc_feature 1..27: note = Synthetic
  source 1..27: mol_type = other DNA
ctgatatctcgcttggaacatctgcag

SEQ ID NO: 43 - DNA, 22 bp, synthetic construct
  misc_feature 1..22: note = Synthetic
  source 1..22: mol_type = other DNA
aattagccacaaatccatagcg

SEQ ID NO: 44 - DNA, 30 bp, synthetic construct
  misc_feature 1..30: note = Synthetic
  source 1..30: mol_type = other DNA
tgtttcttcattatatctttctaatggtat

SEQ ID NO: 45 - DNA, 21 bp, synthetic construct
  misc_feature 1..21: note = Synthetic
  source 1..21: mol_type = other DNA
gcaacattaattcctgctgct

SEQ ID NO: 46 - DNA, 22 bp, synthetic construct
  misc_feature 1..22: note = Synthetic
  source 1..22: mol_type = other DNA
cccatattgttagtgatgcagg

SEQ ID NO: 47 - DNA, 20 bp, synthetic construct
  misc_feature 1..20: note = Synthetic
  source 1..20: mol_type = other DNA
atgtggatggctgagttgtt

SEQ ID NO: 48 - DNA, 18 bp, synthetic construct
  misc_feature 1..18: note = Synthetic
  source 1..18: mol_type = other DNA
gcacccatattgttagtg

SEQ ID NO: 49 - DNA, 525 bp, synthetic construct
  misc_feature 1..525: note = Synthetic
  source 1..525: mol_type = other DNA
gaaattaatacgactcactatagggatgctcttctttctattcagaatggttttagtgctaccaactctgcacttgctaaaatacaaagtgttgttaattctaatgctcaagcacttaatagtttgttacagcaattatttaataaatttggtgcaattagttcttctttacaagaaattttatctcgtctcgatgctttagaggctcaggttcagattgataggcttattaatggtcgtttaactgctttaaatgcttatgtctctcaacagcttagtgatatttctcttgtaaaatttggtgctgctttagctatggagaaggttaatgagtgtgttaaaagtcaatctcctcgtattaatttttgtggtaatggtaatcatattttgtcattagttcaaaatgctccttatggtttgttgtttatgcattttagttataaacctatttcttttaaaactgttttagtaagtcctggtttgtgtatatcaggtgatgtaggtattgcacctaaacaagggtat

SEQ ID NO: 50 - DNA, 705 bp, synthetic construct
  misc_feature 1..705: note = Synthetic
  source 1..705: mol_type = other DNA
gaaattaatacgactcactatagggacgttatgtgtctttagctattgatgcataccctctttcaaaacaccctaattctgaatatcgtaaggttttttacgtattacttgattgggttaagcatcttaacaaaaatttgaatgagggtgttcttgaatctttttctgttacacttcttgataatcaagaagataagttttggtgtgaagatttttatgctagtatgtatgaaaattctacaatattgcaagctgctggtttatgtgttgtttgtggttcacaaactgtacttcgttgtggtgattgtctgcgtaagcctatgttgtgcactaaatgcgcatatgatcatgtatttggtaccgaccacaagtttattttggctataacaccgtatgtatgtaatgcatcaggttgtggtgttagtgatgtcaaaaaattgtatcttggtggtttgaattactattgtacaaatcataaaccacagttgtcttttccattatgttcagctggtaatatatttggtttatataaaaattcagcaactggttccttagatgttgaagtttttaataggcttgcaacgtctgattggactgatgttagggactataaacttgctaatgatgttaaagatacacttagactctttgcggctgaaactattaaagctaaagaagagagtgttaagtcttcttatgcttttg

SEQ ID NO: 51 - DNA, 525 bp, synthetic construct
  misc_feature 1..525: note = Synthetic
  source 1..525: mol_type = other DNA
gaaattaatacgactcactataggggttgtagatgaagttagcatgcttaccaattatgagctttctgttattaatgctcgtattcgtgctaagcattatgtttatattggtgatcctgctcaattgccagcaccacgtgtgttattgagcaagggtacacttgaacctaaatattttaacactgttactaagctcatgtgttgcttagggccagacatttttcttggtacatgttatagatgtcctaaggaaattgttgatacagtgtccgccttggtttatgaaaataagcttaaggctaagaatgagagtagttcattgtgttttaaggtctattataagggcgttacaacacatgaaagttctagtgctgtaaatatgcagcagatttatttgattaataagtttttgaaggctaaccctttgtggcataaagctgtttttattagcccatataatagtcagaactttgcagctaagcgtgttttgggtttacaaacccaaaccgtggattctgctcaagg

SEQ ID NO: 52 - DNA, 525 bp, synthetic construct
  misc_feature 1..525: note = Synthetic
  source 1..525: mol_type = other DNA
gaaattaatacgactcactatagggaccatctgtcaaccagaaatcaaaccaacagaaacaagtgaaaaagatagtggatcaactgacaaaaatagacagtctgggtcatcacacgaatgtacaacagaagcaaaagatagaaatattgatcaggaaactgtacagagaggacctgggagaagaggcagctcagatagtagagctgagactgtggtctctggaggaatctccagaagcatcacagattctaaaaatggaacccaaaacacggagaatattgatctcaatgaaattagaaagatggataaggactctattgaggggaaaatgcgacaatctgcagatgttccaagcgagatatcaggaagtgatggcatatttacaacagaacaaagtagaaacagtgatcatggaagaagcttggaatctatcggtacacctgatacaagatcaataagtgttgttactgctgcaacaccagatgatgaagaagaaatactaatgagaaatagtaggatgaagaa

SEQ ID NO: 53 - DNA, 525 bp, synthetic construct
  misc_feature 1..525: note = Synthetic
  source 1..525: mol_type = other DNA
gaaattaatacgactcactatagggtgaatcaacaaggaagaaaattgagaagataaggcctcttttaatggatggcacagcatcactgagtcctgggatgatgatgggcatgttcaacatgctaagtacagtcttgggagtctcgatactgaatcttggacaaaagaaatacaccaagacaacatactggtgggatgggctccaatcatccgacgattttgctctcatagtgaatgcaccaaaccatgaaggaatacaagcaggagtggacagattctacaggacctgcaaattagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggctcagatggctcttc

SEQ ID NO: 54 - DNA, 525 bp, synthetic construct
  misc_feature 1..525: note = Synthetic
  source 1..525: mol_type = other DNA
gaaattaatacgactcactatagggcaagcaaaacaaaaagactaaaggcccaaataccttgtcctgatctgtttagtataccattagaaagatataatgaagaaacaagggcaaaattgaagaagctaaaaccattcttcaatgaagaaggaactgcatctttgtcacctgggatgatgatgggaatgtttaatatgctatctaccgtgttgggagtagctgcactaggtatcaagaacattggaaacaaagaatacctatgggatggactgcaatcttctgatgattttgctctatttgttaatgcaaaggatgaagaaacatgtatggaaggaataaacgacttttaccgaacatgtaaattattgggaataaacatgagcaaaaagaaaagttactgtaatgagactggaatgtttgaatttacaagcatgttctacagagatggatttgtatctaattttgcaatggaactcccttcgtttggggttgctggagtaaatgaatcagcagatatggcaata

SEQ ID NO: 55 - DNA, 226 bp, synthetic construct
  misc_feature 1..226: note = Synthetic
  source 1..226: mol_type = other DNA
gaaattaatacgactcactataggggagaagaccaagggtggtattgtcagaatgcagggtcaactgtttactacccaaatgagaaagactgtgaaacaagaggagaccatgtcttttgcgacacagcagcaggaattaatgttgctgagcaatcaaaggagtgcaacatcaacatatccactacaaattacccatgcaaagtcagcacaggaagacatcctatca

SEQ ID NO: 56 - DNA, 226 bp, synthetic construct
  misc_feature 1..226: note = Synthetic
  source 1..226: mol_type = other DNA
gaaattaatacgactcactatagggggggcaaatatggaaacatacgtgaacaaacttcacgaaggctccacatacacagctgctgttcaatacaatgtcctagaaaaagacgatgatcctgcatcacttacaatatgggtgcccatgttccaatcatccatgccagcagatttacttataaaagaactagctaatgtcaacatactagtgaaacaaatatccaca

SEQ ID NO: 57 - DNA, 340 bp, synthetic construct
  misc_feature 1..340: note = Synthetic
  source 1..340: mol_type = other DNA
gaaattaatacgactcactatagggtccttgcaggtggctgccaatacctccaccgtggagcttgttgatgagctggagccagagaccgacacacgggagccactgactcggatccgcaacaactcagccatccacatccgagtcttcagggtcacacccaagtaattgaaaagacactcctccacttatcccctccgtgatatggctcttcgcatgctgagtactggacctcggaccagagccatgtaagaaaaggcctgttccctggaagcccaaaggactctgcattgagggtgggggtaattgtctcttggtggcccagttagtgggccttcctga

SEQ ID NO: 58 - protein, 19 aa, synthetic construct
  REGION 1..19: note = Synthetic
  REGION 1..8: note = misc_feature - glycine-serine linker
  REGION 9..19: note = misc_feature - Either an N-terminal azido-lysine modification (for SPAAC) or with an N-terminal maleimide modification (for maleimide-thiol reactions)
GSSGGSSGVSGWRLFKKIS

SEQ ID NO: 59 - RNA, 73 nt, synthetic construct
  misc_feature 1..73: note = Synthetic
  source 1..73: mol_type = other RNA
cacgaagcaaccacaggttttttttttttttttttttttttttttttttttttttcaccagcgaaaggaagag

SEQ ID NO: 60 - RNA, 37 nt, synthetic construct
  misc_feature 1..37: note = Synthetic
  misc_feature 37: note = 3' /3DBCO/ or 3' thiol modification
  source 1..37: mol_type = other RNA
tttttttttttttttttttcaccagcgaaaggaagag

SEQ ID NO: 61 - DNA, 22 bp, synthetic construct
  misc_feature 1..22: note = Synthetic
  misc_feature 1: note = 5' /HaloLigand/
  source 1..22: mol_type = other DNA
ctcttcactattacgctaggtg